(19) United States
(12) Patent Application Publication
(10) Pub. No.: US 2025/0082624 A1
(43) Pub. Date: Mar. 13, 2025

(54) METHODS OF TREATING COGNITIVE IMPAIRMENT

(71) Applicant: NeuroTherapia, Inc., Cleveland, OH (US)

(72) Inventors: Tony Giordano, Chesterland, OH (US); Joseph Foss, Gates Mills, OH (US)

(21) Appl. No.: 18/827,231

(22) Filed: Sep. 6, 2024

Related U.S. Application Data
(60) Provisional application No. 63/545,041, filed on Oct. 20, 2023.
(60) Provisional application No. 63/537,045, filed on Sep. 7, 2023.

(51) Int. Cl. A61K 31/4525

(52) U.S. Cl. CPC A61K 31/4525

(57) ABSTRACT

The present disclosure is directed to use of the compound of formula I to treat neurodegeneration in human subjects.

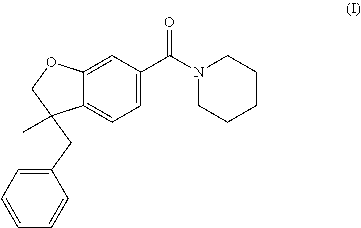 [SMILES: CC1(Cc2ccccc2)COc2cc(C(=O)N3CCCCC3)ccc21]

### CROSS-REFERENCE TO RELATED APPLICATIONS

This application claims the benefit of U.S. Provisional Application No. 63/537,045, filed Sep. 7, 2023, and U.S. Provisional Application No. 63/545,041, filed Oct. 20, 2023, each of which is incorporated by reference herein in its entirety.

### BACKGROUND

CB1 and CB2 are two cannabinoid receptors that belong to the GPCR family and have very different functions and distribution. While no x-ray structure is available for these receptors, various models have been described on the basis of the x-ray structure of rhodopsin, a GPCR belonging protein responsible of the light sensitivity in vision. Matsuda L A, Lolait S J, Brownstein M J, Young A C, Bonner T I, “Structure of a Cannabinoid Receptor and Functional Expression of the Cloned cDNA,” Nature 1990, 346:561-4. CB1 is abundantly expressed in the central nervous system and is most dense in the basal ganglia, cerebellum, hippocampus, and cortex and in the peripheral nervous system, it is expressed in such sites as the testis, eye, urinary bladder, and adipocytes. CB2 is mainly expressed in the immune tissues, in cells such as those in the thymus, marrow, spleen, pancreas, and in glioma and skin tumor cells. It was recently demonstrated that CB2 receptors and their gene transcripts are widely distributed in the brain. A third cannabinoid receptor seems to be present as some chemical analogues exhibit cannabinoid biological activity without activating CB1 and CB2. Di Marzo V, Bifulco M, De Petrocellis L, “The Endocannabinoid System and Its Therapeutic Exploitation,” Nat. Rev. Drug Discov. 2004, 3:771-84.

With the exception of a small population of neurons located in the brain stem and the cerebellum, healthy brain tissue does not express CB2 receptors. Van Sickle et al., Science 310, 329-332 (2005). Rather, CB2 receptors are upregulated in reactive microglial cells in Alzheimer's disease (AD), Huntington's disease, simian immunodeficiency virus-induced encephalitis, HIV encephalitis, and multiple sclerosis. In vitro studies demonstrated that CB2-selective agonists blocked microglia-mediated neurotoxicity after A13 is added to rat cortical co-cultures. Furthermore, intracerebroventricular administration of nonselective cannabinoid receptor agonist WIN 55,212-2 to rats prevented A13-induced microglial activation, cognitive impairment, and loss of neuronal markers. CB2 agonists are neuroprotective and they have the advantage of lacking the psychotropic adverse effects normally seen with CB1 agonists. Ramirez et al., J. Neurosci. 25, 1904-1913 (2005). In contrast, CB1 agonists appear to have no beneficial effects on AD neuropathology and behavioral deficits in a mouse model of AD Chen et al., Curr Alzheimer Res. 7, 255-261 (2010).

Studies show that in the settings of AD, microglia and astrocytes become fully reactive, initiating a proinflammatory cascade that results in the release of potentially neurotoxic substances, including cytokines, which lead to degenerative changes in neurons. CB2 is also upregulated during this process. Accordingly, safe and effective methods for treating AD and cognitive impairment using CB2 receptor agonists are needed.

### SUMMARY

In some aspects, the present disclosure provides methods of treating neurodegeneration, Alzheimer's disease and/or cognitive impairment in a human subject in need thereof (e.g., a subject with mild cognitive impairment and/or early Alzheimer's disease). In some such embodiments, the method comprises:
a) on day 1, administering to the subject a first daily dose that is a pharmaceutical composition comprising 80-150 mg of the compound of Formula I

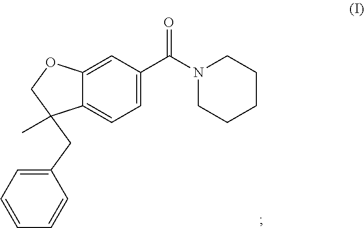 [SMILES: CC1(Cc2ccccc2)COc2cc(C(=O)N3CCCCC3)ccc21]

b) starting on day 2, administering to the subject a subsequent daily dose that is a pharmaceutical composition comprising 80-150 mg of the compound of Formula I; and
c) 6-28 days after the first daily dose (e.g., 14-28 days after the first daily dose), increasing the subsequent daily dose to a pharmaceutical composition comprising 125-225 mg of the compound of Formula I (e.g., increasing the subsequent daily dose to at least 150% of the first daily dose).

In other aspects, the present disclosure provides methods of treating neurodegeneration, Alzheimer's disease and/or cognitive impairment in a human subject in need thereof (e.g., a subject with mild cognitive impairment and/or early Alzheimer's disease). In some such embodiments, the method comprises:
a) on day 1, administering to the subject a first daily dose that is a pharmaceutical composition comprising 90-135 mg of the compound of Formula I;
b) starting on day 2, administering to the subject a subsequent daily dose that is a pharmaceutical composition comprising 90-135 mg of the compound of Formula I; and
c) 6-28 days after the first daily dose (e.g., 14-28 days after the first daily dose), increasing the subsequent daily dose to a pharmaceutical composition comprising 135-202.5 mg of the compound of Formula I (e.g., increasing the subsequent daily dose to at least 150% of the first daily dose).

In still other aspects, the present disclosure provides methods of treating neurodegeneration Alzheimer's disease and/or cognitive impairment in a human subject in need thereof (e.g., a subject with mild cognitive impairment and/or early Alzheimer's disease). In some such embodiments, the method comprises:
a) on day 1, administering to the subject a first daily dose that is a pharmaceutical composition comprising 80-150 mg of the compound of Formula I;
b) starting on day 2, administering to the subject a subsequent daily dose that is a pharmaceutical composition comprising 80-150 mg of the compound of Formula I;
c) 6-28 days after the first daily dose (e.g., 14-28 days after the first daily dose), measuring the serum level of the compound of Formula I in the subject at a time point in the period 1-12 hours after that day's daily dose;
d) plotting the serum level of the compound of Formula I measured in c) against the graph of serum levels in FIG. 1 at the time point corresponding to the time point in c); and
e) if the serum level is below the geometric mean in FIG. 1, increasing the subsequent daily dosage by an amount corresponding to the percentage increase required to reach the geometric mean.

In other aspects, the present disclosure provides methods of treating neurodegeneration, Alzheimer's disease and/or cognitive impairment in a human subject in need thereof (e.g., a subject with mild cognitive impairment and/or early Alzheimer's disease). In some such embodiments, the method comprises:
a) on day 1, administering to the subject a first daily dose that is a pharmaceutical composition comprising 90-180 mg of the compound of Formula I;
b) starting on day 2, administering to the subject a subsequent daily dose that is a pharmaceutical composition comprising 90-180 mg of the compound of Formula I; and
c) 6-28 days after the first daily dose (e.g., 14-28 days after the first daily dose), increasing the subsequent daily dose to a pharmaceutical composition comprising 135-270 mg of the compound of Formula I (e.g., increasing the subsequent daily dose to a pharmaceutical composition comprising 150-200% of the compound of Formula I in the subsequent daily dose administered in step b).

In still other aspects, the present disclosure provides methods of treating neurodegeneration, Alzheimer's disease and/or cognitive impairment in a human subject in need thereof (e.g., a subject with mild cognitive impairment and/or early Alzheimer's disease). In some such embodiments, the method comprises:
a) on day 1, administering to the subject a first daily dose that is a pharmaceutical composition comprising 90-180 mg of the compound of Formula I;
b) starting on day 2, administering to the subject a subsequent daily dose that is a pharmaceutical composition comprising 90-180 mg of the compound of Formula I; and
c) 6-28 days after the first daily dose (e.g., 14-28 days after the first daily dose), measuring the AUC of the compound of Formula I in the subject; and
d) if the AUC is less than 1200 h·ng/mL, increasing the subsequent daily dose by an amount corresponding to the percentage increase required to reach an AUC of at least 1200 h·ng/mL.

In still other aspects, the present disclosure provides methods of treating neurodegeneration, Alzheimer's disease and/or cognitive impairment in a human subject in need thereof (e.g., a subject with mild cognitive impairment and/or early Alzheimer's disease). In some such embodiments, the method comprises:
a) on day 1, administering to the subject a first daily dose that is a pharmaceutical composition comprising 90-180 mg of the compound of Formula I;
b) starting on day 2, administering to the subject a subsequent daily dose that is a pharmaceutical composition comprising 90-180 mg of the compound of Formula I;
c) 6-28 days after the first daily dose (e.g., 14-28 days after the first daily dose), increasing the subsequent daily dose to a pharmaceutical composition comprising 125-225% of the amount of the compound of Formula I in the subsequent daily dose administered in step b.

### BRIEF DESCRIPTION OF THE DRAWINGS

FIG. 1 provides pharmacokinetic data for subjects in Cohort D (90 mg dose) on Day 1 of once daily administration of a 90-mg dose of NTRX-07 for seven days. The solid line represents the geometric mean of the data.

FIG. 2 provides a schematic overview of certain aspects of microglial-mediated neuroinflammation.

FIG. 3 shows that NTRX-07 (formerly known as MDA7) mitigates microglial activation via the CB2 receptor. The study used an APP/PS1 mouse model of AD and observed colocalization of Iba1 in microglia and CB2 receptors. CB2 was very low in wild-type and markedly increased in AD. The increase returned towards baseline with NTRX-07 treatment.

FIG. 4 shows that NTRX-07 increases clearance of Aβ.

FIG. 5 shows that NTRX-07 promotes Aβ plaque clearance in a APP/PS1 (Tg(APPswe,PSEN1dE9)85Dbo) mouse model of AD.

FIG. 6 shows that NTRX-07 restores memory in preclinical models. In the study shown, NTRX-07-treated animals showed no difference from control animals.

FIG. 7 provides an overview of the study described herein in Example 2.

FIG. 8A shows the plasma levels of NTRX-07 cohorts on Day 1 in the study described herein in Example 2. FIG. 8B shows the plasma levels of NTRX-07 cohorts on Day 7 in the study described herein in Example 2.

FIG. 9 shows the change in EEG power observed with NTRX-07 treatment in the study described herein in Example 2, with overall mean frequency (MF) shown as time match ratios to account for potential Circadian effects.

FIG. 10 shows an example of the expected decrease in mean frequency in AD patients referenced herein in Example 3. FIG. 10 is adapted from FIG. 1 of Clinical Neurophysiology (2007) 118:186-196 and shows the log-transformed mean relative power spectra across the 19 channels of the two subject groups. The illustrated areas cover ±1 SE around the mean values; black area, AD patients; gray area, controls.

FIG. 11 shows the observation of the expected drop in mean frequency described herein in Example 3.

FIGS. 12A and 12B show the plasma NTRX-07 PK data from administration of a 10 mg NTRX-07 dose to healthy volunteers on Day 1 (FIG. 12A) and Day 7 (FIG. 12B) as described herein in Example 3.

FIGS. 12C and 12D show the plasma NTRX-07 PK data from administration of a 30 mg NTRX-07 dose to healthy volunteers on Day 1 (FIG. 12C) and Day 7 (FIG. 12D) as described herein in Example 3.

FIGS. 12E and 12F show the plasma NTRX-07 PK data from administration of a 90 mg NTRX-07 dose to healthy volunteers on Day 1 (FIG. 12E) and Day 7 (FIG. 12F) as described herein in Example 3.

FIGS. 12G and 12H show the plasma NTRX-07 PK data from administration of a 90 mg NTRX-07 dose to AD subjects on Day 1 (FIG. 12G) and Day 7 (FIG. 12H) as described herein in Example 3.

FIGS. 13A-13P show the change from baseline in biomarkers after 7 days of 90 mg NTRX-07 treatment in the AD cohort as described herein in Example 3: IL-12p70 (FIG. 13A), IL-10 (FIG. 13B), IL-4 (FIG. 13C), IL-5 (FIG. 13D), INFg (FIG. 13E), IL-6 (FIG. 13F), IL-8 (FIG. 13G), IL-22 (FIG. 13H), TNFa (FIG. 13I), IL-10 (FIG. 13J), hCRP (FIG. 13K), Chitinase-3 (FIG. 13L), TREM-2 (FIG. 13M), IL-2 (FIG. 13N), Neurogranin (FIG. 13O), AB42 (FIG. 13P).

FIGS. 14A-14P show the change from baseline to day 14 in biomarkers after 7 days of 90 mg NTRX-07 treatment in the AD cohort as described herein in Example 3: NF-LIGHT (FIG. 14A), IL-12p70 (FIG. 14B), IL-10 (FIG. 14C), IL-4 (FIG. 14D), IL-5 (FIG. 14E), INFg (FIG. 14F), IL-6 (FIG. 14G), IL-8 (FIG. 14H), IL-22 (FIG. 14I), TNFa (FIG. 14J), IL-10 (FIG. 14K), hCRP (FIG. 14L), Chitinase-3 (FIG. 14M), TREM-2 (FIG. 14N), Neurogranin (FIG. 14O), NF-LIGHT (FIG. 14P).

FIGS. 15A and 15B show NTRX-07 PK data from QD administration of a 10 mg dose (MAD Cohort A) on Day 1 (FIG. 15A) and Day 7 (FIG. 15B) as described in Example 4.

FIGS. 16A and 16B show NTRX-07 PK data from QD administration of a 30 mg dose (MAD Cohort B) on Day 1 (FIG. 16A) and Day 7 (FIG. 16B) as described in Example 4.

FIG. 17 shows NTRX-07 PK data from administration of a 30 mg dose in the fed state (MAD Cohort B Fed) as described in Example 4.

FIGS. 18A and 18B show NTRX-07 PK data from QD administration of a 90 mg dose (MAD Cohort C) highlighting an outlier subject on Day 1 (FIG. 18A) and Day 7 (FIG. 18B) as described in Example 4.

FIGS. 19A and 19B show NTRX-07 PK data from QD administration of a 90 mg dose (MAD Cohort D) on Day 1 (FIG. 19A) and Day 7 (FIG. 19B) as described in Example 4.

FIGS. 20A and 20B show geometric mean PK data for NTRX-07 cohorts on Day 1 (FIG. 20A) and Day 7 (FIG. 20B) as described in Example 4.

FIG. 21 shows a PK scheme for absorption, distribution, and clearance of NTRX-07 as described in Examples 4-8.

FIGS. 22A and 22B show a Compartmental PK model for NTRX-07 compared to Day 1 (FIG. 22A) and Day 7 (FIG. 22B) mean data as described in Example 4.

FIGS. 23A and 23B show NTRX-07 PK data from QD administration of a 40 mg dose (MAD Cohort A) on Day 1 (FIG. 23A) and Day 7 (FIG. 23B) as described in Example 5.

FIGS. 24A and 24B show a Compartmental PK model for NTRX-07 compared to Day 1 (FIG. 24A) and Day 7 (FIG. 24B) data as described in Example 5.

FIGS. 25A and 25B show NTRX-07 PK data from QD administration of a 120 mg dose (MAD Cohort B) on Day 1 (FIG. 25A) and Day 7 (FIG. 25B) as described in Example 6.

FIGS. 26A and 26B show a Compartmental PK model for NTRX-07 compared to Day 1 (FIG. 26A) and Day 7 (FIG. 26B) data as described in Example 6.

FIGS. 27A and 27B show a Comparison of PK outputs for different doses showing 120-mg PK normalized to 40 mg on Day 1 (FIG. 27A) and Day 7 (FIG. 27B) as described in Example 6.

FIGS. 28A and 28B show NTRX-07 PK data from QD administration of a 360 mg dose (MAD Cohort C) on Day 1 (FIG. 28A) and Day 7 (FIG. 28B) as described in Example 7.

FIGS. 29A and 29B show Compartmental PK model for NTRX-07 compared to Day 1 (FIG. 29A) and Day 7 (FIG. 29B) data as described in Example 7.

FIGS. 30A and 30B show a Comparison of PK outputs for different doses normalized to a 40-mg dose on Day 1 (FIG. 30A) and Day 7 (FIG. 30B) as described in Example 7.

FIGS. 31A and 31B show NTRX-07 PK data from QD administration of a 360 mg SDD dose (MAD Cohort D) on Day 1 (FIG. 31A) and Day 7 (FIG. 31B) as described in Example 8.

FIGS. 32A and 32B show a Compartmental PK model for NTRX-07 compared to Day 1 (FIG. 32A) and Day 7 (FIG. 32B) data as described in Example 8.

### DETAILED DESCRIPTION

The present disclosure is directed to methods of treating neurodegeneration using the compound of Formula I:

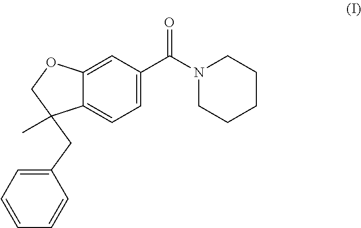 [SMILES: CC1(Cc2ccccc2)COc2cc(C(=O)N3CCCCC3)ccc21]

Synthesis and characterization of the compound of Formula I is described in U.S. Pat. No. 8,440,832, which is incorporated herein by reference in its entirety. Spray-dried dispersions (SDDs) of the compound of Formula I, and preparation and characterization thereof, are described in U.S. Pat. No. 10,526,318, which is incorporated herein by reference in its entirety.

In some aspects, the present disclosure provides methods of treating neurodegeneration, Alzheimer's disease and/or cognitive impairment in a human subject in need thereof (e.g., a subject with mild cognitive impairment and/or early Alzheimer's disease). In some such embodiments, the method comprises:
a) on day 1, administering to the subject a first daily dose that is a pharmaceutical composition comprising 80-150 mg of the compound of Formula I;
b) starting on day 2, administering to the subject a subsequent daily dose that is a pharmaceutical composition comprising 80-150 mg of the compound of Formula I;
c) 6-28 days after the first daily dose, increasing the subsequent daily dose to a pharmaceutical composition comprising 125-225 mg of the compound of Formula I. As used herein, subsequent daily dosing “starting on day 2” repeats daily on day 3, day 4, day 5, day 6, day 7, day 8, and so on. As used herein, “6-28 days after the first daily dose” is day 7 to day 29, i.e., the subsequent daily dosing is increased starting on a day that is day 7, day 8, day 9, day 10, day 11, day 12, day 13, day 14, day 15, day 16, day 17, day 18, day 19, day 20, day 21, day 22, day 23, day 24, day 25, day 26, day 27, day 28, or day 29, to 125-225 mg of the compound of Formula I. In certain embodiments, step c is performed 6 days after the first daily dose.

In certain embodiments, pharmaceutical compositions comprising the compound of Formula I that may be suitable for use in the methods of the present disclosure include, but are not limited to, SDDs described in U.S. Pat. No. 10,526,318, which is incorporated herein by reference in its entirety. As described in U.S. Pat. No. 10,526,318, SDDs are produced by dissolving a compound of the invention (e.g., the compound of Formula I) and a pharmaceutically acceptable carrier (which may be a polymer, such as a cellulose derivative, such as hydroxypropyl methylcellulose acetate succinate (HPMCAS)) in an organic solvent or co-solvent mixture, then atomizing the solution into fine droplets in a drying chamber. The drying medium—typically heated nitrogen gas—evaporates the solvent, leaving the dry amorphous solid dispersion to be collected. Due to rapid solvent evaporation, SDDs achieve a thorough mixing of the compound of the invention and the carrier (e.g., the polymer carrier). The SDDs are also flowable and compressible, allowing them to be compressed, for example, into tablets for oral administration. HPMCAS have been demonstrated to be particularly effective in forming amorphous solid dispersions with poorly soluble active pharmaceutical ingredients such as cannabinoid receptor modulators that result in solubility enhancement through the ability to achieve and sustain a supersaturated solution of the active pharmaceutical ingredient. Curatolo et al., Pharmaceutical Research, 26(6), pp. 1419-1431 (2009). The extent of solubility enhancement and the sustainment is dependent on the acetate and succinate content of the polymer and varies depending on the specific active pharmaceutical ingredient being administered.

In some embodiments, the first daily dose is a pharmaceutical composition comprising 80-150 mg of the compound of Formula I, such as 96-150 mg, 104-150 mg, 120-150 mg, 80-130 mg, 96-130 mg, 104-130 mg, 120-130 mg, 80-120 mg, 96-120 mg, 104-120 mg, 80-100 mg, or 96-100 mg of the compound of Formula I. Accordingly, in various embodiments, the pharmaceutical composition may comprise 80, 81, 82, 83, 84, 85, 86, 87, 88, 89, 90, 91, 92, 93, 94, 95, 96, 97, 98, 99, 100, 101, 102, 103, 104, 105, 106, 107, 108, 109,110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 mg of the compound of Formula I, or any amount therebetween.

In some embodiments, the subsequent daily dose administered to the subject starting on day 2 is a pharmaceutical composition comprising 80-150 mg of the compound of Formula I, such as 96-150 mg, 104-150 mg, 120-150 mg, 80-130 mg, 96-130 mg, 104-130 mg, 120-130 mg, 80-120 mg, 96-120 mg, 104-120 mg, 80-100 mg, or 96-100 mg of the compound of Formula I. Accordingly, in various embodiments, the pharmaceutical composition may comprise 80, 81, 82, 83, 84, 85, 86, 87, 88, 89, 90, 91, 92, 93, 94, 95, 96, 97, 98, 99, 100, 101, 102, 103, 104, 105, 106, 107, 108, 109,110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 mg of the compound of Formula I, or any amount therebetween.

In some embodiments, 6-28 days after the first daily dose (i.e., in step c), the subsequent daily dose is increased to a pharmaceutical composition comprising 125-225 mg of the compound of Formula I, such as 150-225 mg, 162.5-225 mg, 187.5-225 mg, 125-195 mg, 150-195 mg, 162.5-195 mg, 187.5-195 mg, 125-180 mg, 150-180 mg, 162.5-180 mg, or 125-150 mg of the compound of Formula I. Accordingly, in various embodiments, the pharmaceutical composition may comprise 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, or 225 mg of the compound of Formula I, or any amount therebetween.

In some embodiments, the neurodegeneration comprises amyotrophic lateral sclerosis (ALS), Huntington's Disease, Parkinson's Disease, Alzheimer's Disease, cognitive impairment, or a combination thereof, or other disorders involving microglia activation and/or neuroflammation. In certain such embodiments, the neurodegeneration comprises Alzheimer's Disease, cognitive impairment, or a combination thereof. The Alzheimer's Disease may be mild or moderate, and it maybe early or late. The cognitive impairment may be mild or moderate. In some embodiments, the Alzheimer's Disease is prodromal. In some embodiments, the neurodegeneration comprises Alzheimer's Disease with cognitive impairment, such as early Alzheimer's Disease with mild cognitive impairment. As used herein, prodromal Alzheimer's Disease is characterized by mild cognitive impairment due to Alzheimer's Disease plus very mild Alzheimer's Disease dementia, as described in Practical Neurology (June 2019) 36-47, which is hereby incorporated by reference herein in its entirety. Methods and standards for diagnosing and determining the different types, levels, and/or stages of Alzheimer's Disease, and the presence, absence, and/or level of cognitive impairment, are known to those of skill in the art (e.g., clinicians, physicians) and include, but are not limited to, those described in Practical Neurology (June 2019) 36-47, which is hereby incorporated by reference herein in its entirety.

In other aspects, the present disclosure provides methods of treating neurodegeneration, Alzheimer's disease and/or cognitive impairment in a human subject in need thereof (e.g., a subject with mild cognitive impairment and/or early Alzheimer's disease). In some such embodiments, the method comprises:
a) on day 1, administering to the subject a first daily dose that is a pharmaceutical composition comprising 90-135 mg of the compound of Formula I;
b) starting on day 2, administering to the subject a subsequent daily dose that is a pharmaceutical composition comprising 90-135 mg of the compound of Formula I;
c) 6-28 days after the first daily dose, increasing the subsequent daily dose to a pharmaceutical composition comprising 135-202.5 mg of the compound of Formula I. As used herein, subsequent daily dosing “starting on day 2” repeats daily on day 3, day 4, day 5, day 6, day 7, day 8, and so on. As used herein, “6-28 days after the first daily dose” is day 7 to day 29, i.e., the subsequent daily dosing is increased starting on a day that is day 7, day 8, day 9, day 10, day 11, day 12, day 13, day 14, day 15, day 16, day 17, day 18, day 19, day 20, day 21, day 22, day 23, day 24, day 25, day 26, day 27, day 28, or day 29, to 135-202.5 mg of the compound of Formula I. In certain embodiments, step c is performed 6 days after the first daily dose.

In certain embodiments, pharmaceutical compositions comprising the compound of Formula I that may be suitable for use in the methods of the present disclosure include, but are not limited to, SDDs described in U.S. Pat. No. 10,526,318, which is incorporated herein by reference in its entirety. As described in U.S. Pat. No. 10,526,318, SDDs are produced by dissolving a compound of the invention (e.g., the compound of Formula I) and a pharmaceutically acceptable carrier (which may be a polymer, such as a cellulose derivative, such as hydroxypropyl methylcellulose acetate succinate (HPMCAS)) in an organic solvent or co-solvent mixture, then atomizing the solution into fine droplets in a drying chamber. The drying medium—typically heated nitrogen gas—evaporates the solvent, leaving the dry amorphous solid dispersion to be collected. Due to rapid solvent evaporation, SDDs achieve a thorough mixing of the compound of the invention and the carrier (e.g., the polymer carrier). The SDDs are also flowable and compressible, allowing them to be compressed, for example, into tablets for oral administration. HPMCAS have been demonstrated to be particularly effective in forming amorphous solid dispersions with poorly soluble active pharmaceutical ingredients such as cannabinoid receptor modulators that result in solubility enhancement through the ability to achieve and sustain a supersaturated solution of the active pharmaceutical ingredient. Curatolo et al., Pharmaceutical Research, 26(6), pp. 1419-1431 (2009). The extent of solubility enhancement and the sustainment is dependent on the acetate and succinate content of the polymer and varies depending on the specific active pharmaceutical ingredient being administered.

In some embodiments, the first daily dose is a pharmaceutical composition comprising 90-135 mg of the compound of Formula I, such as 108-135 mg, 117-135 mg, 90-117 mg, 108-117 mg, 90-108 mg, 90 mg, 108 mg, 117 mg, or 135 mg of the compound of Formula I. Accordingly, in various embodiments, the pharmaceutical composition may comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, or 135 mg of the compound of Formula I, or any amount therebetween.

In some embodiments, the subsequent daily dose administered to the subject starting on day 2 is a pharmaceutical composition comprising 90-135 mg of the compound of Formula I, such as 108-135 mg, 117-135 mg, 90-117 mg, 108-117 mg, 90-108 mg, 90 mg, 108 mg, 117 mg, or 135 mg of the compound of Formula I. Accordingly, in various embodiments, the pharmaceutical composition may comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, or 135 mg of the compound of Formula I, or any amount therebetween.

In some embodiments, 6-28 days after the first daily dose (i.e., in step c), the subsequent daily dose is increased to a pharmaceutical composition comprising 135-202.5 mg of the compound of Formula I, such as 162-202.5 mg, 175.5-202.5 mg, 135-175.5 mg, 162-175.5 mg, 135-162 mg, 135 mg, 162 mg, 175.5 mg, or 202.5 mg of the compound of Formula I. Accordingly, in various embodiments, the pharmaceutical composition may comprise 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, or 202.5 mg of the compound of Formula I, or any amount therebetween.

In some embodiments, the neurodegeneration comprises amyotrophic lateral sclerosis (ALS), Huntington's Disease, Parkinson's Disease, Alzheimer's Disease, cognitive impairment, or a combination thereof, or other disorders involving microglia activation and/or neuroflammation. In certain such embodiments, the neurodegeneration comprises Alzheimer's Disease, cognitive impairment, or a combination thereof. The Alzheimer's Disease may be mild or moderate, and it maybe early or late. The cognitive impairment may be mild or moderate. In some embodiments, the Alzheimer's Disease is prodromal. In some embodiments, the neurodegeneration comprises Alzheimer's Disease with cognitive impairment, such as early Alzheimer's Disease with mild cognitive impairment. As used herein, prodromal Alzheimer's Disease is characterized by mild cognitive impairment due to Alzheimer's Disease plus very mild Alzheimer's Disease dementia, as described in Practical Neurology (June 2019) 36-47, which is hereby incorporated by reference herein in its entirety. Methods and standards for diagnosing and determining the different types, levels, and/or stages of Alzheimer's Disease, and the presence, absence, and/or level of cognitive impairment, are known to those of skill in the art (e.g., clinicians, physicians) and include, but are not limited to, those described in Practical Neurology (June 2019) 36-47, which is hereby incorporated by reference herein in its entirety.

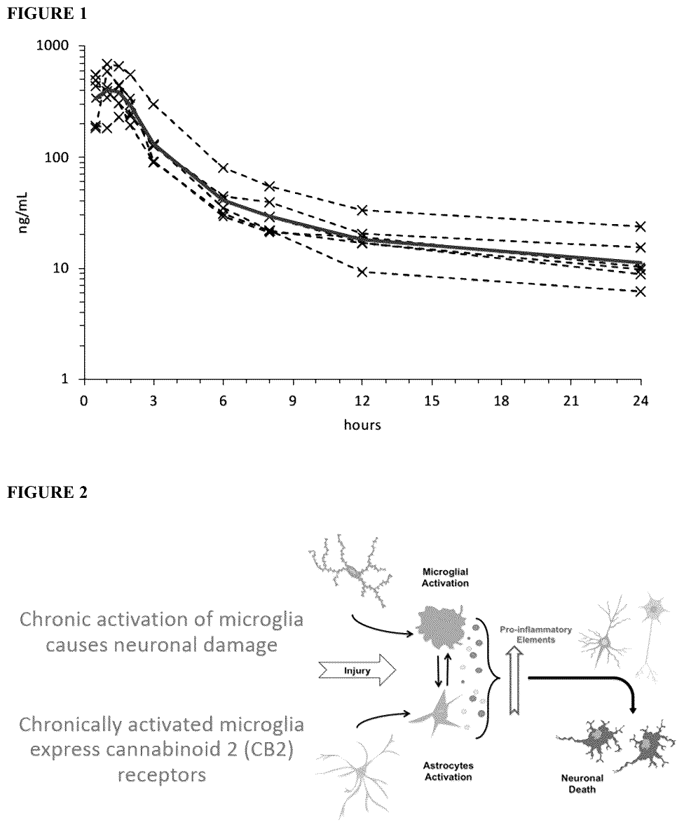

In still other aspects, the present disclosure provides methods of treating neurodegeneration, Alzheimer's disease and/or cognitive impairment in a human subject in need thereof (e.g., a subject with mild cognitive impairment and/or early Alzheimer's disease). In some such embodiments, the method comprises:
a) on day 1, administering to the subject a first daily dose that is a pharmaceutical composition comprising 80-150 mg of the compound of Formula I;
b) starting on day 2, administering to the subject a subsequent daily dose that is a pharmaceutical composition comprising 80-150 mg of the compound of Formula I;
c) 6-28 days after the first daily dose, measuring the serum level of the compound of Formula I in the subject at a time point in the period 1-12 hours after that day's daily dose;
d) plotting the serum level of the compound of Formula I measured in c) against the graph of serum levels in FIG. 1 at the time point corresponding to the time point in c);
e) if the serum level is below the geometric mean in FIG. 1, increasing the subsequent daily dosage by an amount corresponding to the percentage increase required to reach the geometric mean. As used herein, subsequent daily dosing “starting on day 2” repeats daily on day 3, day 4, day 5, day 6, day 7, day 8, and so on. As used herein, “6-28 days after the first daily dose” is day 7 to day 29, i.e., if the serum level is below the geometric mean in FIG. 1, the subsequent daily dosing is increased starting on a day that is day 7, day 8, day 9, day 10, day 11, day 12, day 13, day 14, day 15, day 16, day 17, day 18, day 19, day 20, day 21, day 22, day 23, day 24, day 25, day 26, day 27, day 28, or day 29, by an amount corresponding to the percentage increase required to reach the geometric mean. In certain embodiments, step c is performed 6 days after the first daily dose. As used herein, the geometric mean refers to the solid line (i.e., not the dashed lines) depicted in FIG. 1. In certain embodiments, pharmaceutical compositions comprising the compound of Formula I that may be suitable for use in the methods of the present disclosure include, but are not limited to, SDDs described in U.S. Pat. No. 10,526,318, which is incorporated herein by reference in its entirety. As described in U.S. Pat. No. 10,526,318, SDDs are produced by dissolving a compound of the invention (e.g., the compound of Formula I) and a pharmaceutically acceptable carrier (which may be a polymer, such as a cellulose derivative, such as hydroxypropyl methylcellulose acetate succinate (HPMCAS)) in an organic solvent or co-solvent mixture, then atomizing the solution into fine droplets in a drying chamber. The drying medium—typically heated nitrogen gas—evaporates the solvent, leaving the dry amorphous solid dispersion to be collected. Due to rapid solvent evaporation, SDDs achieve a thorough mixing of the compound of the invention and the carrier (e.g., the polymer carrier). The SDDs are also flowable and compressible, allowing them to be compressed, for example, into tablets for oral administration. HPMCAS have been demonstrated to be particularly effective in forming amorphous solid dispersions with poorly soluble active pharmaceutical ingredients such as cannabinoid receptor modulators that result in solubility enhancement through the ability to achieve and sustain a supersaturated solution of the active pharmaceutical ingredient. Curatolo et al., Pharmaceutical Research, 26(6), pp. 1419-1431 (2009). The extent of solubility enhancement and the sustainment is dependent on the acetate and succinate content of the polymer and varies depending on the specific active pharmaceutical ingredient being administered.

In some embodiments, the first daily dose is a pharmaceutical composition comprising 80-150 mg of the compound of Formula I, such as 96-150 mg, 104-150 mg, 120-150 mg, 80-130 mg, 96-130 mg, 104-130 mg, 120-130 mg, 80-120 mg, 96-120 mg, 104-120 mg, 80-100 mg, or 96-100 mg of the compound of Formula I. Accordingly, in various embodiments, the pharmaceutical composition may comprise 80, 81, 82, 83, 84, 85, 86, 87, 88, 89, 90, 91, 92, 93, 94, 95, 96, 97, 98, 99, 100, 101, 102, 103, 104, 105, 106, 107, 108, 109,110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 mg of the compound of Formula I, or any amount therebetween.

In some embodiments, the subsequent daily dose administered to the subject starting on day 2 is a pharmaceutical composition comprising 80-150 mg of the compound of Formula I, such as 96-150 mg, 104-150 mg, 120-150 mg, 80-130 mg, 96-130 mg, 104-130 mg, 120-130 mg, 80-120 mg, 96-120 mg, 104-120 mg, 80-100 mg, or 96-100 mg of the compound of Formula I. Accordingly, in various embodiments, the pharmaceutical composition may comprise 80, 81, 82, 83, 84, 85, 86, 87, 88, 89, 90, 91, 92, 93, 94, 95, 96, 97, 98, 99, 100, 101, 102, 103, 104, 105, 106, 107, 108, 109,110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 mg of the compound of Formula I, or any amount therebetween.

In some embodiments, the neurodegeneration comprises amyotrophic lateral sclerosis (ALS), Huntington's Disease, Parkinson's Disease, Alzheimer's Disease, cognitive impairment, or a combination thereof, or other disorders involving microglia activation and/or neuroflammation. In certain such embodiments, the neurodegeneration comprises Alzheimer's Disease, cognitive impairment, or a combination thereof. The Alzheimer's Disease may be mild or moderate, and it maybe early or late. The cognitive impairment may be mild or moderate. In some embodiments, the Alzheimer's Disease is prodromal. In some embodiments, the neurodegeneration comprises Alzheimer's Disease with cognitive impairment, such as early Alzheimer's Disease with mild cognitive impairment. As used herein, prodromal Alzheimer's Disease is characterized by mild cognitive impairment due to Alzheimer's Disease plus very mild Alzheimer's Disease dementia, as described in Practical Neurology (June 2019) 36-47, which is hereby incorporated by reference herein in its entirety. Methods and standards for diagnosing and determining the different types, levels, and/or stages of Alzheimer's Disease, and the presence, absence, and/or level of cognitive impairment, are known to those of skill in the art (e.g., clinicians, physicians) and include, but are not limited to, those described in Practical Neurology (June 2019) 36-47, which is hereby incorporated by reference herein in its entirety.

In other aspects, the present disclosure provides methods of treating neurodegeneration, Alzheimer's disease and/or cognitive impairment in a human subject in need thereof (e.g., a subject with mild cognitive impairment and/or early Alzheimer's disease). In some such embodiments, the method comprises:
a) on day 1, administering to the subject a first daily dose that is a pharmaceutical composition comprising 90-180 mg of the compound of Formula I;
b) starting on day 2, administering to the subject a subsequent daily dose that is a pharmaceutical composition comprising 90-180 mg of the compound of Formula I; and
c) 6-28 days after the first daily dose, increasing the subsequent daily dose to a pharmaceutical composition comprising 135-270 mg of the compound of Formula I (i.e., increasing the subsequent daily dose to a pharmaceutical composition comprising 150-200% of the compound of Formula I in the subsequent daily dose administered in step b). As used herein, subsequent daily dosing “starting on day 2” repeats daily on day 3, day 4, day 5, day 6, day 7, day 8, and so on. As used herein, “6-28 days after the first daily dose” is day 7 to day 29, i.e., the subsequent daily dosing is increased starting on a day that is day 7, day 8, day 9, day 10, day 11, day 12, day 13, day 14, day 15, day 16, day 17, day 18, day 19, day 20, day 21, day 22, day 23, day 24, day 25, day 26, day 27, day 28, or day 29, to 135-270 mg of the compound of Formula I. In certain embodiments, pharmaceutical compositions comprising the compound of Formula I that may be suitable for use in the methods of the present disclosure include, but are not limited to, SDDs described in U.S. Pat. No. 10,526,318, which is incorporated herein by reference in its entirety. As described in U.S. Pat. No. 10,526,318, SDDs are produced by dissolving a compound of the invention (e.g., the compound of Formula I) and a pharmaceutically acceptable carrier (which may be a polymer, such as a cellulose derivative, such as hydroxypropyl methylcellulose acetate succinate (HPMCAS)) in an organic solvent or co-solvent mixture, then atomizing the solution into fine droplets in a drying chamber. The drying medium—typically heated nitrogen gas—evaporates the solvent, leaving the dry amorphous solid dispersion to be collected. Due to rapid solvent evaporation, SDDs achieve a thorough mixing of the compound of the invention and the carrier (e.g., the polymer carrier). The SDDs are also flowable and compressible, allowing them to be compressed, for example, into tablets for oral administration. HPMCAS have been demonstrated to be particularly effective in forming amorphous solid dispersions with poorly soluble active pharmaceutical ingredients such as cannabinoid receptor modulators that result in solubility enhancement through the ability to achieve and sustain a supersaturated solution of the active pharmaceutical ingredient. Curatolo et al., Pharmaceutical Research, 26(6), pp. 1419-1431 (2009). The extent of solubility enhancement and the sustainment is dependent on the acetate and succinate content of the polymer and varies depending on the specific active pharmaceutical ingredient being administered.

In some embodiments, the first daily dose is a pharmaceutical composition comprising 90-180 mg of the compound of Formula I, such as 112.5-180 mg, 135-180 mg, 157.5-180 mg, 180 mg, 90-157.5 mg, 112.5-157.5 mg, 135-157.5 mg, 157.5 mg, 90-135 mg, 112.5-135 mg, 135 mg, 90-112.5 mg, 112.5 mg, or 90 mg, of the compound of Formula I. Accordingly, in various embodiments, the pharmaceutical composition may comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, or 180 mg of the compound of Formula I, or any amount therebetween.

In some embodiments, the subsequent daily dose administered to the subject starting on day 2 is a pharmaceutical composition comprising 90-180 mg of the compound of Formula I, such as 112.5-180 mg, 135-180 mg, 157.5-180 mg, 180 mg, 90-157.5 mg, 112.5-157.5 mg, 135-157.5 mg, 157.5 mg, 90-135 mg, 112.5-135 mg, 135 mg, 90-112.5 mg, 112.5 mg, or 90 mg of the compound of Formula I. Accordingly, in various embodiments, the pharmaceutical composition may comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, or 180 mg of the compound of Formula I, or any amount therebetween.

In some embodiments, 6-28 days after the first daily dose (i.e., in step c), the subsequent daily dose is increased to a pharmaceutical composition comprising 135-270 mg of the compound of Formula I (i.e., 150-200% of the amount of the compound of Formula I in the subsequent daily dose administered in step b), such as 168.75-270 mg, 202.5-270 mg, 236.25-270 mg, 270 mg, 135-236.25 mg, 168.75-236.25 mg, 202.5-236.25 mg, 236.25 mg, 135-202.5 mg, 168.75-202.5 mg, 202.5 mg, 135-168.75 mg, 168.75 mg, or 135 mg of the compound of Formula I. Accordingly, in various embodiments, the pharmaceutical composition may comprise 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, or 270 mg of the compound of Formula I, or any amount therebetween. In certain embodiments, step c is performed 6 days after the first daily dose.

In some embodiments, the neurodegeneration comprises amyotrophic lateral sclerosis (ALS), Huntington's Disease, Parkinson's Disease, Alzheimer's Disease, cognitive impairment, or a combination thereof, or other disorders involving microglia activation and/or neuroflammation. In certain such embodiments, the neurodegeneration comprises Alzheimer's Disease, cognitive impairment, or a combination thereof. The Alzheimer's Disease may be mild or moderate, and it maybe early or late. The cognitive impairment may be mild or moderate. In some embodiments, the Alzheimer's Disease is prodromal. In some embodiments, the neurodegeneration comprises Alzheimer's Disease with cognitive impairment, such as early Alzheimer's Disease with mild cognitive impairment. As used herein, prodromal Alzheimer's Disease is characterized by mild cognitive impairment due to Alzheimer's Disease plus very mild Alzheimer's Disease dementia, as described in Practical Neurology (June 2019) 36-47, which is hereby incorporated by reference herein in its entirety. Methods and standards for diagnosing and determining the different types, levels, and/or stages of Alzheimer's Disease, and the presence, absence, and/or level of cognitive impairment, are known to those of skill in the art (e.g., clinicians, physicians) and include, but are not limited to, those described in Practical Neurology (June 2019) 36-47, which is hereby incorporated by reference herein in its entirety.

In still other aspects, the present disclosure provides methods of treating neurodegeneration, Alzheimer's disease and/or cognitive impairment in a human subject in need thereof (e.g., a subject with mild cognitive impairment and/or early Alzheimer's disease). In some such embodiments, the method comprises:
a) on day 1, administering to the subject a first daily dose that is a pharmaceutical composition comprising 90-180 mg of the compound of Formula I;
b) starting on day 2, administering to the subject a subsequent daily dose that is a pharmaceutical composition comprising 90-180 mg of the compound of Formula I; and
c) 6-28 days after the first daily dose, measuring the AUC of the compound of Formula I in the subject; and
d) if the AUC is less than 2000 h·ng/mL, increasing the subsequent daily dose by an amount corresponding to the percentage increase required to reach an AUC of at least 2000 h·ng/mL.

As used herein, subsequent daily dosing “starting on day 2” repeats daily on day 3, day 4, day 5, day 6, day 7, day 8, and so on.

As used herein, “6-28 days after the first daily dose” is day 7 to day 29, i.e., the AUC of the compound of Formula I is measured in the subject in step c on a day that is day 7, day 8, day 9, day 10, day 11, day 12, day 13, day 14, day 15, day 16, day 17, day 18, day 19, day 20, day 21, day 22, day 23, day 24, day 25, day 26, day 27, day 28, or day 29. In certain embodiments, the compound of Formula I is measured in the subject 6 days after the first daily dose, i.e., step c is performed 6 days after the first daily dose.

Methods to measure AUC of the compound of Formula I are known in the art; any suitable methods of measuring AUC may be used with the methods of the present disclosure. In certain embodiments, in step d, if the AUC is less than 2000 h·ng/mL, such as less than 1900 h·ng/mL, less than 1800 h·ng/mL, less than 1700 h·ng/mL, less than 1600 h·ng/mL, less than 1500 h·ng/mL, less than 1400 h·ng/mL, less than 1300 h·ng/mL, less than 1200 h·ng/mL, less than 1100 h·ng/mL, less than 1000 h·ng/mL, less than 900 h·ng/mL, or less than 800 h·ng/mL, the subsequent daily dose is increased by an amount corresponding to the percentage increase required to reach an AUC of at least that level. For example, if the AUC is less than 2000 h·ng/mL, the subsequent daily dose is increased by an amount corresponding to the percentage increase required to reach an AUC of at least 2000 h·ng/mL; if the AUC is less than 1200 h·ng/mL, the subsequent daily dose is increased by an amount corresponding to the percentage increase required to reach an AUC of at least 1200 h·ng/mL. In particular embodiments, in step d, if the AUC is less than 2000 h·ng/mL, the subsequent daily dose is increased by an amount corresponding to the percentage increase required to reach an AUC of at least 2000 h·ng/mL. In particular embodiments, in step d, if the AUC is less than 1900 h·ng/mL, the subsequent daily dose is increased by an amount corresponding to the percentage increase required to reach an AUC of at least 1900 h·ng/mL. In particular embodiments, in step d, if the AUC is less than 1800 h·ng/mL, the subsequent daily dose is increased by an amount corresponding to the percentage increase required to reach an AUC of at least 1800 h·ng/mL. In particular embodiments, in step d, if the AUC is less than 1700 h·ng/mL, the subsequent daily dose is increased by an amount corresponding to the percentage increase required to reach an AUC of at least 1700 h·ng/mL. In particular embodiments, in step d, if the AUC is less than 1600 h·ng/mL, the subsequent daily dose is increased by an amount corresponding to the percentage increase required to reach an AUC of at least 1600 h·ng/mL. In particular embodiments, in step d, if the AUC is less than 1500 h·ng/mL, the subsequent daily dose is increased by an amount corresponding to the percentage increase required to reach an AUC of at least 1500 h·ng/mL. In particular embodiments, in step d, if the AUC is less than 1400 h·ng/mL, the subsequent daily dose is increased by an amount corresponding to the percentage increase required to reach an AUC of at least 1400 h·ng/mL. In particular embodiments, in step d, if the AUC is less than 1300 h·ng/mL, the subsequent daily dose is increased by an amount corresponding to the percentage increase required to reach an AUC of at least 1300 h·ng/mL. In particular embodiments, in step d, if the AUC is less than 1200 h·ng/mL, the subsequent daily dose is increased by an amount corresponding to the percentage increase required to reach an AUC of at least 1200 h·ng/mL. In particular embodiments, in step d, if the AUC is less than 1100 h·ng/mL, the subsequent daily dose is increased by an amount corresponding to the percentage increase required to reach an AUC of at least 1100 h·ng/mL. In particular embodiments, in step d, if the AUC is less than 1000 h·ng/mL, the subsequent daily dose is increased by an amount corresponding to the percentage increase required to reach an AUC of at least 1000 h·ng/mL. In particular embodiments, in step d, if the AUC is less than 900 h·ng/mL, the subsequent daily dose is increased by an amount corresponding to the percentage increase required to reach an AUC of at least 900 h·ng/mL. In particular embodiments, in step d, if the AUC is less than 800 h·ng/mL, the subsequent daily dose is increased by an amount corresponding to the percentage increase required to reach an AUC of at least 800 h·ng/mL.

In certain embodiments, pharmaceutical compositions comprising the compound of Formula I that may be suitable for use in the methods of the present disclosure include, but are not limited to, SDDs described in U.S. Pat. No. 10,526,318, which is incorporated herein by reference in its entirety. As described in U.S. Pat. No. 10,526,318, SDDs are produced by dissolving a compound of the invention (e.g., the compound of Formula I) and a pharmaceutically acceptable carrier (which may be a polymer, such as a cellulose derivative, such as hydroxypropyl methylcellulose acetate succinate (HPMCAS)) in an organic solvent or co-solvent mixture, then atomizing the solution into fine droplets in a drying chamber. The drying medium—typically heated nitrogen gas—evaporates the solvent, leaving the dry amorphous solid dispersion to be collected. Due to rapid solvent evaporation, SDDs achieve a thorough mixing of the compound of the invention and the carrier (e.g., the polymer carrier). The SDDs are also flowable and compressible, allowing them to be compressed, for example, into tablets for oral administration. HPMCAS have been demonstrated to be particularly effective in forming amorphous solid dispersions with poorly soluble active pharmaceutical ingredients such as cannabinoid receptor modulators that result in solubility enhancement through the ability to achieve and sustain a supersaturated solution of the active pharmaceutical ingredient. Curatolo et al., Pharmaceutical Research, 26(6), pp. 1419-1431 (2009). The extent of solubility enhancement and the sustainment is dependent on the acetate and succinate content of the polymer and varies depending on the specific active pharmaceutical ingredient being administered.

In some embodiments, the first daily dose is a pharmaceutical composition comprising 90-180 mg of the compound of Formula I, such as 112.5-180 mg, 135-180 mg, 157.5-180 mg, 180 mg, 90-157.5 mg, 112.5-157.5 mg, 135-157.5 mg, 157.5 mg, 90-135 mg, 112.5-135 mg, 135 mg, 90-112.5 mg, 112.5 mg, or 90 mg, of the compound of Formula I. Accordingly, in various embodiments, the pharmaceutical composition may comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, or 180 mg of the compound of Formula I, or any amount therebetween.

In some embodiments, the subsequent daily dose administered to the subject starting on day 2 is a pharmaceutical composition comprising 90-180 mg of the compound of Formula I, such as 112.5-180 mg, 135-180 mg, 157.5-180 mg, 180 mg, 90-157.5 mg, 112.5-157.5 mg, 135-157.5 mg, 157.5 mg, 90-135 mg, 112.5-135 mg, 135 mg, 90-112.5 mg, 112.5 mg, or 90 mg of the compound of Formula I. Accordingly, in various embodiments, the pharmaceutical composition may comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, or 180 mg of the compound of Formula I, or any amount therebetween.

In some embodiments, the neurodegeneration comprises amyotrophic lateral sclerosis (ALS), Huntington's Disease, Parkinson's Disease, Alzheimer's Disease, cognitive impairment, or a combination thereof, or other disorders involving microglia activation and/or neuroflammation. In certain such embodiments, the neurodegeneration comprises Alzheimer's Disease, cognitive impairment, or a combination thereof. The Alzheimer's Disease may be mild or moderate, and it maybe early or late. The cognitive impairment may be mild or moderate. In some embodiments, the Alzheimer's Disease is prodromal. In some embodiments, the neurodegeneration comprises Alzheimer's Disease with cognitive impairment, such as early Alzheimer's Disease with mild cognitive impairment. As used herein, prodromal Alzheimer's Disease is characterized by mild cognitive impairment due to Alzheimer's Disease plus very mild Alzheimer's Disease dementia, as described in Practical Neurology (June 2019) 36-47, which is hereby incorporated by reference herein in its entirety. Methods and standards for diagnosing and determining the different types, levels, and/or stages of Alzheimer's Disease, and the presence, absence, and/or level of cognitive impairment, are known to those of skill in the art (e.g., clinicians, physicians) and include, but are not limited to, those described in Practical Neurology (June 2019) 36-47, which is hereby incorporated by reference herein in its entirety.

In still other aspects, the present disclosure provides methods of treating neurodegeneration, Alzheimer's disease and/or cognitive impairment in a human subject in need thereof (e.g., a subject with mild cognitive impairment and/or early Alzheimer's disease). In some such embodiments, the method comprises:
a) on day 1, administering to the subject a first daily dose that is a pharmaceutical composition comprising 90-180 mg of the compound of Formula I;
b) starting on day 2, administering to the subject a subsequent daily dose that is a pharmaceutical composition comprising 90-180 mg of the compound of Formula I;
c) 6-28 days after the first daily dose, increasing the subsequent daily dose to a pharmaceutical composition comprising 125-225% of the amount of the compound of Formula I in the subsequent daily dose administered in step b.

As used herein, subsequent daily dosing “starting on day 2” repeats daily on day 3, day 4, day 5, day 6, day 7, day 8, and so on. As used herein, “6-28 days after the first daily dose” is day 7 to day 29, i.e., the subsequent daily dosing is increased starting on a day that is day 7, day 8, day 9, day 10, day 11, day 12, day 13, day 14, day 15, day 16, day 17, day 18, day 19, day 20, day 21, day 22, day 23, day 24, day 25, day 26, day 27, day 28, or day 29, to 125-225% of the amount of the compound of Formula I in the subsequent daily dose administered in step b. In certain embodiments, pharmaceutical compositions comprising the compound of Formula I that may be suitable for use in the methods of the present disclosure include, but are not limited to, SDDs described in U.S. Pat. No. 10,526,318, which is incorporated herein by reference in its entirety. As described in U.S. Pat. No. 10,526,318, SDDs are produced by dissolving a compound of the invention (e.g., the compound of Formula I) and a pharmaceutically acceptable carrier (which may be a polymer, such as a cellulose derivative, such as hydroxypropyl methylcellulose acetate succinate (HPMCAS)) in an organic solvent or co-solvent mixture, then atomizing the solution into fine droplets in a drying chamber. The drying medium—typically heated nitrogen gas—evaporates the solvent, leaving the dry amorphous solid dispersion to be collected. Due to rapid solvent evaporation, SDDs achieve a thorough mixing of the compound of the invention and the carrier (e.g., the polymer carrier). The SDDs are also flowable and compressible, allowing them to be compressed, for example, into tablets for oral administration. HPMCAS have been demonstrated to be particularly effective in forming amorphous solid dispersions with poorly soluble active pharmaceutical ingredients such as cannabinoid receptor modulators that result in solubility enhancement through the ability to achieve and sustain a supersaturated solution of the active pharmaceutical ingredient. Curatolo et al., Pharmaceutical Research, 26(6), pp. 1419-1431 (2009). The extent of solubility enhancement and the sustainment is dependent on the acetate and succinate content of the polymer and varies depending on the specific active pharmaceutical ingredient being administered.

In some embodiments, the first daily dose is a pharmaceutical composition comprising 90-180 mg of the compound of Formula I, such as 112.5-180 mg, 135-180 mg, 157.5-180 mg, 180 mg, 90-157.5 mg, 112.5-157.5 mg, 135-157.5 mg, 157.5 mg, 90-135 mg, 112.5-135 mg, 135 mg, 90-112.5 mg, 112.5 mg, or 90 mg, of the compound of Formula I. Accordingly, in various embodiments, the pharmaceutical composition may comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, or 180 mg of the compound of Formula I, or any amount therebetween.

In some embodiments, the subsequent daily dose administered to the subject starting on day 2 is a pharmaceutical composition comprising 90-180 mg of the compound of Formula I, such as 112.5-180 mg, 135-180 mg, 157.5-180 mg, 180 mg, 90-157.5 mg, 112.5-157.5 mg, 135-157.5 mg, 157.5 mg, 90-135 mg, 112.5-135 mg, 135 mg, 90-112.5 mg, 112.5 mg, or 90 mg of the compound of Formula I. Accordingly, in various embodiments, the pharmaceutical composition may comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, or 180 mg of the compound of Formula I, or any amount therebetween.

In some embodiments, 6-28 days after the first daily dose (i.e., in step c), the subsequent daily dose is increased to a pharmaceutical composition comprising 125-225% of the amount of the compound of Formula I in the subsequent daily dose administered in step b, such as 150-225%, 175-225%, 200-225%, 225%, 125-200%, 150-200%, 175-200%, 200%, 125-175%, 150-175%, 175%, 125-150%, 150%, or 125% of the amount of the compound of Formula I in the subsequent daily dose administered in step b. Accordingly, in various embodiments, the pharmaceutical composition in step c may comprise 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, or 225% of the amount of the compound of Formula I in the subsequent daily dose administered in step b, or any amount therebetween.

In some embodiments, the neurodegeneration comprises amyotrophic lateral sclerosis (ALS), Huntington's Disease, Parkinson's Disease, Alzheimer's Disease, cognitive impairment, or a combination thereof, or other disorders involving microglia activation and/or neuroflammation. In certain such embodiments, the neurodegeneration comprises Alzheimer's Disease, cognitive impairment, or a combination thereof. The Alzheimer's Disease may be mild or moderate, and it maybe early or late. The cognitive impairment may be mild or moderate. In some embodiments, the Alzheimer's Disease is prodromal. In some embodiments, the neurodegeneration comprises Alzheimer's Disease with cognitive impairment, such as early Alzheimer's Disease with mild cognitive impairment. As used herein, prodromal Alzheimer's Disease is characterized by mild cognitive impairment due to Alzheimer's Disease plus very mild Alzheimer's Disease dementia, as described in Practical Neurology (June 2019) 36-47, which is hereby incorporated by reference herein in its entirety. Methods and standards for diagnosing and determining the different types, levels, and/or stages of Alzheimer's Disease, and the presence, absence, and/or level of cognitive impairment, are known to those of skill in the art (e.g., clinicians, physicians) and include, but are not limited to, those described in Practical Neurology (June 2019) 36-47, which is hereby incorporated by reference herein in its entirety.

### Compositions and Modes of Administration

The compound of this invention may be used in treating the conditions described herein, in the form of the free base, salts (preferably pharmaceutically acceptable salts), solvates, hydrates, prodrugs, isomers, or mixtures thereof. All forms are within the scope of the disclosure. Acid addition salts may be formed and provide a more convenient form for use; in practice, use of the salt form inherently amounts to use of the base form. The acids which can be used to prepare the acid addition salts include preferably those which produce, when combined with the free base, pharmaceutically acceptable salts, that is, salts whose anions are non-toxic to the subject organism in pharmaceutical doses of the salts, so that the beneficial properties inherent in the free base are not vitiated by side effects ascribable to the anions. Although pharmaceutically acceptable salts of the basic compounds are preferred, all acid addition salts are useful as sources of the free base form even if the particular salt per se is desired only as an intermediate product as, for example, when the salt is formed only for the purposes of purification and identification, or when it is used as an intermediate in preparing a pharmaceutically acceptable salt by ion exchange procedures.

Pharmaceutically acceptable salts within the scope of the disclosure include those derived from the following acids; mineral acids such as hydrochloric acid, sulfuric acid, phosphoric acid and sulfamic acid; and organic acids such as acetic acid, citric acid, lactic acid, tartaric acid, malonic acid, methanesulfonic acid, ethanesulfonic acid, benzenesulfonic acid, p-toluenesulfonic acid, cyclohexylsulfamic acid, quinic acid, and the like.

The compound of the present invention can be formulated as pharmaceutical compositions and administered to a subject in need of treatment, for example a mammal, such as a human patient, in a variety of forms adapted to the chosen route of administration, for example, orally, nasally, intraperitoneally, or parenterally (e.g., by intravenous, intraperitoneal, subcutaneous, intramuscular, transepithelial, nasal, intrapulmonary, intrathecal, rectal or topical routes). Parenteral administration may be by continuous infusion over a selected period of time.

In accordance with the methods of the disclosure, the described compound may be administered to a patient in a variety of forms depending on the selected route of administration, as will be understood by those skilled in the art. The compositions containing the compound of the disclosure can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

A composition comprising a compound of the present disclosure may also contain adjuvants, such as preservatives, wetting agents, emulsifying agents and dispersing agents.

Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

A person skilled in the art would know how to prepare suitable formulations. Conventional procedures and ingredients for the selection and preparation of suitable formulations are described, for example, in Remington's Pharmaceutical Sciences (1990—18^{th} edition) and in The United States Pharmacopeia: The National Formulary (USP 24 NF19) published in 1999.

Thus, the compound of the invention may be systemically administered, e.g., orally, in combination with a pharmaceutically acceptable vehicle such as an inert diluent or an assimilable edible carrier; or by inhalation or insufflation. They may be enclosed in hard or soft shell gelatin capsules, may be compressed into tablets, or may be incorporated directly with the food of the patient's diet. For oral therapeutic administration, the compounds may be combined with one or more excipients and used in the form of ingestible tablets, buccal tablets, troches, capsules, elixirs, suspensions, syrups, wafers, and the like. The compounds may be combined with a fine inert powdered carrier and inhaled by the subject or insufflated. Such compositions and preparations should contain at least 0.1% of the compound of formula I. The percentage of the compositions and preparations may, of course, be varied and may conveniently be between about 2% to about 60% of the weight of a given unit dosage form. The amount of the compounds in such therapeutically useful compositions is such that an effective dosage level will be obtained.

In certain embodiments of the disclosure, compositions comprising a compound of the present disclosure for oral administration include capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and the like, each containing a predetermined amount of the compound of the present disclosure as an active ingredient.

In solid dosage forms for oral administration (capsules, tablets, troches, pills, dragees, powders, granules, and the like), one or more compositions comprising the compound of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, gum tragacanth, corn starch, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, the compounds may be incorporated into sustained-release preparations and devices. For example, the compounds may be incorporated into time release capsules, time release tablets, and time release pills.

Liquid dosage forms for oral administration include pharmaceutically acceptable emulsions, microemulsions, solutions, suspensions, syrups, and elixirs. In addition to the compound of the present disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, salts and/or prodrugs thereof, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In certain embodiments, pharmaceutical compositions suitable for parenteral administration may comprise the compound of the present disclosure in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compounds may be administered intravenously or Intraperitoneally by infusion or injection. Solutions of the compounds or their salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the compounds which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the compounds in the required amount in the appropriate solvent with various of the other ingredients enumerated above, as required, followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

The compounds may conveniently be presented in a single dose or as divided doses administered at appropriate intervals, for example, as two, three, four or more sub-doses per day. The sub-dose itself may be further divided, e.g., into a number of discrete loosely spaced administrations.

The compounds and/or compositions of the disclosure can be used alone or conjointly with other therapeutic agents, or in combination with other types of treatment for treating neurodegeneration. For example, these other therapeutically useful agents may be administered in a single formulation, simultaneously or sequentially with the compound of the present disclosure according to the methods of the disclosure.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The articles “a” and “an” are used herein to refer to one or to more than one (i.e., to at least one) of the grammatical object of the article unless context clearly indicates that the article used refers to only a single grammatical object. By way of example, “an element” means one element or more than one element unless context dictates otherwise.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4^{th} ed.”, W. H. Freeman & Co., New York (2000); Griffiths et al., “Introduction to Genetic Analysis, 7^{th} ed.”, W. H. Freeman & Co., N.Y. (1999); and Gilbert et al., “Developmental Biology, 6^{th} ed.”, Sinauer Associates, Inc., Sunderland, MA (2000).

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compound represented by formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of the compound of formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of the compound of formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

All of the above, and any other publications, patents and published patent applications referred to in this application are specifically incorporated by reference herein. In case of conflict, the present specification, including its specific definitions, will control.

### EXAMPLES

The invention now being generally described, it will be more readily understood by reference to the following examples, which are included merely for purposes of illustration of certain aspects and embodiments of the present invention, and are not intended to limit the invention.

### Reference Example 1: Synthesis of Compound of Formula I

The synthesis of the compound of Formula I described in U.S. Pat. No. 8,440,832, which is incorporated herein by reference; the synthesis as described therein is reproduced below.

### Step 1. 4-Iodo-3-(2-methyl-allyloxy)-benzoic acid methyl ester

To a solution of methyl 3-hydroxy-4-iodobenzoate (1.5 g, 5.4 mmol) in anhydrous methyl ethyl ketone (60 mL) was added finely powdered Potassium carbonate (1.49 g, 10.78 mmol) followed by 3-bromo-2-methyl-propene (0.81 mL, 1.1 g, 8.15 mmol). The reaction mixture was heated at 70° C. for 4 h. The mixture was diluted filtrated, washed with water and dried over MgSO₄. Evaporation of the solvent and of the remaining bromopropene in vacuo afforded the requisite alkylated ester as a yellow oil. M: 1.4 g, Yield: 78%

NMR (CDCl₃, 1H): 1.90 (3H, d, J=1.2 Hz), 194 (3H, s), 4.56 (2H, s), 5.06 (1H, d, J=1.2 Hz), 5.25 (1H, d, J=1.2 Hz), 7.38 (1H, dd, J=8.1 Hz, J=1.8 Hz), 7.44 (1H, d, J=1.8 Hz), 7.88 (1H, d, J=1.8 Hz)

### Step 2. 3-Benzyl-3-methyl-2,3-dihydro-benzofuran-6-carboxylic acid methyl ester

To a solution of 4-Iodo-3-(2-methyl-allyloxy)-benzoic acid methyl ester (455 mg, 1.37 mmol) obtained in Example 4(A), in DMF (15 mL) were added Potassium carbonate (379 mg, 2.74 mmol), Tetrabutylammonium chloride (380 mg, 1.37 mmol), Palladium acetate (25.6 mg, 0.136 mmol) in DMF (5 mL) and Phenylboronic acid (200 mg, 1.64 mmol). The resulting mixture was stirred for 3 hat 115° C., cooled to room temperature, filtered over silica, washed with water, dried over MgSO₄ and concentrated. Column chromatography (silica gel, heptane/CH2C12: 4/6) afforded 368 mg (95%) of the title compound as a slightly brown oil which crystallize. Mp: 52° C.

NMR (CDCl₃, 1H): 1.38 (3H, s), 2.86 (1H, d, J=14 Hz), 2.93 (1H, d, J=14 Hz), 3.89 (3H, s), 4.12 (1H, d, J=8.7 Hz), 4.55 (1H, d, J=8.7 Hz), 6.93-6.98 (3H, m), 7.22-7.24 (3H, m), 7.38 (1H, d, J=1.2 Hz), 7.59 (1H, dd, J1=7.5 Hz, J2=1.2 Hz).

### Step 3. 3-Benzyl-3-methyl-2,3-dihydro-benzofuran-6-carboxylic acid

A mixture of 3-Benzyl-3-methyl-2,3-dihydro-benzofuran-6-carboxylic acid methyl ester (300 mg, 1.06 mmol), sodium hydroxide (260 mg, 6.5 mmol), ethanol (10 ml) and water (1 ml) in tetrahydrofuran (10 ml), is stirred for 12 h at room temperature. The reaction medium is acidified by adding a 1.2 M hydrochloric acid solution and extracted with ethyl acetate. The organic phase is washed with water, dried (Na2SO4), and concentrated in a rotary evaporator. The product is obtained as a white solid (300 mg, 100%). Mp: 165° C.

NMR (CDCl₃, ¹H): 1.39 (3H, s), 2.87 (1H, d, J=14 Hz), 2.93 (1H, d, J=14 Hz), 4.14 (1H, d, J=8.7 Hz), 4.57 (1H, d, J=8.7 Hz), 6.96-7.00 (3H, m), 7.22-7.25 (3H, m), 7.45 (1H, d, J=1.2 Hz), 7.65 (1H, dd, J1=7.8 Hz, J2=1.2 Hz).

### Step 4. 3-benzyl-3-methyl-2,3-dihydrobenzofuran-6-carboxylic acid-piperidine amide

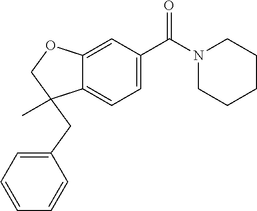 [SMILES: CC1(Cc2ccccc2)COc2cc(C(=O)N3CCCCC3)ccc21]

To a stirred suspension of the 3-Benzyl-3-methyl-2,3-dihydrobenzofuran-6-carboxylic acid (80 mg, 0.3 mmol) previously obtained and Piperidine (28 mg, 33 μL, 0.33 mmol) in dichloromethane (3 mL) and DMF (2 mL) were added 0-(7-Azabenzotriazol-1-yl)-N,N,N′,N′-tetramethyluronium hexafluorophosphate (HATU) (125 mg, 0.33 mmol) and then a solution of N,N-diisopropylethylamine (58 mg, 78 μL, 0.45 mmol, mL) in DMF (1 mL). The reaction mixture was stirred at ambient temperature for 18 h. The reaction medium is acidified by adding a 1.2 M hydrochloric acid solution and extracted with ethyl acetate. The organic phase is washed with water, dried (MgSO₄), and concentrated to give the amide which is purified by flash chromatography (AcOEt/heptane: 4/6) to afford 50 mg of a white solid (yield: 50%).

NMR (CDCl₃, ¹H): 1.36 (3H, s), 1.54-1.67 (6H, m), 2.85 (1H, d, J=13.2 Hz), 2.90 (1H, d, J=13.2 Hz), 3.35 (2H, m), 3.68 (2H, m), 4.09 (1H, d, J=8.7 Hz), 4.53 (1H, d, J=8.7 Hz), 6.75 (1H, m), 6.88 (1H, dd, 31=7.5 Hz, J2=1.2 Hz), 6.94 (1H, d, J=7.5 Hz), 7.00 (2H, m), 7.21-7.24 (3H, m).

### Example 2: A Phase 1b Double Blind Multiple Ascending Dose Study of the Safety and Pharmacokinetics of NTRX-07 in Normal Volunteers and Patients with Mild Cognitive Impairment or Early Alzheimer's Disease

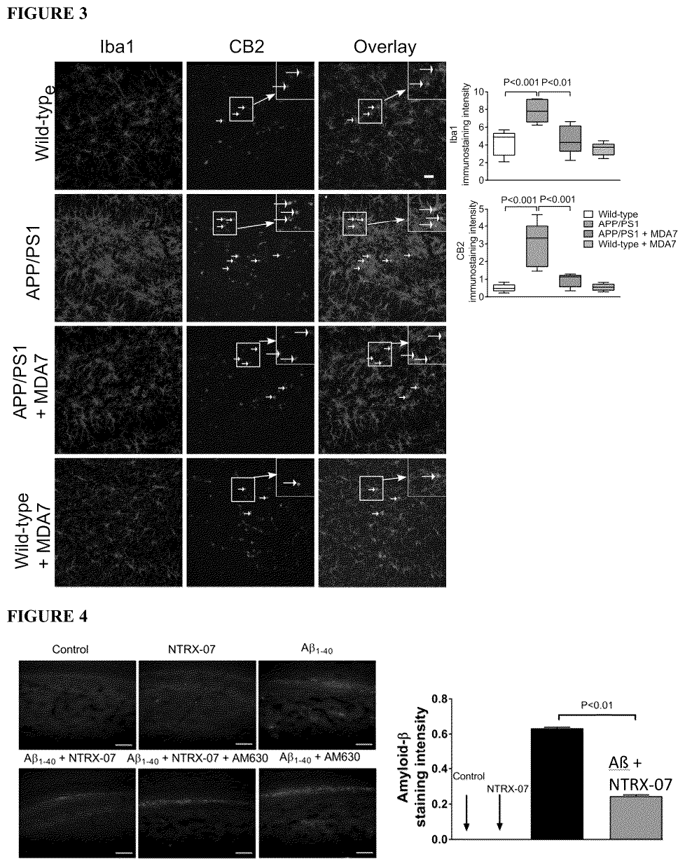

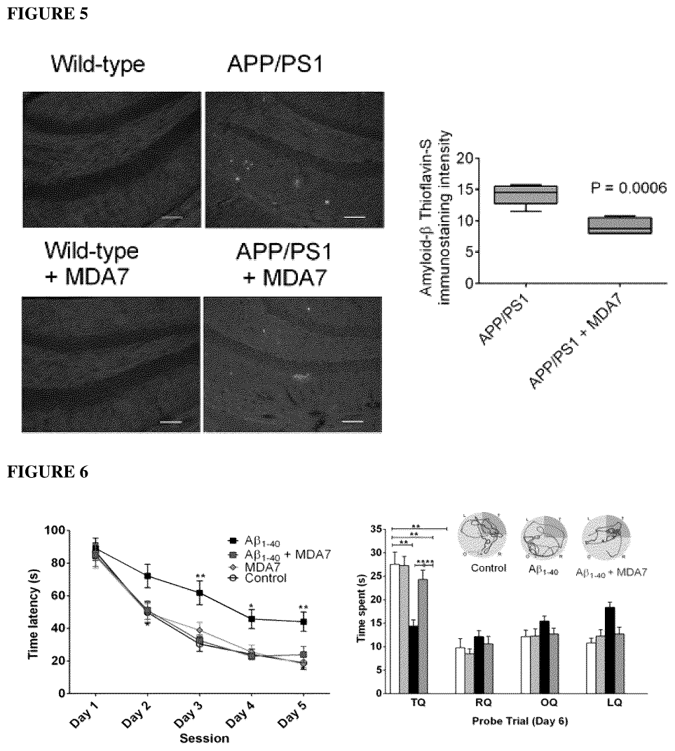

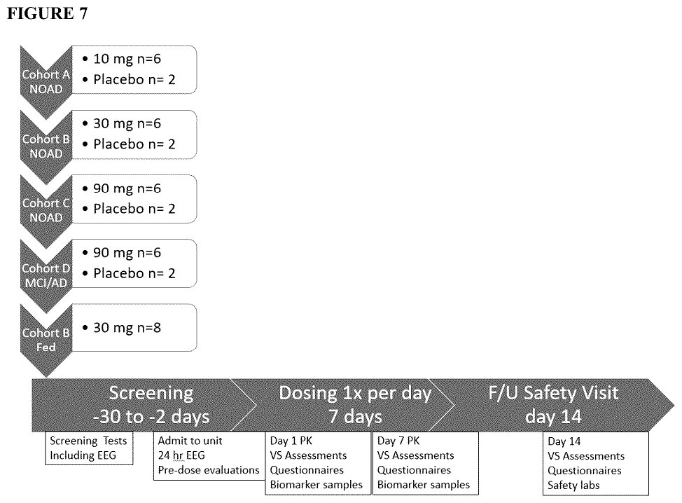

Background: NTRX-07 is an orally administered, brain-permeable, highly selective cannabinoid receptor type 2 (CBR2) agonist under development for the treatment of neuroinflammatory-related diseases, including Alzheimer's Disease (AD). Microglial-mediated neuroinflammation has been identified in the cascade, leading to neuronal death (FIG. 2). Chronic activation of microglia causes neuronal damage, and chronically activated microglia express cannabinoid 2 (CB2) receptors. Targeting the CB2 receptor has been shown to decrease neuroinflammation and resulting neuronal damage. Preclinical studies of NTRX-07 in AD models have demonstrated decreased inflammatory changes in the brain, improved clearance of A-beta proteins, improved long-term potentiation, and improved learning and memory in rodent models (FIGS. 3-6). Wu J. et al., Eur. J. Pharmacol. 811:12-20 (2017) doi: 10.1016/j.ejphar.2017.05.044. A previous Phase 1a study of NTRX-07 demonstrated no major adverse effects with single doses up to 2 mg/kg in healthy volunteers. Foss J, Naguib M, Giordano T. 2020. Alzheimer's Dementia. doi: 10.1002/alz.039150. Doses of 3.5 and 4 mg/kg were associated with transient lightheadedness. The objectives of the present study were to study the safety, tolerability, and pharmacokinetics (PK) of repeat dosing in older volunteers and a cohort of subjects with AD. Exploratory endpoints included a food effect cohort, food effect on PK, EEG and plasma biomarkers of inflammation.

Methods: All study procedures were approved by the IRB, and informed consent was obtained. Three cohorts of volunteers 45-80 years of age with well-controlled comorbidities (NOAD) and one cohort of AD patients diagnosed with cognitive impairment consistent with prodromal AD per International Working Group criteria or mild AD per National Institute on Aging—Alzheimer's Association criteria (n=6 active, 2 placebo per cohort) were enrolled. Participants were admitted to the study site for the duration of the dosing and returned 7-12 days after the last dose for a safety visit. Participants received NTRX-07 (10, 30, or 90 mg) or a placebo in a double-blinded randomization orally once daily for seven days. Subjects were assessed for changes in vital signs, including maneuvers to induce lightheadedness, electrocardiograms, 24-hour electroencephalograms (EEG), and safety laboratory studies. The second cohort returned and received a single repeat dose after a standard high-fat meal. The AD cohort also underwent cognitive testing and had blood samples for biomarkers drawn. Subjects had PK sampling done after the first and final dose of the study drug. An independent safety review committee conducted a blinded safety review of adverse events and plasma levels between each cohort.

Assessments: Subjects were assessed for changes in vital signs, orthostatic blood pressure, Romberg test, nystagmus, timed get up and go, electrocardiograms, electroencephalograms (EEG), and laboratory studies. Subjective questionnaires of drug effects (ARCI-M) were administered. Cohort B returned and received a single repeat dose after a standard high-fat meal. The AD cohort also underwent cognitive testing (MMSE, ADAS-COG, MOCA) and had blood samples for biomarkers drawn.

All subjects had PK sampling done after the first and final dose of the study drug.

Protocol-specified EEG assessments were for safety assessment of any potential epileptogenic changes. A post-hoc analysis of quantitative EEG (qEEG) changes was also performed.

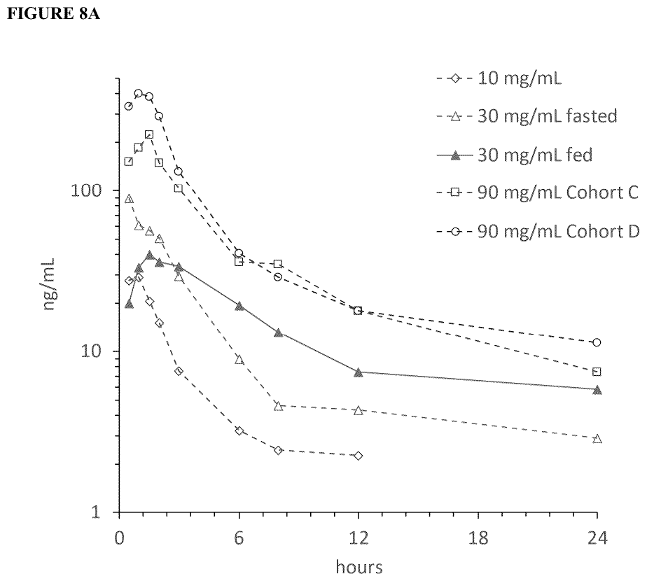

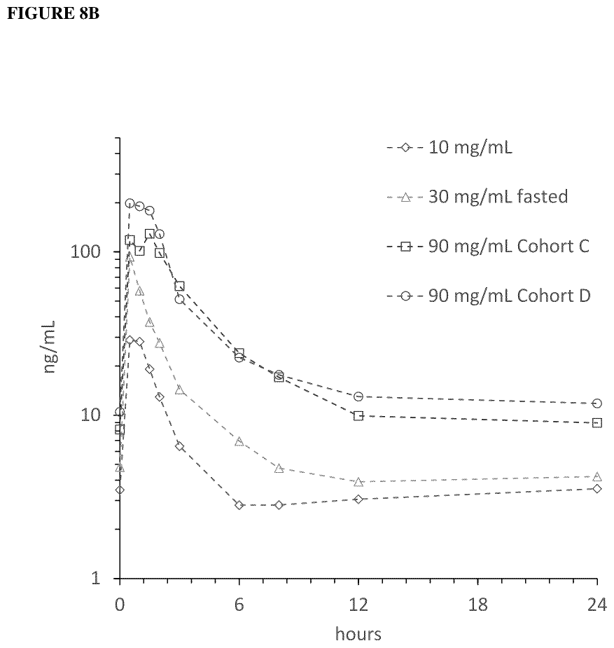

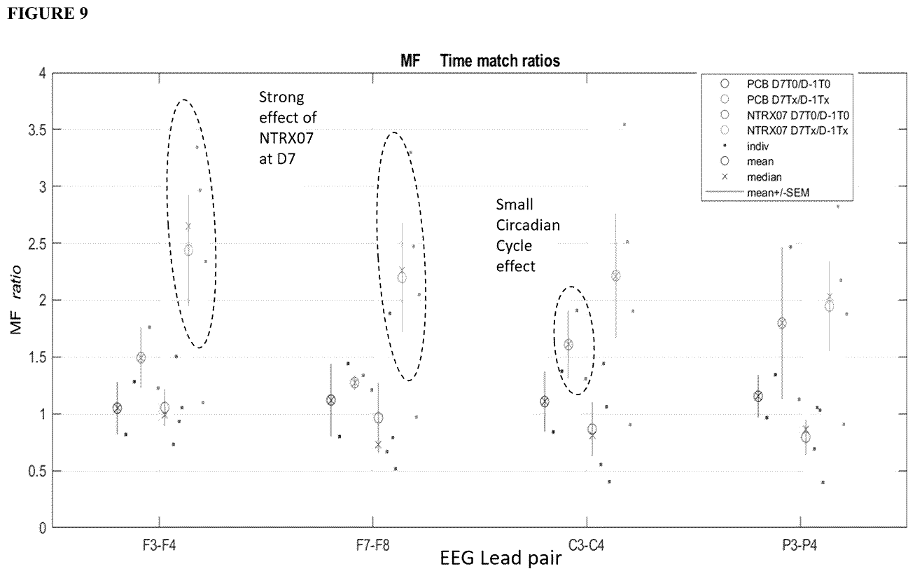

Results: There were no dose-limiting or serious adverse events during the trial (Table 2). One subject withdrew from the trial due to social reasons. Five participants had orthostatic changes in blood pressure, generally observed early in dosing (Table 1). Three episodes were asymptomatic, and none required treatment. No participants had changes in the timed get-up and go, Nystagmus test, or Romberg test. No participants had abnormalities on the EEG. A change in qEEG was observed in AD patients compared to non-AD patients, consistent with what has been reported, and treatment partially reversed this AD effect (FIG. 9). No clinically meaningful changes in ECG or safety labs were observed. PK at the high dose was similar between Non-AD and AD participants (Table 4, FIGS. 8A-8B) with an average of AUC0-24 h (h·ng/mL) of 1291 and 1556, and Cmax (ng/ml) of 439 and 477, respectively. A decrease of levels at Day 7 was observed, suggesting a change in clearance and suggesting hepatic enzyme induction. The high-fat meal increased observed plasma levels, decreased Cmax, but AUC was comparable. No significant changes in cognitive scores were observed (Table 3), though there was an interesting trend towards improvement in the AD participants. No significant changes in biomarkers or plasma biomarkers were observed.

TABLE 1
Orthostatic events observed during trial

| Cohort | Subject ID | Treatment | Notes | Treatment | Severity |
|---|---|---|---|---|---|
| B | 18 | NTRX-07 | Asymptomatic | No | Mild |
| C | 26 | NTRX-07 | Lightheadedness; | No | Mild |
| | | | weakness; | | |
| | | | presyncope | | |
| C | 31 | Placebo | Asymptomatic; | No | Mild |
| | | | 2 events | | |
| C | 39 | NTRX-07 | Asymptomatic | No | Mild |
| D | 56 | NTRX-07 | Symptomatic | No | Mild |

TABLE 2
Adverse events observed during trial

| | | | | | | Related |
|---|---|---|---|---|---|---|
| | | | Description of | | | to Study |
| Cohort | Subject ID | Treatment | Event | Treated? | Severity | Drug? |
| C | 16 | NTRX-07 | Presyncope | No | Mild | Possible |
| | | | Weakness | No | Mild | Possible |
| C | 31 | Placebo | Hypotension | No | Mild | Possible |
| C | 34 | NTRX-07 | Subfebrility | No | Mild | Possible |
| | | | Heartburn | No | Mild | Possible |
| C | 39 | NTRX-07 | Dry mouth | No | Mild | Probable |
| D | 46 | NTRX-07 | Xerostomia | No | Mild | Possible |
| | | | Hypertension | Yes | Moderate | None |
| D | 48 | NTRX-07 | Nausea | No | Mild | Possible |
| D | 50 | Placebo | Hypertension | Yes | Moderate | None |
| | | | Blurred vision | No | Mild | None |
| D | 51 | NTRX-07 | Hypertension, | Yes | Moderate | None |
| | | | tachycardia | | | |
| D | 52 | NTRX-07 | Dizziness | No | Mild | Possible |

TABLE 3
MOCA and ADAS-COG after 7 days of NTRX-07

| | MOCA | | | ADAS-COG | | |
|---|---|---|---|---|---|---|
| | | | EOS Visit | | | EOS Visit |
| | DAY −2 | DAY 8 | (DAY 14-19) | DAY −2 | DAY 8 | (DAY 14-19) |
| Placebo | | | | | | |
| 042 | 15 | 22 | 19 | 13.00 | 13.00 | 16.33 |
| 050 | 14 | 15 | 13 | 12.67 | 16.00 | 17.33 |
| NTRX-07 | | | | | | |
| 046 | 22 | 22 | 25 | 17.00 | 11.00 | 9.33 |
| 048 | 21 | 21 | 15 | 18.33 | 15.33 | 19.33 |
| 051 | 21 | 19 | 20 | 13.33 | 11.67 | 11.00 |
| 052 | 24 | 20 | 23 | 22.00 | 12.67 | 12.00 |
| 056 | 18 | 19 | 22 | 18.00 | 12.00 | 13.00 |
| 058 | 17 | 17 | 19 | 16.33 | 13.67 | 14.33 |

TABLE 4
Pharmacokinetic Parameters after 7 days of NTRX-07

| | Average of | Average of | | | |
|---|---|---|---|---|---|
| Cohort/Day of | AUC24 h | Cₘₐₓ | Average of | Average of | Average of |
| Treatment | (h · ng/mL) | (ng/mL) | Tmax (h) | Cl/F (L/h) | Vd/F (L) |
| Cohort A | | | | | |
| Day 1 | 78.8 ± 43.7 | 35.6 ± 12.8 | 0.7 ± 0.3 | 174 ± 108 | 1279 ± 544 |
| Day 7 | 90.4 ± 67.9 | 37.7 ± 15.9 | 0.8 ± 0.3 | 151 ± 74 | 307 ± 126 |
| Cohort B | | | | | |
| Day 1 | 328 ± 213 | 109 ± 58 | 0.9 ± 0.5 | 137 ± 99 | 349 ± 175 |
| Day 7 | 236 ± 186 | 120 ± 79 | 0.6 ± 0.2 | 206 ± 176 | 391 ± 235 |
| Fed | 390 ± 280 | 60 ± 37 | 2.4 ± 1.8 | 123 ± 94 | 699 ± 424 |
| Cohort C | | | | | |
| Day 1 | 1267 ± 509 | 400 ± 202 | 2.3 ± 2.2 | 83 ± 36 | 337 ± 316 |
| Day 7 | 741 ± 357 | 189 ± 95 | 1.3 ± 1.0 | 149 ± 76 | 5568 ± 231 |
| Cohort D | | | | | |
| Day 1 | 1556 ± 605 | 478 ± 154 | 1.1 ± 0.6 | 62 ± 19 | 211 ± 90 |
| Day 7 | 806 ± 196 | 240 ± 38 | 1.1 ± 0.5 | 117 ± 28 | 384 ± 69 |

TABLE 5
Plasma Biomarkers of Inflammation

| | | Day 1 | | | Day 8 | | |
|---|---|---|---|---|---|---|---|
| Biomarkers | n | Median(IQR) | | n | Median(IQR) | | P |
| IL-12p70(pg/ml) | 5 | 0.34 | (0.22-0.36 ) | 6 | 0.33 | (0.2-0.4) | 0.733 |
| IL-1β(pg/ml) | 6 | 0.13 | (0.09-0.17) | 3 | 0.11 | (0.05-0.44 ) | 0.386 |
| IL-4(pg/ml) | 2 | 1.38 | (1.1-1.67 ) | 2 | 1.14 | (0.91-1.37 ) | 0.001* |
| IL-5(pg/ml) | 5 | 0.17 | (0.1-0.3) | 6 | 0.19 | (0.11-0.27 ) | 0.302 |
| INFg(pg/ml) | 6 | 0.08 | (0.06-0.15 ) | 6 | 0.08 | (0.06-0.12 ) | 0.940 |
| IL-6(pg/ml) | 6 | 2.19 | (1.03-3.27 ) | 6 | 2.44 | (1.82-3.73 ) | 0.663 |
| IL-8(pg/ml) | 6 | 7.65 | (5.46-8.54 ) | 6 | 10.41 | (6.28-11.5) | 0.162 |
| IL-22(pg/ml) | 6 | 0.9 | (0.52-2.61 ) | 6 | 0.87 | (0.57-1.02 ) | 0.381 |
| TNFa(pg/ml) | 5 | 0.69 | (0.66-0.86 ) | 5 | 0.83 | (0.69-1.04 ) | 0.463 |
| IL-10(pg/ml) | 6 | 0.35 | (0.29-0.57 ) | 6 | 0.38 | (0.24-0.6 ) | 0.476 |
| hCRP(ng/ml) | 6 | 4135.17 | (359.49-7067.17 ) | 6 | 4211.88 | (512.84-6887.35 ) | 0.854 |
| Chitinase-3(pg/ml) | 6 | 43668.33 | (35077.33-54239 ) | 6 | 40345.17 | (34593.67-49559 ) | 0.054 |
| TREM-2 (pg/ml) | 6 | 38468.67 | (30025-42188 ) | 6 | 39576.17 | (30447.67-61696 ) | 0.065 |
| IL-2(pg/ml) | 2 | 0.16 | (0-0.33 ) | 3 | 0 | (0-0.47 ) | 0.869 |
| Neurogranin(pg/ml) | 4 | 671.19 | (614.7-774.08 ) | 3 | 881.02 | (670.23-1285.09 ) | 0.107 |
| Total Tau(pg/ml) | 6 | 2.07 | (1.65-2.59 ) | 5 | 2.03 | (1.93-2.28 ) | 0.642 |
| AB42(pg/ml) | 6 | 8.29 | (7.35-10.84 ) | 5 | 8.57 | (8.33-8.77 ) | 0.303 |
| AB40(pg/ml) | 6 | 232.84 | (217.73-293.76 ) | 5 | 226.52 | (215.35-241.77 ) | 0.159 |
| NF-LIGHT(pg/ml) | 6 | 18.24 | (16.73-21.51 ) | 6 | 18.49 | (16.3-19.34 ) | 0.610 |
| pTau-181(pg/ml) | 6 | 21.53 | (15.09-26.07 ) | 6 | 20.16 | (18.47-26.95 ) | 0.584 |

Visual inspection of changes in potential inflammatory biomarkers was notable for wide variation in baseline values.

Individual subjects had changes from baseline in individual biomarkers, with some increasing and some decreasing, but no consistent changes across any set of biomarkers studied were observed.

Conclusions: NTRX-07 was safely administered for 7 days at doses up to 90 mg/day. The primary side effect observed was mild transient orthostatic changes in blood pressure, usually with early doses. Plasma levels were within the target ranges based on the allometric scaling of preclinical data. Future studies of NTRX-07 in the AD population to determine the effect on biomarkers and cognitive effects are planned.

### Example 3: Additional Phase 1b Trial Results

### Executive Summary

Phase 1a, single ascending dose study completed in 2020
   Better drug exposure in blood than observed in rodents or dogs
   No adverse events level (2 mg/kg) provides blood levels equivalent to those that were efficacious in animal models of AD
   Only minor, non-dose limiting adverse events (lightheadedness and flushing, coded as vertigo)
Phase 1b, multiple ascending dose study carried out in Hungary by CRU Global competed in June 2023
   No dose limiting toxicities at any of the tested doses
   Blood levels equivalent to those that were efficacious in animal models of AD achieved at the 90 mg dose
   Trend towards improvement in EEG and ADAS-COG in AD cohort
Submitted Phase 2a protocol to European regulators in Hungary for initial scientific review
   Trial design acceptable
   Minor additional metabolite analysis suggested

Table 6 below summarizes issues raised by the US FDA prior to the Phase 1b study:

| Issue | Reasons | Assessment | Outcome in Phase 1b |
|---|---|---|---|
| Vertigo | Subjects at 2 highest | 3 tests for vertigo administered | No evidence of |
| | dose groups in Phase 1a | while patient confined in unit | vertigo with any test |
| | had dizziness, reported | during testing | |
| | by site as vertigo | | |
| CNS | Seizures in 1 or 2 dogs | 24 hour EEG readings for | No change in EEG at |
| Safety | at 200 mg/kg/day | safety, analyzed by Biotrial, a | any dose |
| | | company who has used this | |
| | | outcome for CNS safety | |
| | | assessments | |

### Phase 1b MAD Study Design

This study was carried out in Hungary under regulatory approval from the European Union to address the concerns raised by the FDA.

An informational letter was filed with the FDA to inform them of this study. The study was designed as follows:
3 healthy cohorts (cohorts A-C) followed by AD cohort (cohort D) treated at MTD
6 treated, 2 placebos in each cohort
Doses: 10, 30, 90 mg/treatment—latter doses can be adjusted based on PK assessment
In unit, 7 days of treatment
   Monitor vertigo with various tests: (1) timed get-up and go, (2) Nystagmus test, (3) Romberg test, and (4) changes in orthostatic blood pressure (Day 1, 3 and 8)
   Use 24 hr EEG monitoring for CNS safety assessment
   Assess plasma biomarkers of inflammation and AD pre- and post-treatment in AD cohort
   Assess changes on cognition post-treatment using MMSE and ADAS-cog
Fed/fasted study to determine food effects on PK as a subset of the Phase 1b

### Results

There was no evidence of vertigo at any dose in timed get-up and go, Nystagmus test, or Romberg test. Treatment emergent changes in orthostatic blood pressure are shown in Table 1.

Additionally, there was no evidence of abnormal brain activity. No subjects had clinically meaningful abnormal values or changes from baseline, nor differences between placebo and active drug, in EEG readings in any of the 4 cohorts. Analysis of EEG scans for suggestions of cognitive changes by qEEG analysis was done using a small sample size, thus no formal statistical assessment was done. A difference between healthy and AD groups was observed at baseline, and there was a partial reversion of AD effect by NTRX-07. The qEEG post hoc exploratory analysis of NTRX-07 was completed as outlined below:
24-hour EEG was performed as a safety assessment in C102—no evidence of abnormal activity was observed
Quantitative EEG (qEEG) examines the power spectrum of the EEG across several frequencies
qEEG has been demonstrated to be a sensitive and specific diagnostic tool in AD, and has been studied as a biomarker for drug effects in AD and other neurological diseases.
An EXPLORATORY Post Hoc analysis of qEEG extracted from the 24-hour EEG was performed.
   Compare Normal older (NOLD) participants to AD participants
   Observe the effect of NTRX-07 treatment for 7 days on the qEEG in AD participants
For the current analysis, subjects are pooled in several groups:
   Normal older (NOLD) participants in placebo condition (N=6),
   Alzheimer patients (N=8)
For some analysis, the Alzheimer patient group is split into:
   Alzheimer patients receiving NTRX-07 (N=6),
   Alzheimer patients in placebo condition (N=2)

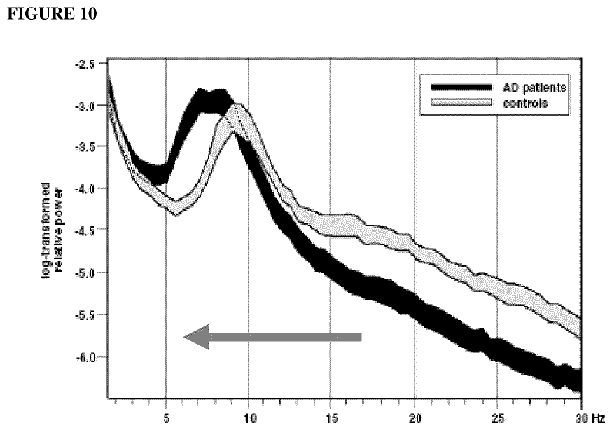

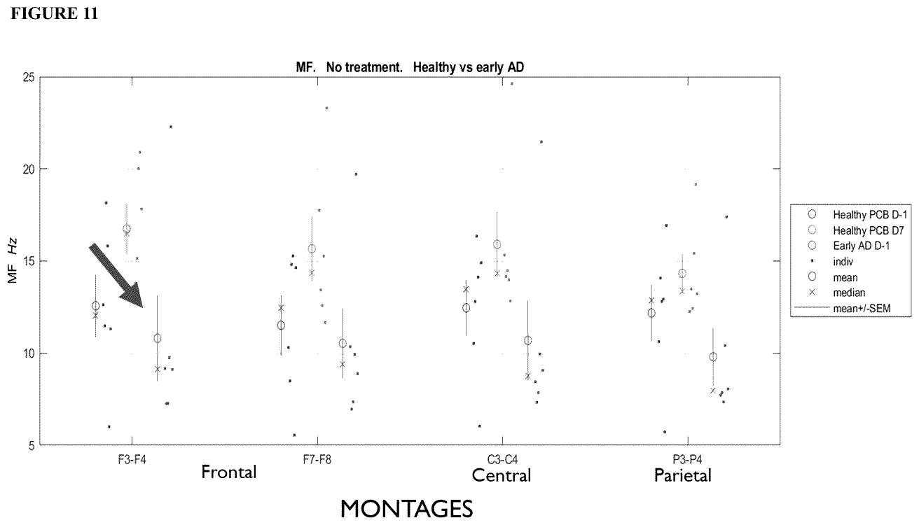

AD is expected to cause a drop in mean frequency (FIG. 10). Clinical Neurophysiology (2007) 118:186-196. The expected drop was observed (FIG. 11). NTRX-07 demonstrated a strong drug effect, increasing mean frequency (FIG. 9).

There were also no dose limiting toxicities, and no SAEs or TEAEs presented for 90 mg cohorts (orthostasis not included) (Table 2). Plasma NTRX-07 levels were consistent with levels predicted to be efficacious based on animal modeling. A summary of PK parameters (mean) is shown in Table 4.

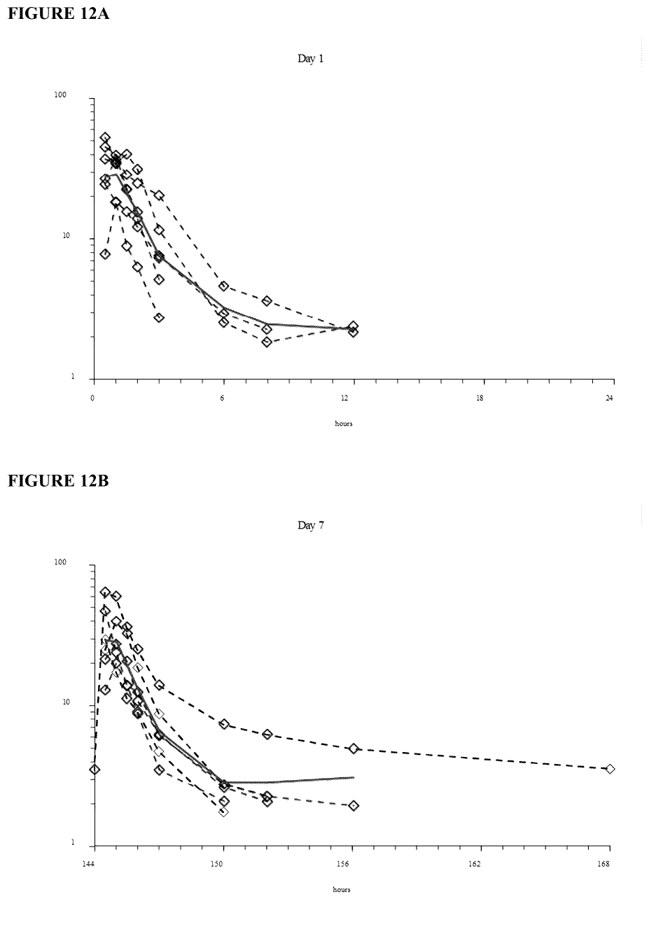

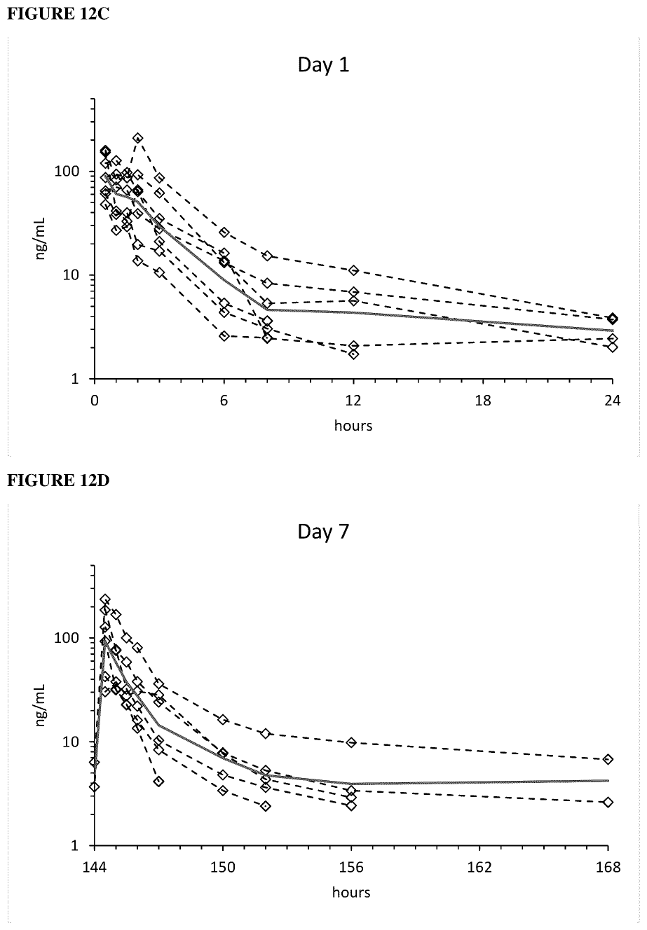

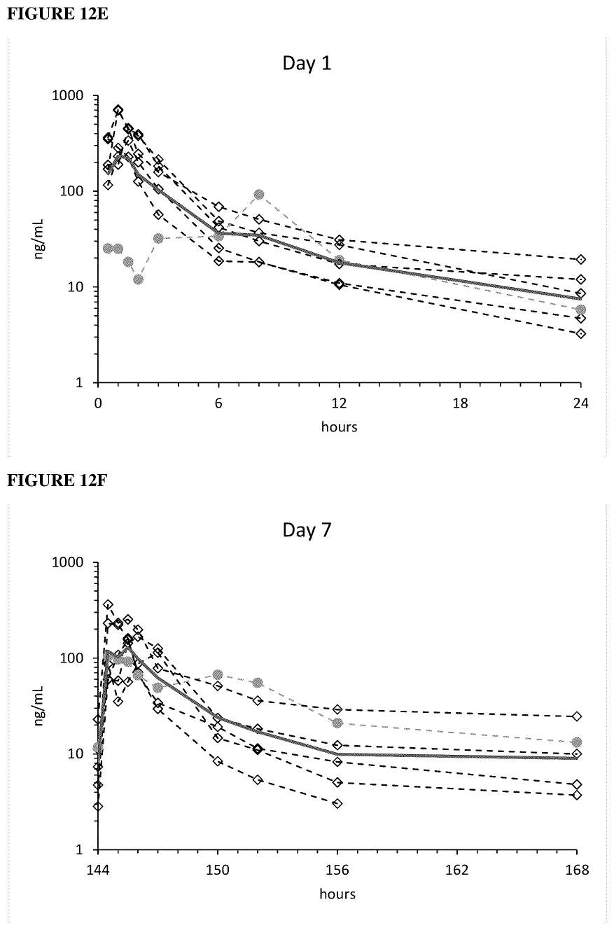

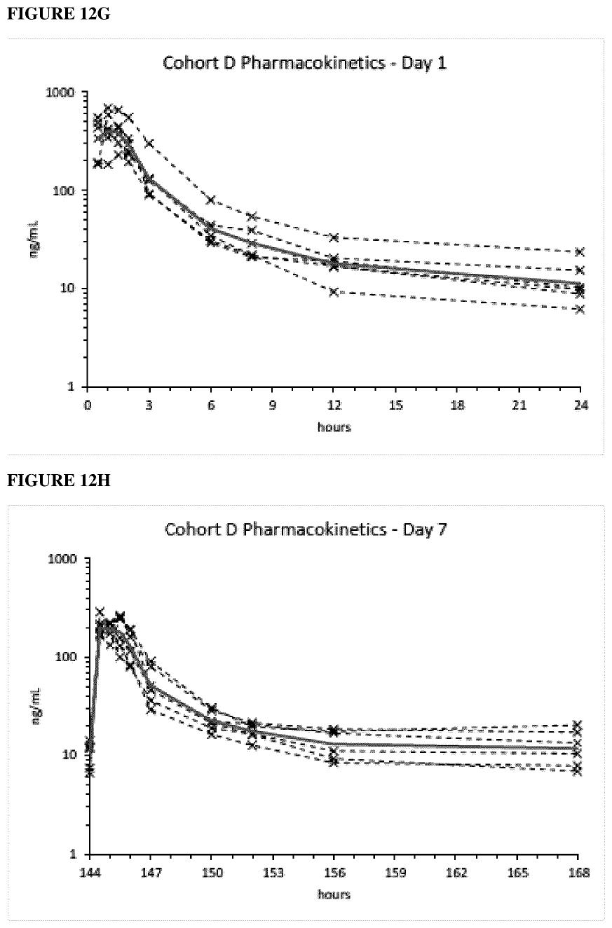

Plasma NTRX-07 PK data are shown in FIGS. 8A-8B, with individual subject data by dose shown in FIGS. 12A-12H.

### Plasma Pharmacokinetic Summary

PK appears to be linear over the dose range studied. Plasma levels were comparable between the volunteers and the AD group. The data generally fit well to a 2-compartment PK model. Day 7 data suggests that clearance may shift slightly at the higher doses. A food effect was observed with muting of Cmax and extension of plasma levels but little or no change in AUC in the fed state.

### Exploratory Endpoints

Exploratory endpoints include cognitive testing and potential plasma biomarkers of inflammation. The small group size (6 on treatment, 2 placebo) precludes statistical analysis, but the goals were to gain insights for future study design. Exploratory endpoints include:
MMSE, MOCA & ADAS-COG
IL-12p70, IL-10, IL-4, IL-5, INFg, IL-6, IL-8, IL-22, TNFa, IL-10, hCRP, Chitinase-3, TREM-2, IL-2, Neurogranin, Total Tau, AB42, AB40, NF-LIGHT, pTau-181

No appreciable changes were seen in MMSE or MOCA; individual subject data by dose are shown in Table 7 below:

TABLE 7
Cognitive Testing: MOCA Exploratory Endpoint

| | | | EOS: |
|---|---|---|---|
| | Day −2 | Day 8 | (Day 14-19) |
| Placebo | | | |
| 042 | 15 | 22 | 19 |
| 050 | 14 | 15 | 13 |
| NTRX-07 | | | |
| 046 | 22 | 22 | 25 |
| 048 | 21 | 21 | 15 |
| 051 | 21 | 19 | 20 |
| 052 | 24 | 20 | 23 |
| 056 | 18 | 19 | 22 |
| 058 | 17 | 17 | 19 |
| Mean NTRX-07 (StdDev) | 20.5 ± 2.6 | 19.7 ± 1.8 | 20.7 ± 3.5 |
| Mean Placebo (StdDev) | 14.5 ± 0.7 | 18.5 ± 4.9 | 16.0 ± 4.2 |

For ADAS-COG, there was a trend toward improvement in all treated NTRX-07 subjects immediately post-treatment:

| | | | EOS: |
|---|---|---|---|
| ADAS-COG | Day −2 | Day 8 | Day 14-19 |
| NTRX-07 | | | |
| 046 | 17.00 | 11.00 | 9.33 |
| 048 | 18.33 | 15.33 | 19.33 |
| 051 | 13.33 | 11.67 | 11.00 |
| 052 | 22.00 | 12.67 | 12.00 |
| 056 | 18.00 | 12.00 | 13.00 |
| 058 | 16.33 | 13.67 | 14.33 |
| Placebo | | | |
| 042 | 13.00 | 13.00 | 16.33 |
| 050 | 12.67 | 16.00 | 17.33 |
| Mean NTRX-07 (StdDev) | 17.50 (2.83) | 12.72 (1.56) | 13.17 (3.46) |
| Mean Placebo (StdDev) | 12.83 (0.23) | 14.50 (2.12) | 16.83 (0.70) |

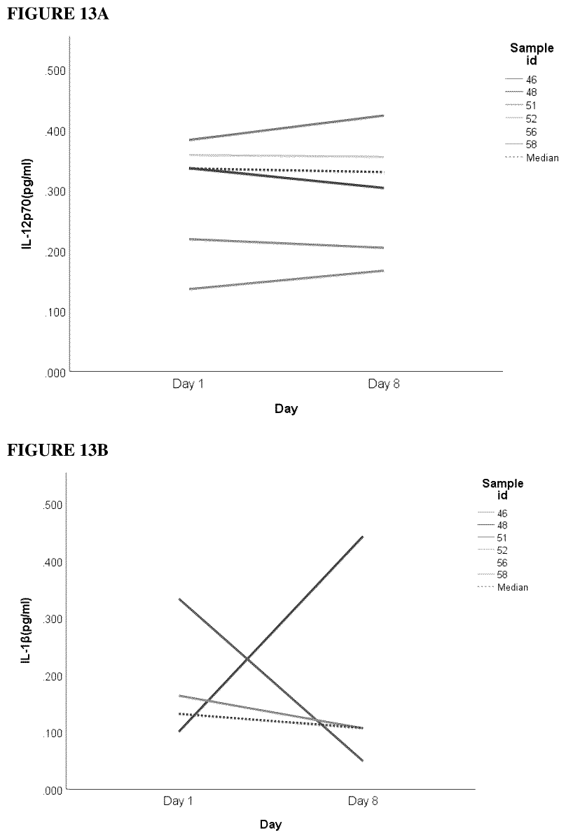

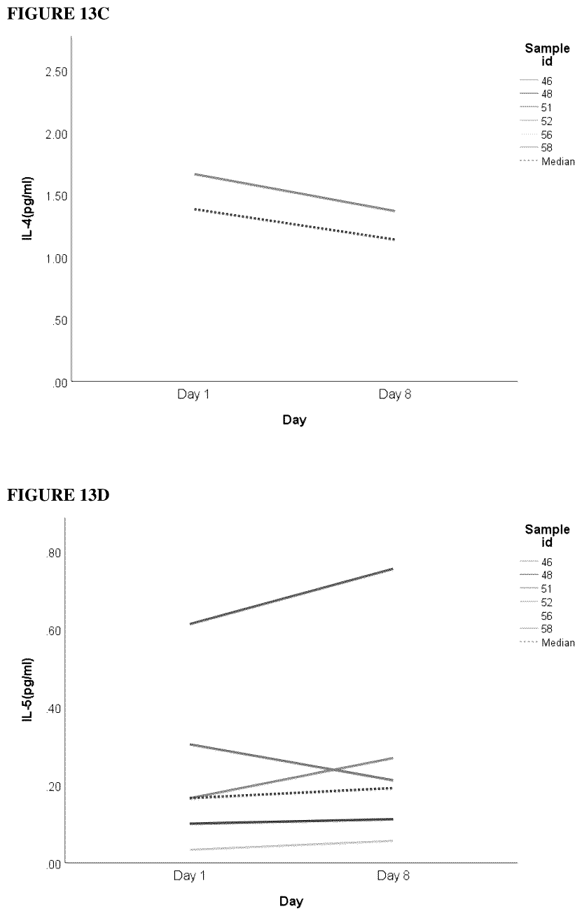

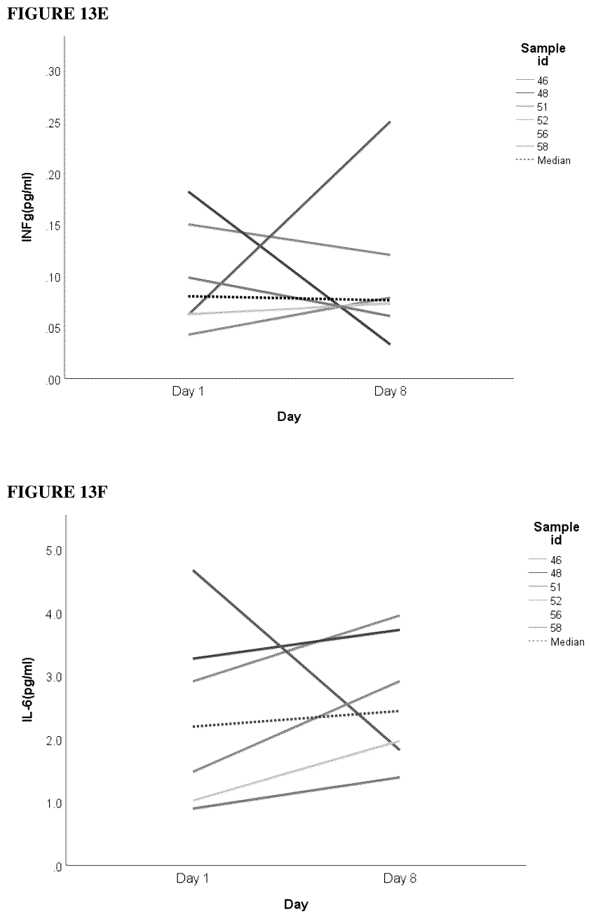

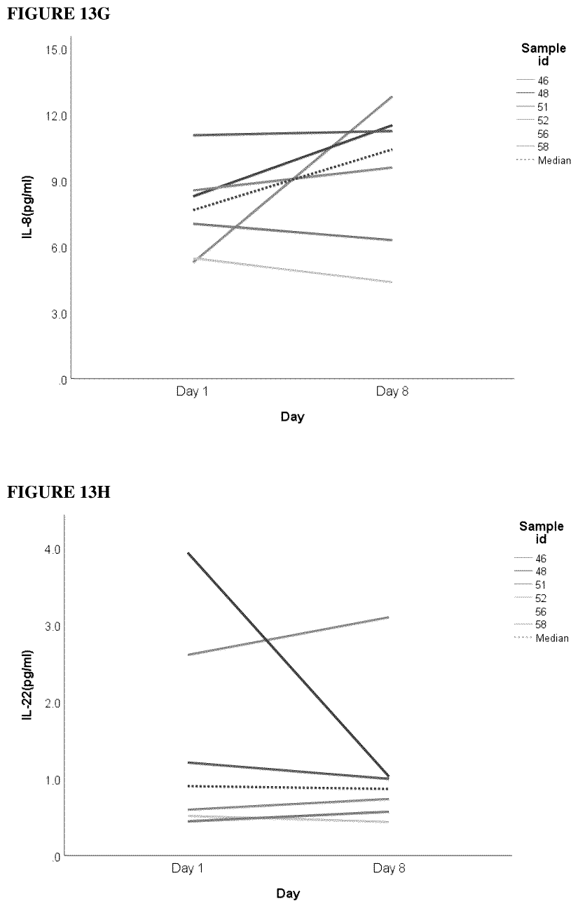

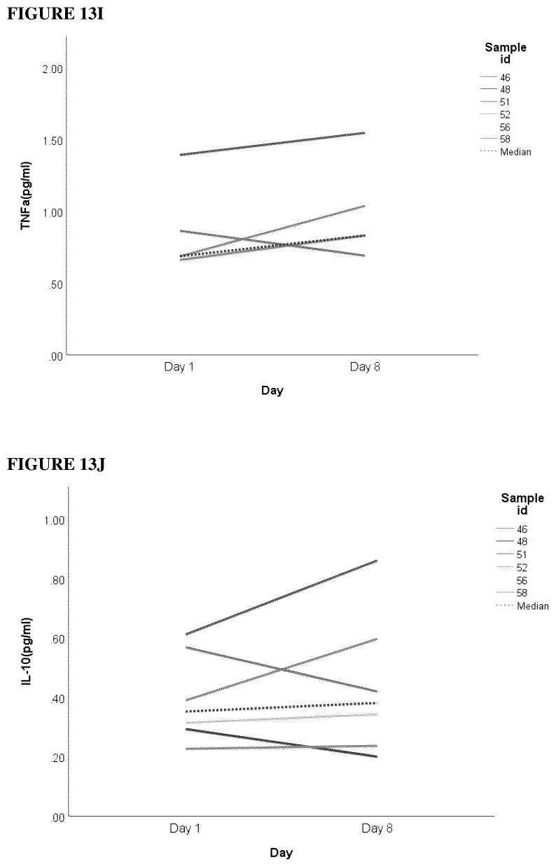

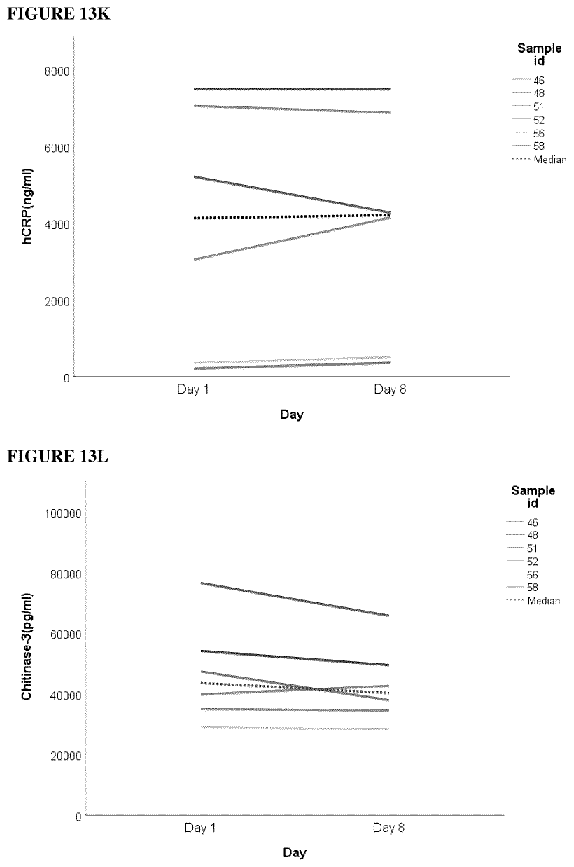

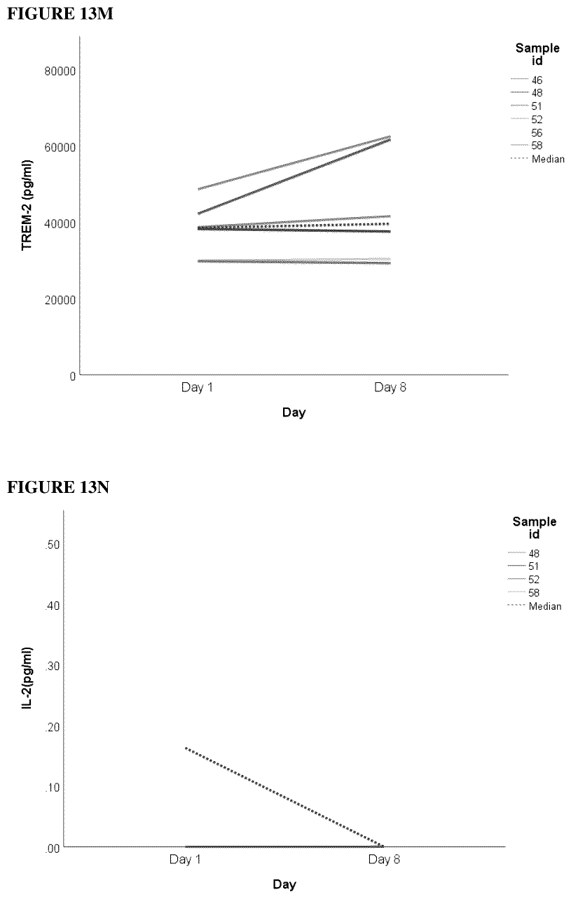

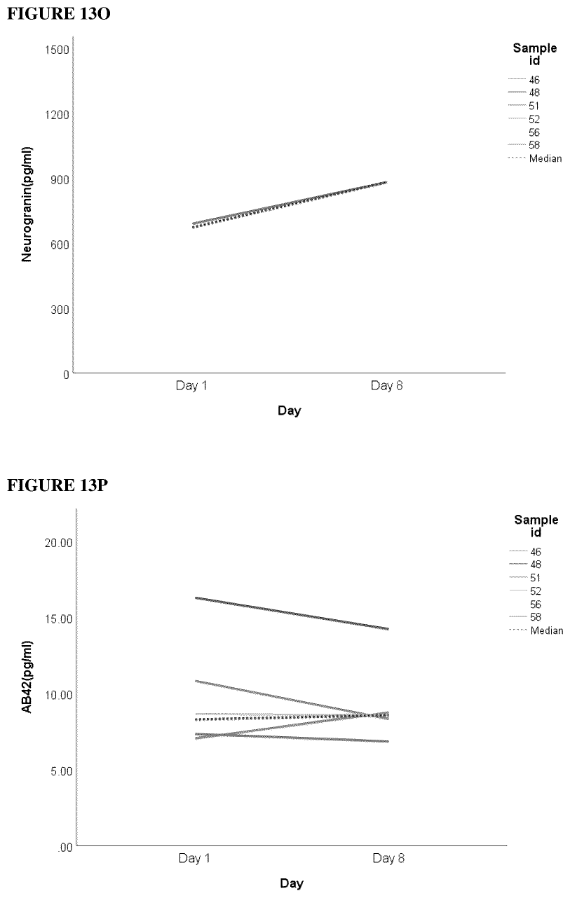

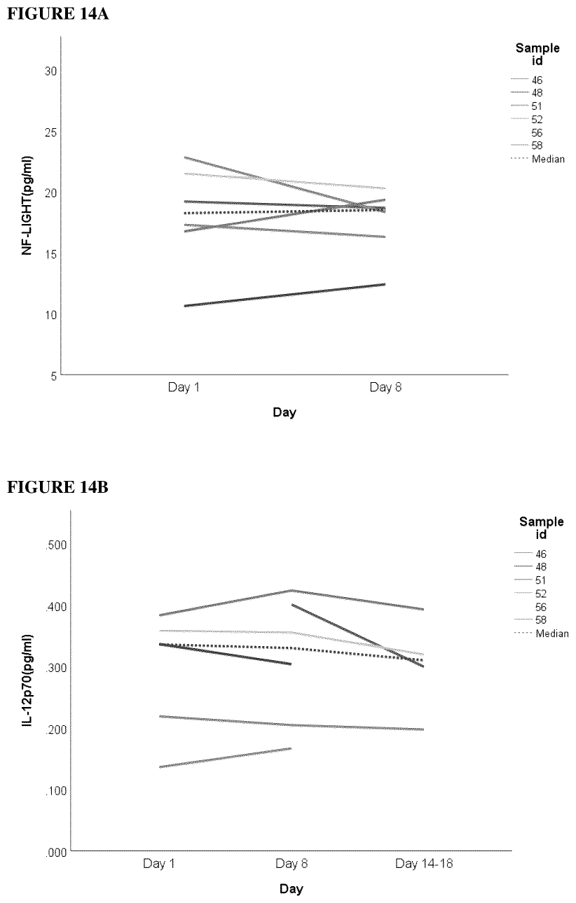

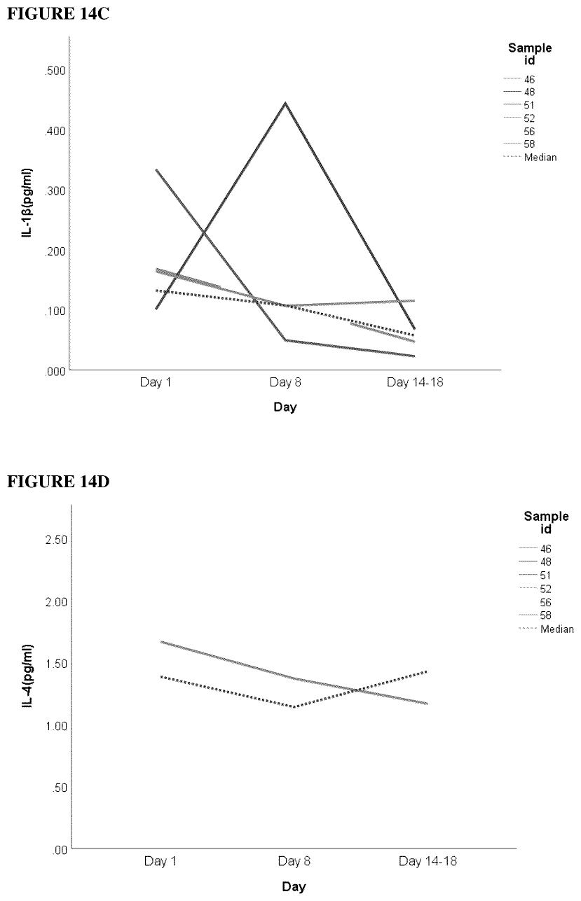

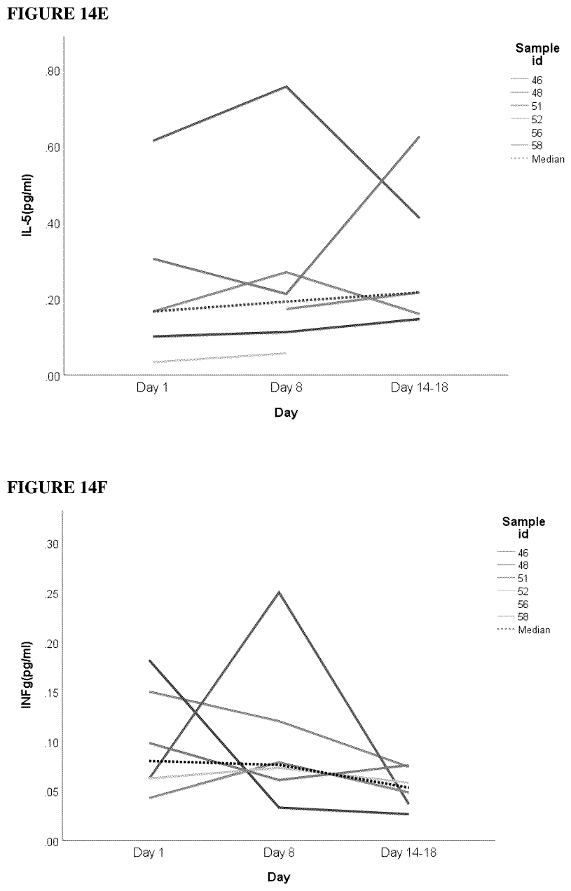

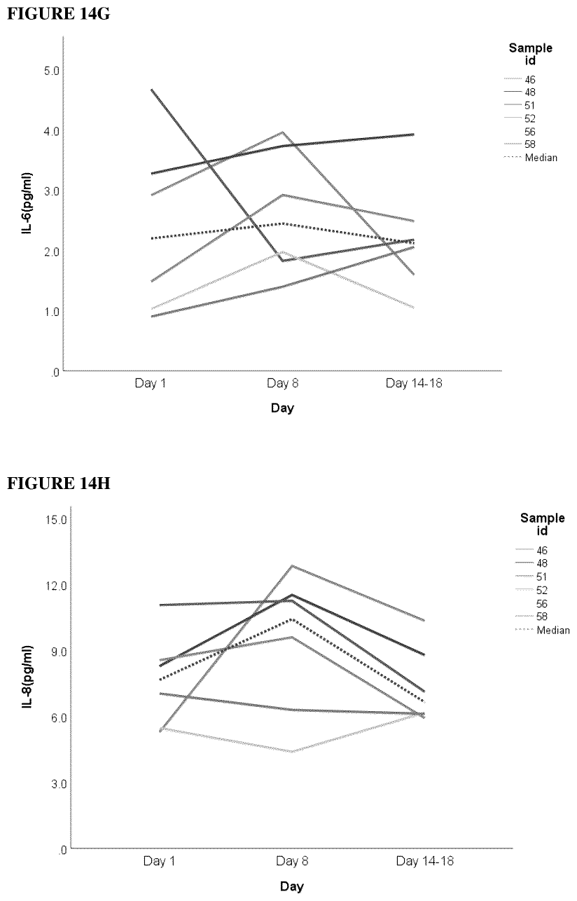

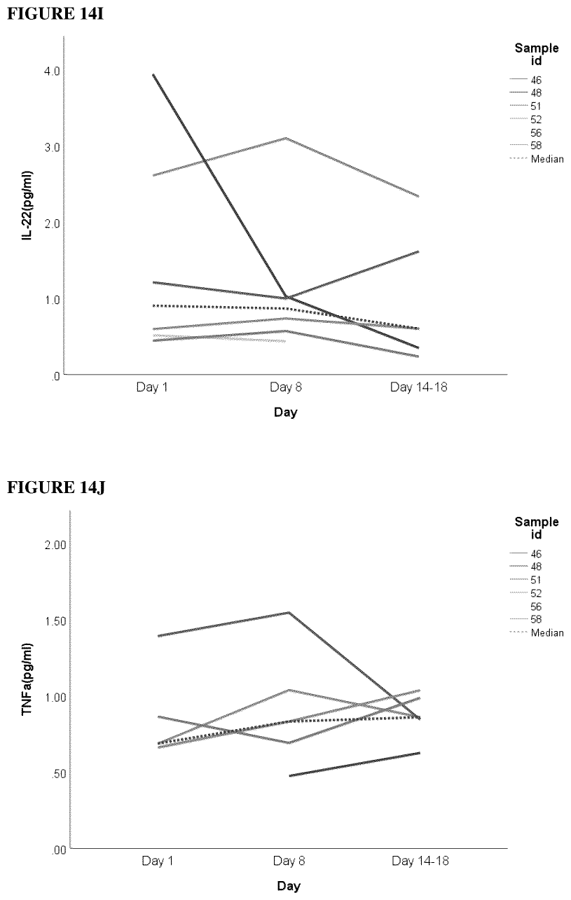

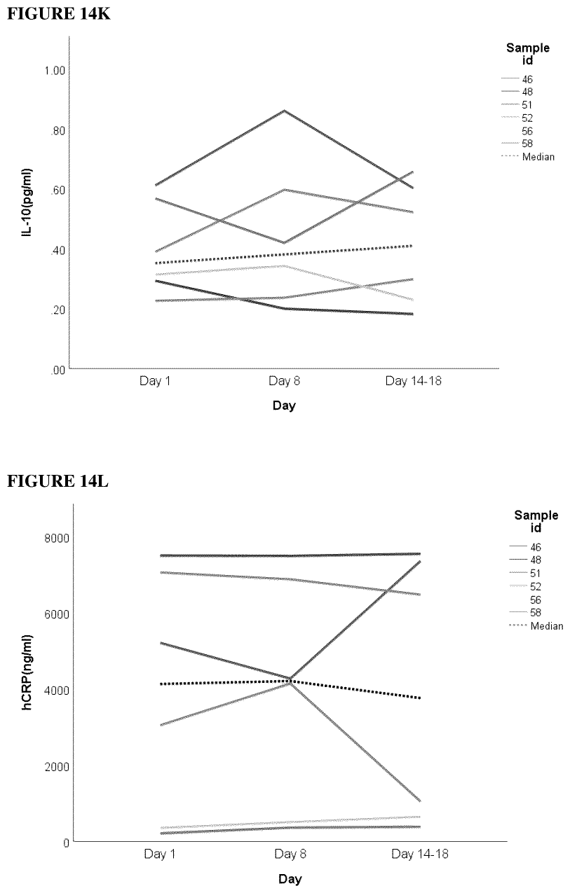

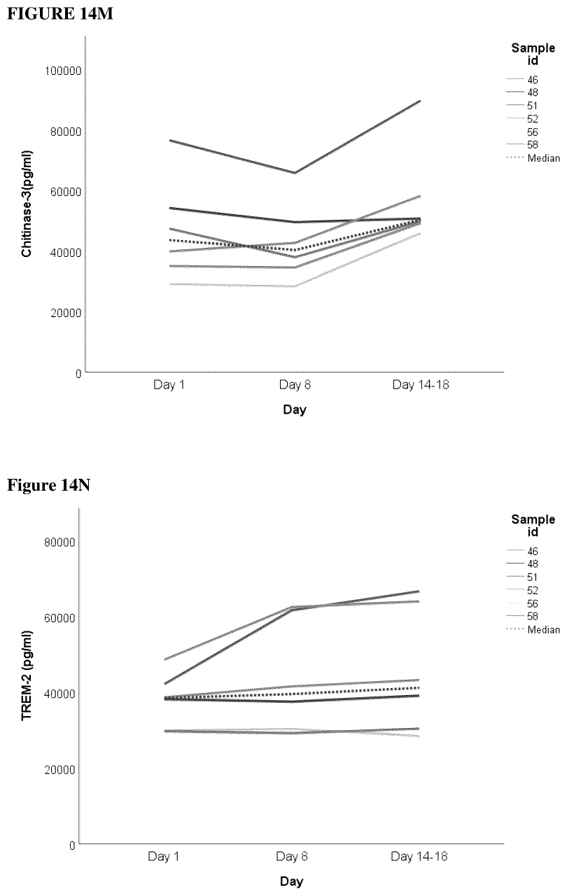

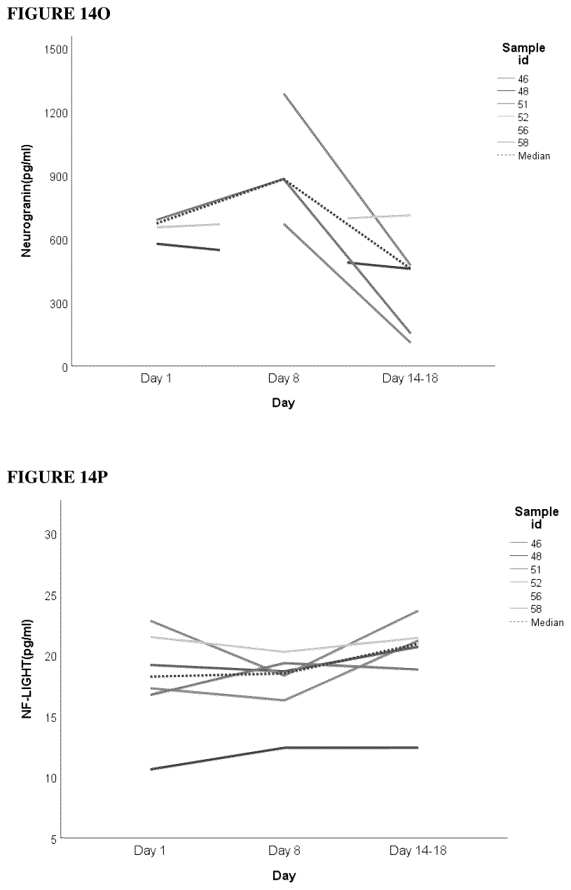

For the inflammatory plasma biomarkers exploratory endpoint, visual inspection of changes in potential inflammatory biomarkers was notable for wide variation in baseline values; Table 5 shows the change from baseline in biomarkers after 7 days of 90 mg NTRX-07 treatment in the AD cohort, FIGS. 13A-13P graphically show changes from baseline in biomarkers after 7 days of 90 mg NTRX-07 treatment in the AD cohort, and FIGS. 14A-14P graphically show changes from baseline to day 14 in biomarkers after 7 days of 90 mg NTRX-07 treatment in the AD cohort. Individual subjects had changes from baseline in individual biomarkers, with some increasing and some decreasing, but there was no consistent changes across any set of biomarkers studied.

### Phase 1B Multiple Ascending Dose Summary

No evidence of vertigo based on three different tests for vertigo
No evidence of abnormal brain activity
6 incidences of orthostasis, no real difference in treatment vs placebo
   Incidences
      16.7% in treated subjects
      25.0% in placebo subjects
   Subjects who had an incident
      16.7% in treated subjects
      12.5% in placebo subjects
Plasma drug exposure at 90 mg dose consistent with predicted efficacious dose
Food effects Cₘₐₓ but has no effect on AUC
Trend toward improvement in ADAS-COG
Expected drop in qEEG mean frequency observed in AD cohort; partially reversed by NTRX-07 treatment

### Phase 2A Study

A study protocol will be reviewed in Europe. Safety will not continue to be monitored by 24 hour EEG. Human bioequivalence testing will not be conducted if in vitro dissolution between powder and tablets is similar.

### Example 4: Summary of NTRX-07 PK Data, Cohorts A Through D

### 1. Background

This report provides a review and analysis of pharmacokinetic (PK) data generated from subjects dosed with 10, 30, or 90 mg NTRX-07 once daily for seven days in a multiple ascending dose (MAD) clinical trial.

### 2. Methodology

Raw bioanalytical data were received from Nuvisan following analysis of plasma samples from each of four cohorts. PK parameters were calculated by noncompartmental analysis using standard equations. Specifically, area under the plasma concentration versus time curve (AUC) was calculated using the trapezoid rule. Maximal plasma concentration (Cmax) and time of maximal plasma concentration (Tmax) were taken directly from the data. Clearance divided by bioavailability (Cl/F) was calculated as the dose divided by the AUC. Apparent volume of distribution over bioavailability (Vd/F) was calculated as dose divided by Cmax.

### 3. Summary of the PK Data

With oral administration of the dose every 24 hours for 7 days, blood samples were taken over a 24-hour interval on Days 1 and 7 and before and after dosing on Day 3. Specifically, sampling was performed as shown in Table 8:

TABLE 8
PK sampling scheme

| Study Day | Day 1 | Day 3 | Day 7 |
|---|---|---|---|
| Sampling Times (hr) | 0, 0.5, 1, 1.5, 2, | −0.5, 1.5 | 0, 0.5, 1, 1.5, 2, |
| | 3, 6, 8, 12, 24 | | 3, 6, 8, 12, 24 |

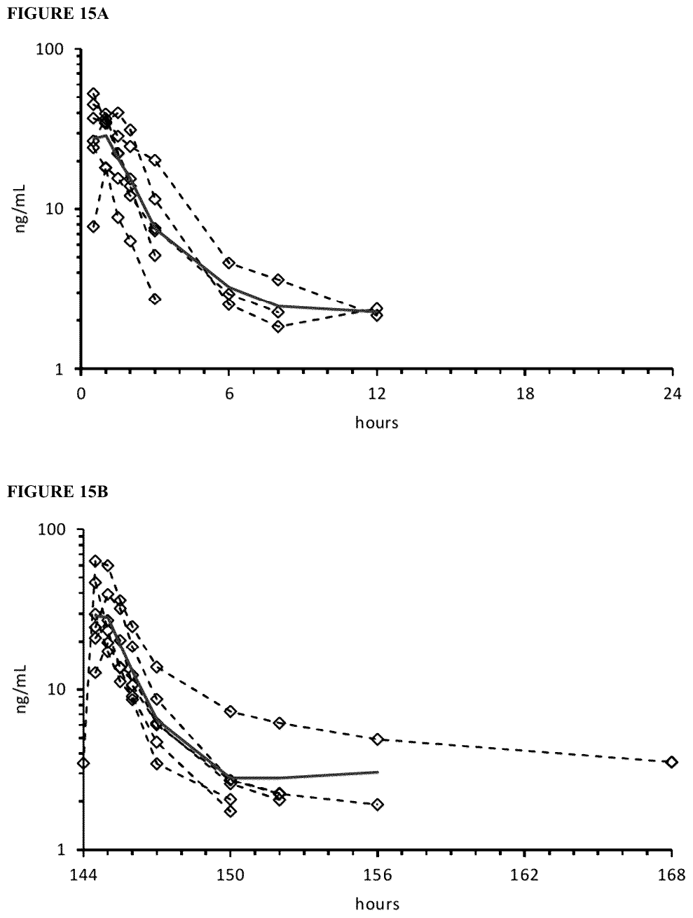

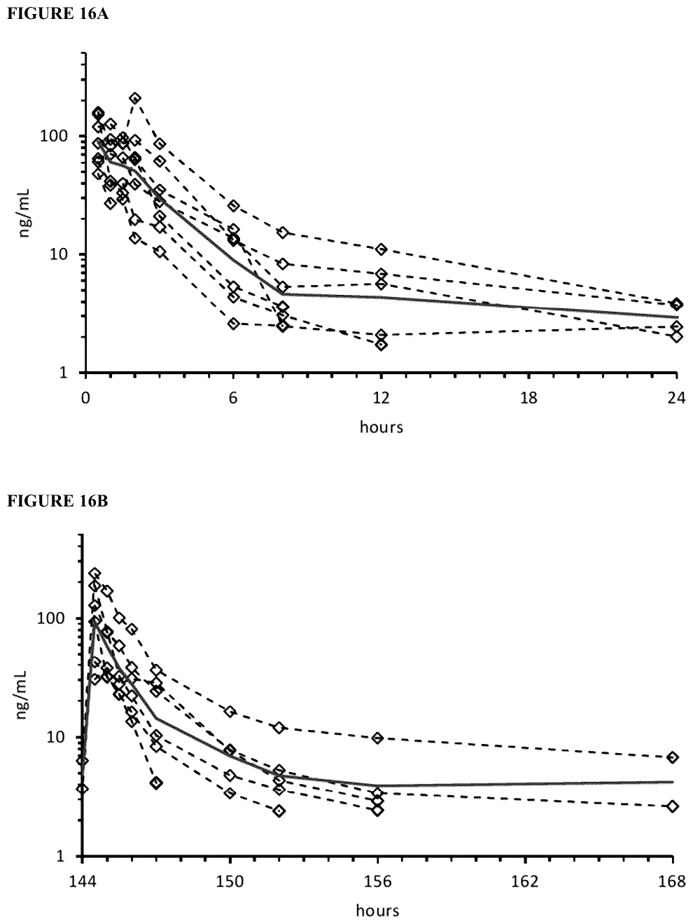

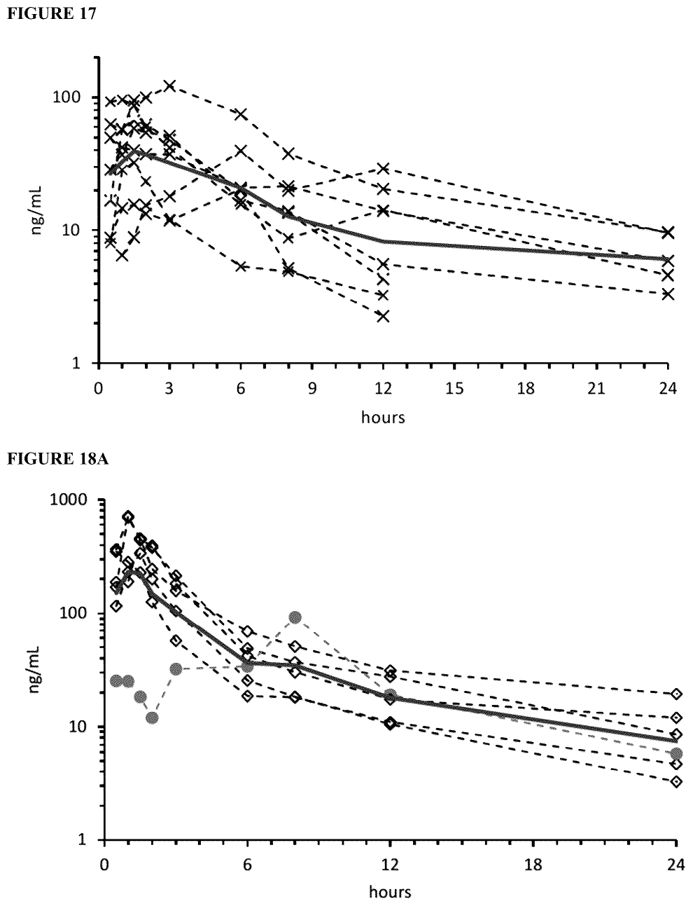

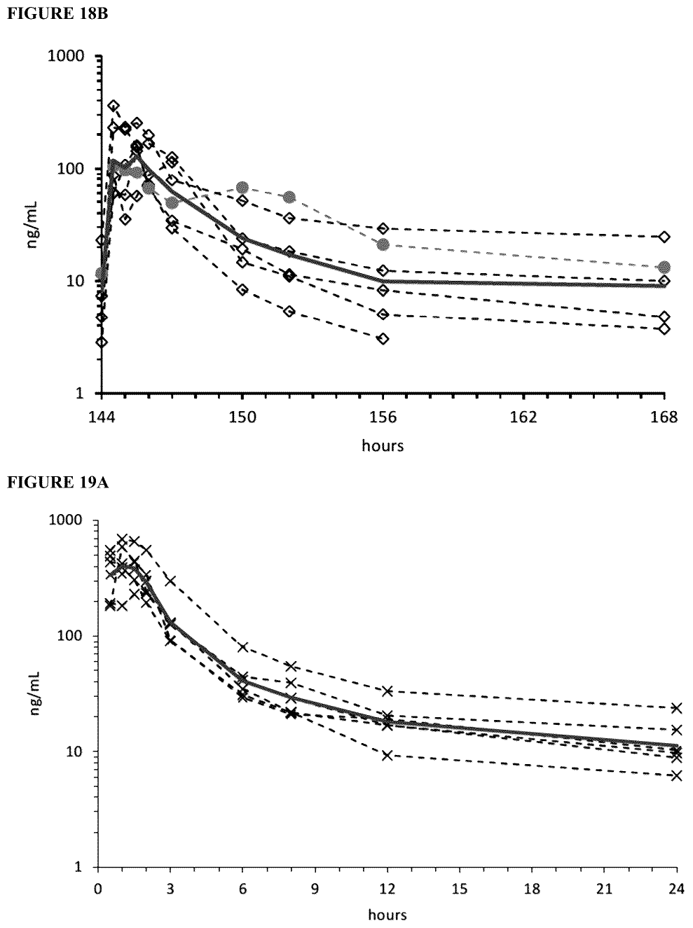

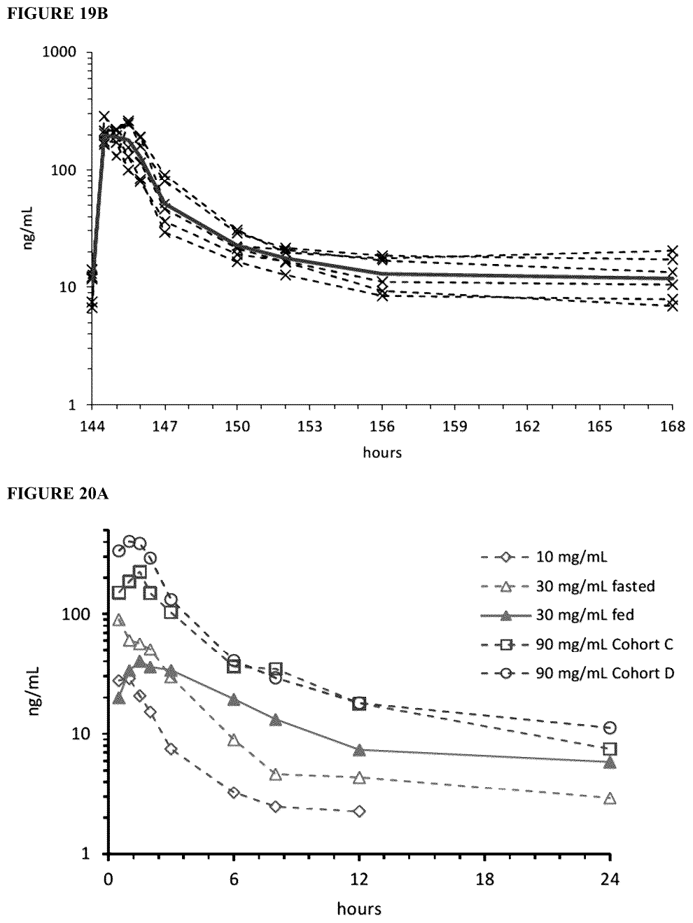

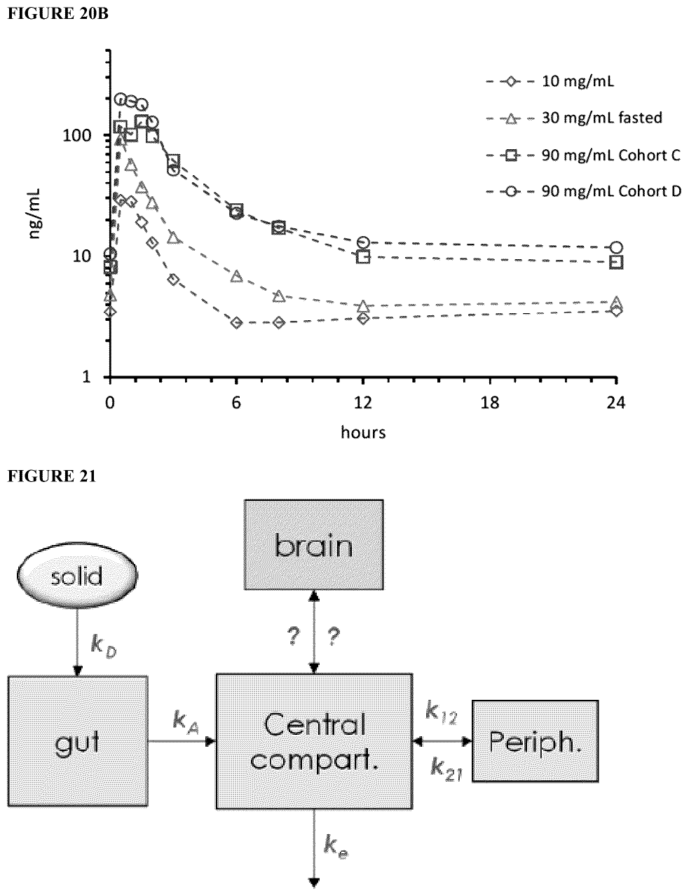

PK data for the subjects in the Cohort A (10 mg dose), Cohort B (30 mg dose), Cohort C (90 mg dose), and Cohort D (90 mg dose) on Day 1 and Day 7 are shown in FIGS. 15A-19B. The red lines on the graphs represent the geometric means of the data. Geometric mean data from the cohorts, including Cohort B fasted and fed, are compared in FIGS. 20A-B.

### 4. Non-Compartmental Analysis

Parameter values obtained from non-compartmental analysis of the PK data are shown in Tables 9-17.

TABLE 9
Cohort A (10 mg QD) Day 1 PK parameters derived from non-compartmental analysis

| Parameter | subj 001 | subj 002 | subj 008 | subj 009 | subj 010 | subj 012 | mean ± S.D. |
|---|---|---|---|---|---|---|---|
| AUCₗₐₛₜ (h · ng/mL) | 31.6 | 66.2 | 131.4 | 33.4 | 126.9 | 83.1 | 78.8 ± 43.7 |
| Cₘₐₓ (ng/mL) | 24.2 | 45.0 | 36.7 | 18.2 | 52.6 | 37.0 | 35.6 ± 12.8 |
| Tₘₐₓ (h) | 0.5 | 0.5 | 1.0 | 1.0 | 0.5 | 0.5 | 0.7 ± 0.3 |
| Cl/F (L/h) | 316.3 | 151.1 | 76.1 | 299.0 | 78.8 | 120.4 | 174 ± 108 |
| V_{d}/F (L) | 1351 | 1015 | 629 | 2041 | 857 | 1476 | 1279 ± 544 |

TABLE 10
Cohort A (10 mg QD) Day 7 PK parameters derived from non-compartmental analysis

| Parameter | subj 001 | subj 002 | subj 008 | subj 009 | subj 010 | subj 012 | mean ± S.D. |
|---|---|---|---|---|---|---|---|
| AUC_{24 h} (h · ng/mL) | 47.8 | 88.4 | 223.8 | 39.5 | 80.4 | 62.9 | 90.4 ± 67.9 |
| Cₘₐₓ (ng/mL) | 29.6 | 39.4 | 63.6 | 19.6 | 46.7 | 27.1 | 37.7 ± 15.9 |
| Tₘₐₓ (h) | 0.5 | 1.0 | 0.5 | 1.0 | 0.5 | 1.0 | 0.8 ± 0.3 |
| Cl/F (L/h) | 209.3 | 113.2 | 44.7 | 253.4 | 124.4 | 158.9 | 151 ± 74 |
| V_{d}/F (L) | 338 | 254 | 157 | 510 | 214 | 369 | 307 ± 126 |

TABLE 11
Cohort B (30 mg QD) Day 1 PK parameters derived from non-compartmental analysis

| Parameter | subj 014 | subj 015 | subj 018 | subj 020 | subj 023 | subj 024 | subj 025 | mean ± S.D. |
|---|---|---|---|---|---|---|---|---|
| AUCₗₐₛₜ (h · ng/mL) | 141.9 | 462.4 | 716.1 | 369.3 | 222.6 | 92.5 | 293.8 | 328 ± 213 |
| Cₘₐₓ (ng/mL) | 60.8 | 93.9 | 209 | 127 | 153 | 47.9 | 70.6 | 109 ± 58 |
| Tₘₐₓ (h) | 0.5 | 1.0 | 2.0 | 1.0 | 0.5 | 0.5 | 1.0 | 0.9 ± 0.5 |
| Cl/F (L/h) | 211.4 | 64.9 | 41.9 | 81.2 | 134.7 | 324.4 | 102.1 | 137 ± 99 |
| V_{d}/F (L) | 493 | 319 | 144 | 236 | 196 | 626 | 425 | 349 ± 175 |

TABLE 12
Cohort B (30 mg QD) Day 7 PK parameters derived from non-compartmental analysis

| Parameter | subj 014 | subj 015 | subj 018 | subj 020 | subj 023 | subj 024 | subj 025 | mean ± S.D. |
|---|---|---|---|---|---|---|---|---|
| AUCₗₐₛₜ (h · ng/mL) | 113 | no data | 581 | 286 | 212 | 61 | 166 | 236 ± 186 |
| Cₘₐₓ (ng/mL) | 92.9 | | 236 | 127 | 186 | 42.4 | 37.9 | 120 ± 79 |
| Tₘₐₓ (h) | 0.5 | | 0.5 | 0.5 | 0.5 | 0.5 | 1 | 0.6 ± 0.2 |
| Cl/F (L/h) | 265.0 | | 51.7 | 105.0 | 141.2 | 492.7 | 181.3 | 206 ± 176 |
| V_{d}/F (L) | 323 | | 127 | 236 | 161 | 708 | 792 | 391 ± 234 |

TABLE 13
Cohort B Fed (30 mg fed state) PK parameters derived from non-compartmental
analysis

| Parameter | sbj 014 | sbj 018 | sbj 020 | sbj 021 | sbj 022 | sbj 023 | sbj 24 | sbj 25 | mean ± S.D. |
|---|---|---|---|---|---|---|---|---|---|
| AUCₗₐₛₜ (h · ng/mL) | 421 | 944 | 471 | 347 | 110 | 312 | 124 | 364 | 390 ± 280 |
| Cₘₐₓ (ng/mL) | 42 | 12 | 95.5 | 39.6 | 32.3 | 66.8 | 20.3 | 58.4 | 60 ± 37 |
| Tₘₐₓ (h) | 1 | 3 | 1 | 3 | 1.5 | 1.5 | 6 | 1.5 | 2.4 ± 1.8 |
| Cl/F (L/h) | 71.2 | 31.8 | 63.7 | 86.4 | 272.1 | 96.3 | 242.3 | 82.5 | 123 ± 94 |
| V_{d}/F (L) | 714 | 248 | 314 | 758 | 929 | 449 | 1478 | 514 | 699 ± 424 |

TABLE 14
Cohort C (90 mg QD) Day 1 PK parameters derived from non-compartmental analysis

| Parameter | subj 026 | subj 027 | subj 028 | subj 034 | subj 039 | subj 041 | mean ± S.D. |
|---|---|---|---|---|---|---|---|
| AUC_{24 h} (h · ng/mL) | 1629 | 857 | 925 | 657 | 1730 | 1807 | 1267 ± 509 |
| Cₘₐₓ (ng/mL) | 449 | 350 | 337 | 92.4 | 713 | 456 | 400 ± 202 |
| Tₘₐₓ (h) | 1.5 | 0.5 | 1.5 | 8 | 1 | 1.5 | 2.3 ± 2.2 |
| Cl/F (L/h) | 55.2 | 105.1 | 97.3 | 137.0 | 52.0 | 49.8 | 83 ± 36 |
| V_{d}/F (L) | 200 | 257 | 267 | 974 | 126 | 197 | 337 ± 316 |

TABLE 15
Cohort C (90 mg QD) Day 7 PK parameters derived from non-compartmental analysis

| Parameter | subj 026 | subj 027 | subj 028 | subj 034 | subj 039 | subj 041 | mean ± S.D. |
|---|---|---|---|---|---|---|---|
| AUC_{24 h} (h · ng/mL) | 783 | 604 | 318 | 516 | 876 | 1349 | 741 ± 357 |
| Cₘₐₓ (ng/mL) | 167 | 230 | 155 | 114 | 105 | 361 | 189 ± 95 |
| Tₘₐₓ (h) | 2 | 0.5 | 1.5 | 3 | 0.5 | 0.5 | 1.3 ± 1.0 |
| Cl/F (L/h) | 115.0 | 149.1 | 283.5 | 174.4 | 102.7 | 66.7 | 149 ± 76 |
| V_{d}/F (L) | 539 | 391 | 581 | 789 | 857 | 249 | 568 ± 231 |

TABLE 16
Cohort D (90 mg QD) Day 1 PK parameters derived from non-compartmental analysis

| Parameter | subj 046 | subj 048 | subj 051 | subj 052 | subj 056 | subj 058 | mean ± S.D. |
|---|---|---|---|---|---|---|---|
| AUCₗₐₛₜ (h · ng/mL) | 1004 | 1255 | 2727 | 1349 | 1580 | 1422 | 1556 ± 605 |
| Cₘₐₓ (ng/mL) | 234 | 491 | 685 | 435 | 434 | 587 | 478 ± 154 |
| Tₘₐₓ (h) | 2 | 0.5 | 1 | 1.5 | 0.5 | 1 | 1.1 ± 0.6 |
| Cl/F (L/h) | 89.6 | 71.7 | 33.0 | 66.7 | 57.0 | 63.3 | 64 ± 19 |
| V_{d}/F (L) | 385 | 183 | 131 | 207 | 207 | 153 | 211 ± 90 |

TABLE 17
Cohort D (90 mg QD) Day 7 PK parameters derived from non-compartmental analysis

| Parameter | subj 046 | subj 048 | subj 051 | subj 052 | subj 056 | subj 058 | mean ± S.D. |
|---|---|---|---|---|---|---|---|
| AUC_{24 h} (h · ng/mL) | 623 | 1067 | 994 | 856 | 608 | 689 | 806 ± 196 |
| Cₘₐₓ (ng/mL) | 286 | 250 | 262 | 246 | 176 | 221 | 240 ± 38 |
| Tₘₐₓ (h) | 0.5 | 1.5 | 1.5 | 1.5 | 0.5 | 1 | 1.1 ± 0.5 |
| Cl/F (L/h) | 144.6 | 84.4 | 90.5 | 105.2 | 148.0 | 130.6 | 117 ± 28 |
| V_{d}/F (L) | 315 | 360 | 344 | 366 | 511 | 407 | 384 ± 69 |

Note that large values for Cl/F and Vd/F can reflect poor bioavailability, broad distribution in the body or a combination of the two.

### 5. Predictive Compartmental Analysis

Predictive compartmental PK analysis was utilized during dose escalation to predict PK outcomes at higher dose levels based on data from lower ones. A two-compartment scheme upon which the mathematical compartmental model is based is shown in FIG. 21.

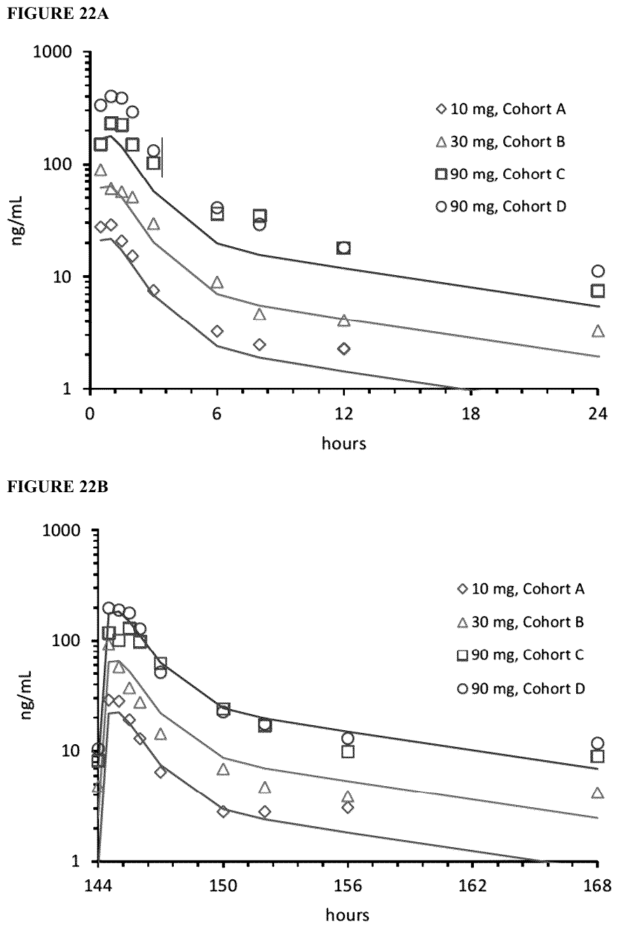

FIGS. 22A and 22B show the mathematical model with a simultaneous fit to data from all four cohorts (excluding fed subject data). Since the model does not take into account factors such as enzyme inhibition, enzyme induction, and other saturable clearance or absorption mechanisms, agreement between data and the model is indicative of these factors not being important to the PK. The generally adequate fit across doses suggests that PK is linear across this dose range, but the poorer fit for Day 1 than for Day 7 for the high dose cohorts suggests the possibility that clearance might shift slightly over time with repeated dosing.

### 6. Conclusions

PK appears to be linear over the dose range studied.
The data generally fit well to a 2-compartment PK model.
A marked food effect was observed with muting of Cmax and extension of plasma levels but little or no change in AUC in the fed state.

### Example 5: Interim PK Analysis NTRX-07 MAD Cohort A

As used in Example 5, an amount of “NTRX-07” refers to an amount of a spray-dried dispersion (SDD) of NTRX-07, containing 75% SDD and 25% NTRX-07, e.g., “40 mg NTRX-07” refers to 40 mg of an SDD containing 10 mg NTRX-07.

### 1. Background

This report provides an interim review and analysis of pharmacokinetic (PK) data generated from subjects dosed with 40 mg NTRX-07 once daily for seven days as the first cohort of a multiple ascending dose (MAD) clinical trial.

### 2. Methodology

Raw bioanalytical data were received from Nuvisan on Oct. 27, 2022. Parameters derived from noncompartmental analysis of these data were derived using standard equations. Specifically, area under the plasma concentration versus time curve (AUC) was calculated using the trapezoid rule. Maximal plasma concentration (Cmax) and time of maximal plasma concentration (Tmax) were taken directly from the data. Clearance divided by bioavailability (Cl/F) was calculated as the dose divided by the AUC. Apparent volume of distribution over bioavailability (Vd/F) was calculated as dose divided by Cmax.

Compartmental analysis was performed using a two-compartment model with extravascular administration. Modeling was performed using a sophisticated MS Excel workbook developed by PharmaDirections.

### 3. Summary of the PK Data

With oral administration of the dose every 24 hours for 7 days, blood samples were taken over a 24-hour interval on Days 1 and 7 and before and after dosing on Day 3. Specifically, sampling was performed as shown in Table 18.

TABLE 18
PK sampling scheme

| Study Day | Day 1 | Day 3 | Day 7 |
|---|---|---|---|
| Sampling Times (hr) | 0, 0.5, 1, 1.5, 2, | −0.5, 1.5 | 0, 0.5, 1, 1.5, 2, |
| | 3, 6, 8, 12, 24 | | 3, 6, 8, 12, 24 |

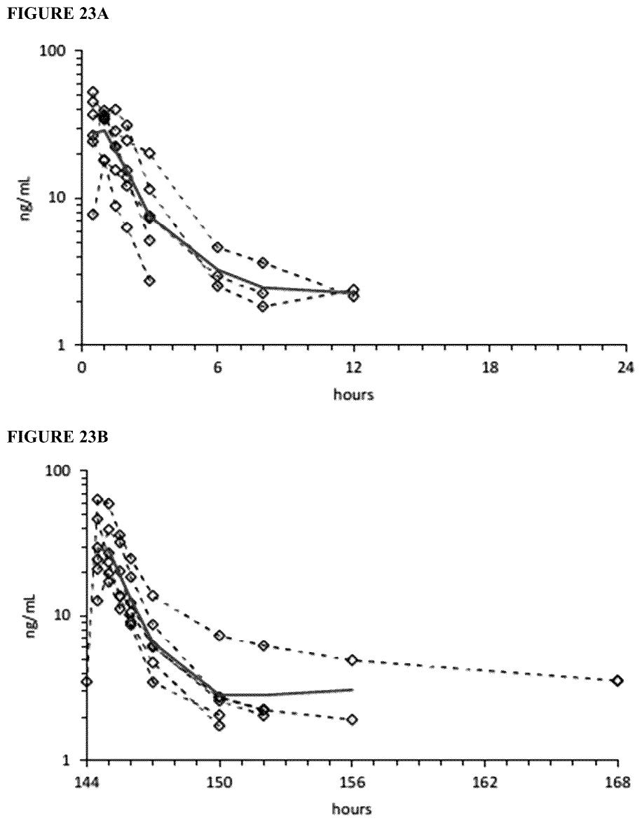

PK data for the six subjects in the cohort on Day 1 and Day 7 are shown in FIGS. 23A-23B.

The red (solid) lines on the graphs represent the geometric means of the data.

### 4. Non-Compartmental Analysis

Parameter values obtained from non-compartmental analysis of the PK data are shown in Tables 19-20.

TABLE 19
Day 1 PK parameters derived from non-compartmental analysis

| Parameter | subj 001 | subj 002 | subj 008 | subj 009 | subj 010 | subj 012 | mean ± S.D. |
|---|---|---|---|---|---|---|---|
| AUC_{24 h} (h · ng/mL) | 35.7 | 73.8 | 151.0 | 46.3 | 154.2 | 96.8 | 93.0 ± 0.9 |
| Cₘₐₓ (ng/mL) | 24.2 | 45.0 | 36.7 | 18.2 | 52.6 | 37.0 | 35.6 ± 12.8 |
| Tₘₐₓ (h) | 0.5 | 0.5 | 1.0 | 1.0 | 0.5 | 0.5 | 0.7 ± 0.3 |
| Cl/F (L/h) | 1120 | 542 | 265 | 864 | 259 | 413 | 577 ± 347 |
| V_{d}/F (L) | 1351 | 1015 | 629 | 2041 | 857 | 1476 | 1279 ± 544 |

TABLE 20
Day 7 PK parameters derived from non-compartmental analysis

| Parameter | subj 001 | subj 002 | subj 008 | subj 009 | subj 010 | subj 012 | mean ± S.D. |
|---|---|---|---|---|---|---|---|
| AUC_{24 h} (h · ng/mL) | 49.5 | 92.8 | 223.8 | 41.5 | 91.9 | 67.0 | 94.4 ± 66.8 |
| Cₘₐₓ (ng/mL) | 29.6 | 39.4 | 63.6 | 19.6 | 46.7 | 27.1 | 37.7 ± 15.9 |
| Tₘₐₓ (h) | 0.5 | 1.0 | 0.5 | 1.0 | 0.5 | 1.0 | 0.8 ± 0.3 |
| Cl/F (L/h) | 808 | 431 | 179 | 963 | 435 | 597 | 569 ± 284 |
| V_{d}/F (L) | 1351 | 1015 | 629 | 2041 | 857 | 1476 | 1228 ± 506 |

Note that large values for Cl/F and Vd/F can reflect poor bioavailability, broad distribution in the body or a combination of the two.

### 5. Predictive Compartmental Analysis

A two-compartment scheme upon which the mathematical compartmental model is based is shown in FIG. 21.

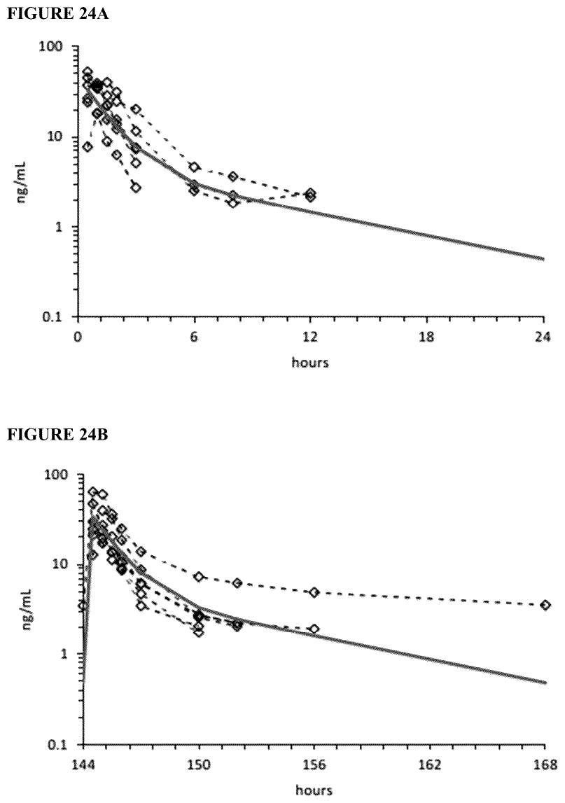

FIGS. 24A-B show the mathematical model (heavy blue line) fit to the Cohort A data. The model will be further refined as data from additional cohorts become available. In particular, the terminal slope may change some, as the current data set is inadequate to define it. The model was built based on geometric mean data, but timepoint data were not used in cases where plasma concentrations were below the limit of quantitation for four or more of the six subjects.

PK parameters derived from the compartmental model are provided in Table 21. Note that the model estimates are somewhat higher than those obtained from non-compartmental analysis since they consider the entire dosing interval as opposed to only the time points for which data are available.

TABLE 21
PK parameters for 40 mg QD dose derived from compartmental PK model

| Parameter | Day 1 | Day 2 | Day 3 | Day 4 | Day 5 | Day 6 | Day 7 | accumulation |
|---|---|---|---|---|---|---|---|---|
| AUC_{24 h} (h · ng/mL) | 96 | 100 | 101 | 101 | 101 | 101 | 101 | 104% |
| Cₘₐₓ (ng/mL) | 43.9 | 44.3 | 44.4 | 44.4 | 44.4 | 44.4 | 44.4 | 101% |
| Cₘᵢₙ (ng/mL) | 0.0 | 0.4 | 0.5 | 0.5 | 0.5 | 0.5 | 0.5 | N/A |
| tₘₐₓ (h) | 0.08 | 0.08 | 0.08 | 0.08 | 0.08 | 0.08 | 0.08 | N/A |

Predicted PK parameters for higher doses of NTRX-07 are shown in Table 22. These predictions are based on an assumption of linear PK across the specified dose range, and parameters could be underestimated if a saturable process limits bioavailability or is involved in drug clearance. Current data are insufficient to determine whether any saturable processes are involved in NTRX-007 uptake or clearance.

TABLE 22
Predicted PK parameters for higher daily doses of NTRX-07

| | QD Dose of NTRX-07 | | | | | | | |
|---|---|---|---|---|---|---|---|---|
| Parameter | 50 mg | 60 mg | 70 mg | 80 mg | 90 mg | 100 mg | 110 mg | 120 mg |
| AUC_{24 h} (h · ng/mL) | 126 | 151 | 176 | 202 | 227 | 252 | 277 | 302 |
| Cₘₐₓ (ng/mL) | 55 | 67 | 78 | 89 | 100 | 111 | 122 | 133 |
| Cₘᵢₙ (ng/mL) | 0.6 | 0.7 | 0.8 | 1.0 | 1.1 | 1.2 | 1.3 | 1.4 |

### 6. Conclusion

Data from Cohort A (40 mg QD) show moderate inter-subject variability and little or no accumulation over the course of seven days of dosing. Predictions for higher doses were provided based on output from a compartmental PK model. The compartmental PK model will be updated as additional data become available.

### Example 6: Interim PK Analysis NTRX-07 MAD Cohort B

As used in Example 6, an amount of “NTRX-07” refers to an amount of a spray-dried dispersion (SDD) of NTRX-07, containing 75% SDD and 25% NTRX-07, e.g., “40 mg NTRX-07” refers to 40 mg of an SDD containing 10 mg NTRX-07, “120 mg NTRX-07” refers to 120 mg of an SDD containing 30 mg NTRX-07, and “360 mg NTRX-07” refers to 360 mg of an SDD containing 90 mg NTRX-07.

### 1. Background

This report provides an interim review and analysis of pharmacokinetic (PK) data generated from subjects dosed with 120 mg NTRX-07 once daily for seven days as the second cohort of a multiple ascending dose (MAD) clinical trial.

### 2. Methodology

Raw bioanalytical data were received from Nuvisan on Nov. 24, 2022. Parameters derived from noncompartmental analysis of these data were calculated using standard equations. Specifically, area under the plasma concentration versus time curve (AUC) was calculated using the trapezoid rule. Maximal plasma concentration (Cmax) and time of maximal plasma concentration (Tmax) were taken directly from the data. Clearance divided by bioavailability (Cl/F) was calculated as the dose divided by the AUC. Apparent volume of distribution over bioavailability (Vd/F) was calculated as dose divided by Cmax.

Compartmental analysis was performed based on a two-compartment model with extravascular administration. Modeling was performed using a sophisticated MS Excel workbook developed by PharmaDirections. Geometric mean data from Cohorts A and B were used to determine weighted least squares best fits for the PK parameters.

### 3. Summary of the PK Data

With oral administration of the dose every 24 hours for 7 days, blood samples were taken over a 24-hour interval on Days 1 and 7 and before and after dosing on Day 3. Specifically, sampling was performed as shown in Table 23.

TABLE 23
PK sampling scheme

| Study Day | Day 1 | Day 3 | Day 7 |
|---|---|---|---|
| Sampling Times (hr) | 0, 0.5, 1, 1.5, 2, | −0.5, 1.5 | 0, 0.5, 1, 1.5, 2, |
| | 3, 6, 8, 12, 24 | | 3, 6, 8, 12, 24 |

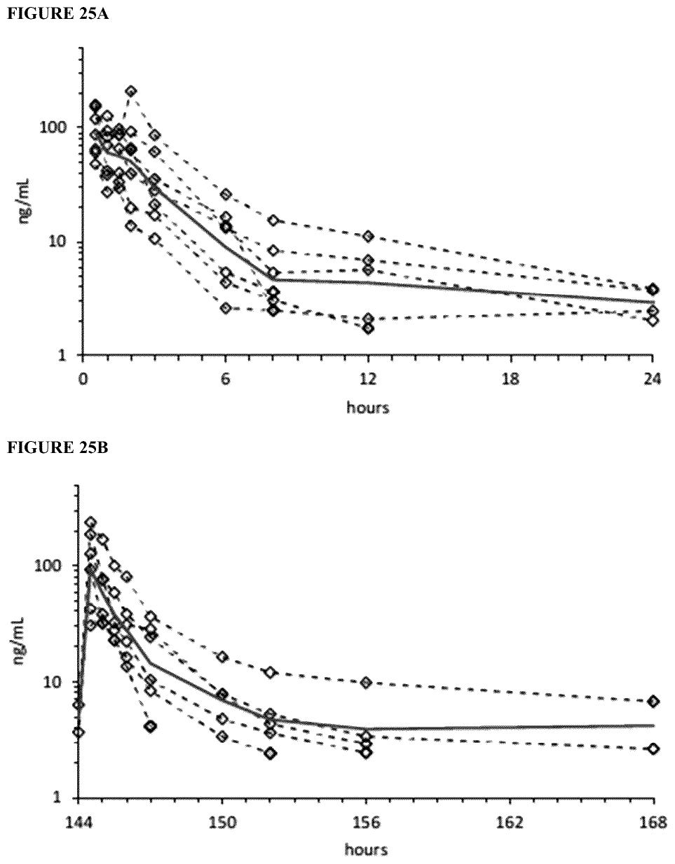

PK data for the seven subjects in the cohort on Day 1 and Day 7 are shown in FIGS. 25A-B. The red lines on the graphs represent the geometric means of the data. Data were missing from one subject for Days 3 and 7, presumably because that subject (Subject #015) dropped out of the study prior to Day 3. Intersubject variability was moderately high.

### 4. Non-Compartmental Analysis

Parameter values obtained from non-compartmental analysis of the PK data are shown in Tables 24-25.

TABLE 24
Day 1 PK parameters derived from non-compartmental analysis

| Parameter | Sbj 014 | Sbj 015 | Sbj 018 | Sbj 020 | Shj 023 | Sbj 024 | Sbj 025 | mean ± S.D. |
|---|---|---|---|---|---|---|---|---|
| AUC_{24 h} (h · ng/mL) | 152.3 | 462.4 | 716.3 | 369.3 | 229.9 | 97.4 | 236.4 | 323.4 ± 213.0 |
| Cₘₐₓ (ng/mL) | 60.8 | 93.9 | 209 | 127 | 153 | 47.9 | 70.6 | 108.9 ± 57.8 |
| Tₘₐₓ (h) | 0.5 | 1 | 2 | 1 | 0.5 | 0.5 | 1 | 0.9 ± 0.6 |
| Cl/F (L/h) | 788 | 260 | 168 | 325 | 522 | 1231 | 508 | 549 ± 401 |
| V_{d}/F (L) | 1974 | 1278 | 574 | 945 | 784 | 2505 | 1700 | 1343 ± 750 |

TABLE 25
Day 7 PK parameters derived from non-compartmental analysis

| Parameter | Sbj 014 | Sbj 015 | Sbj 018 | Sbj 020 | Sbj 023 | Sbj 024 | Sbj 025 | mean ± S.D. |
|---|---|---|---|---|---|---|---|---|
| AUC_{24 h} (h · ng/mL) | 118.0 | No data | 582.3 | 286.5 | 241.7 | 67.1 | 200.4 | 213.7 ± 190.8 |
| Cₘₐₓ (ng/mL) | 92.9 | available | 236 | 127 | 186 | 42.4 | 37.9 | 120.4 ± 79.2 |
| Tₘₐₓ (h) | 0.5 | | 0.5 | 0.5 | 0.5 | 0.5 | 1 | 0.5 ± 0.0 |
| Cl/F (L/h) | 1017 | | 206 | 419 | 497 | 1788 | 599 | 785 ± 635 |
| V_{d}/F (L) | 1292 | | 508 | 945 | 645 | 2830 | 3166 | 1244 ± 936 |

Note that large values for Cl/F and Vd/F can reflect poor bioavailability, broad distribution in the body or a combination of the two.

### 5. Predictive Compartmental Analysis

A two-compartment scheme upon which the mathematical compartmental model is based is shown in FIG. 21.

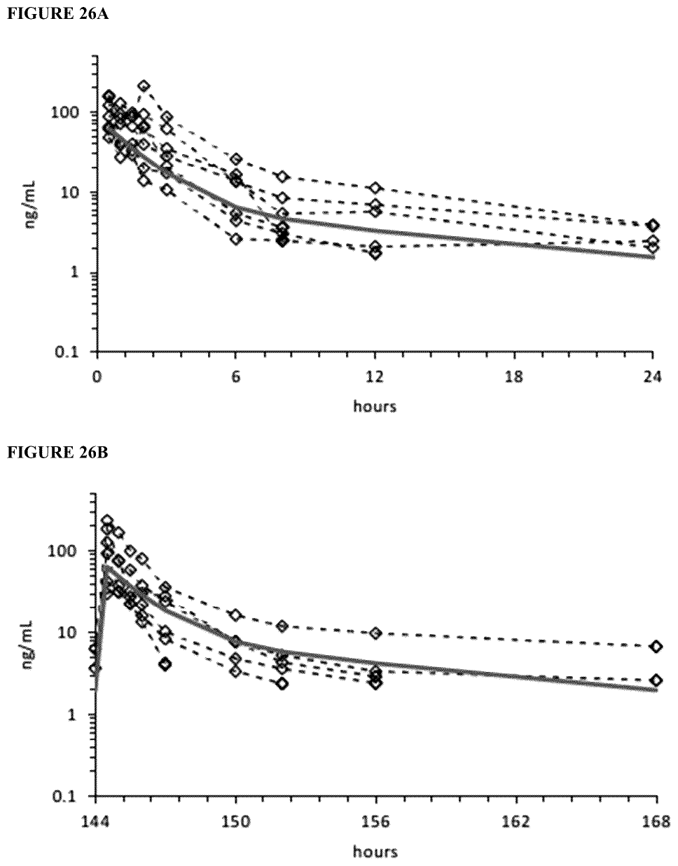

FIGS. 26A-26B show the mathematical model (heavy blue line) fit to the Cohort B data. The model was built based on geometric mean data, but timepoint data were not used in cases where plasma concentrations were below the limit of quantitation for four or more of the seven subjects.

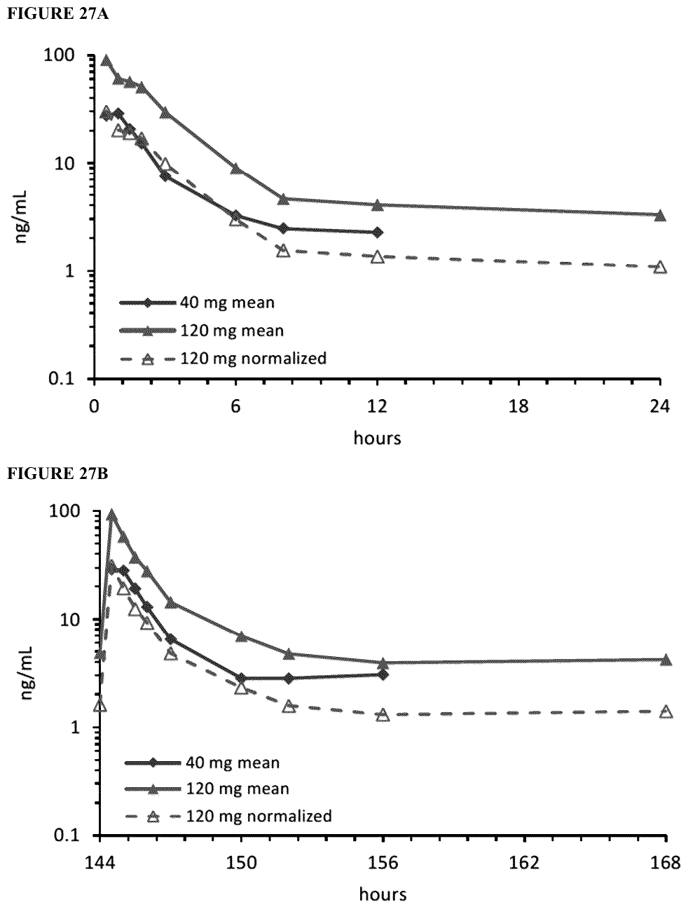

Good dose linearity was observed between the 40 mg and 120 mg doses, as illustrated in FIGS. 27A-27B. The PK obtained for the 120 mg dose, when normalized to a 40 mg dose (dotted blue lines), is nearly superimposable to the 40 mg PK at the earlier time points. The later time point data for the 40 mg dose is unreliable, since many of the subjects had plasma drug concentrations that were below the limit of quantitation.

PK parameters derived from the compartmental model are provided in Table 26. Note that for various mathematical reasons, the model estimates differ significantly from the mean results obtained from non-compartmental modeling.

TABLE 26
PK parameters for 120 mg QD dose derived from compartmental PK model

| Parameter | Day 1 | Day 2 | Day 3 | Day 4 | Day 5 | Day 6 | Day 7 | accumulation |
|---|---|---|---|---|---|---|---|---|
| AUC_{24 h} (h · ng/mL) | 210 | 227 | 230 | 231 | 231 | 231 | 231 | 110% |
| Cₘₐₓ (ng/mL) | 74.6 | 76.1 | 76.3 | 76.4 | 76.4 | 76.4 | 76.4 | 102% |
| Cₘᵢₙ (ng/mL) | N/A | 1.5 | 1.8 | 1.8 | 1.8 | 1.8 | 1.8 | N/A |
| tₘₐₓ (h) | 0.63 | 0.63 | 0.63 | 0.63 | 0.63 | 0.63 | 0.63 | N/A |

Predicted PK parameters for 360 mg daily input given as a single dose or as two doses are provided in Table 27. The model predicts significantly higher exposure for b.i.d. dosing.

TABLE 27
Predicted PK parameters for 360 mg daily dose of NTRX-07

| | 360 mg QD | | 180 mg b.i.d. | |
|---|---|---|---|---|
| Parameter | Day 1 | Day 7 | Day 1 | Day 7 |
| AUC_{24 h} (h · ng/mL) | 390 | 427 | 506 | 596 |
| Cₘₐₓ (ng/mL) | 133.8 | 137.0 | 100.0 | 103.0 |
| Cₘᵢₙ (ng/mL) | N/A | 3.0 | N/A | 8.0 |

### 6. Conclusion

Data from Cohort B (120 mg QD) show moderately high inter-subject variability and little or no accumulation over the course of seven days of dosing. Comparison to data obtained with a 40-mg daily dose suggests that the PK are linear across this dose range. Predictions for higher doses were provided based on output from a compartmental PK model. The compartmental PK model will be updated as additional data become available.

### Example 7: Interim PK Analysis NTRX-07 MAD Cohort C

As used in Example 7, an amount of “NTRX-07” refers to an amount of a spray-dried dispersion (SDD) of NTRX-07, containing 75% SDD and 25% NTRX-07, e.g., “40 mg NTRX-07” refers to 40 mg of an SDD containing 10 mg NTRX-07, “120 mg NTRX-07” refers to 120 mg of an SDD containing 30 mg NTRX-07, and “360 mg NTRX-07” refers to 360 mg of an SDD containing 90 mg NTRX-07.

### 1. Background

This report provides an interim review and analysis of pharmacokinetic (PK) data generated from subjects dosed with 360 mg NTRX-07 once daily for seven days as the third cohort of a multiple ascending dose (MAD) clinical trial.

### 2. Methodology

Raw bioanalytical data were received from Nuvisan on Dec. 19, 2022. Parameters derived from noncompartmental analysis of these data were calculated using standard equations. Specifically, area under the plasma concentration versus time curve (AUC) was calculated using the trapezoid rule. Maximal plasma concentration (Cmax) and time of maximal plasma concentration (Tmax) were taken directly from the data. Clearance divided by bioavailability (Cl/F) was calculated as the dose divided by the AUC. Apparent volume of distribution over bioavailability (Vd/F) was calculated as dose divided by Cmax.

Compartmental analysis was performed based on a two-compartment model with extravascular administration. Modeling was performed using a sophisticated MS Excel workbook developed by PharmaDirections. Geometric mean data from Cohorts A, B, and C were used to determine weighted least squares best fits for the PK parameters.

### 3. Summary of the PK Data

With oral administration of the dose every 24 hours for 7 days, blood samples were taken over a 24-hour interval on Days 1 and 7 and before and after dosing on Day 3. Specifically, sampling was performed as shown in Table 28.

TABLE 28
PK sampling scheme

| Study Day | Day 1 | Day 3 | Day 7 |
|---|---|---|---|
| Sampling Times (hr) | 0, 0.5, 1, 1.5, 2, | −0.5, 1.5 | 0, 0.5, 1, 1.5, 2, |
| | 3, 6, 8, 12, 24 | | 3, 6, 8, 12, 24 |

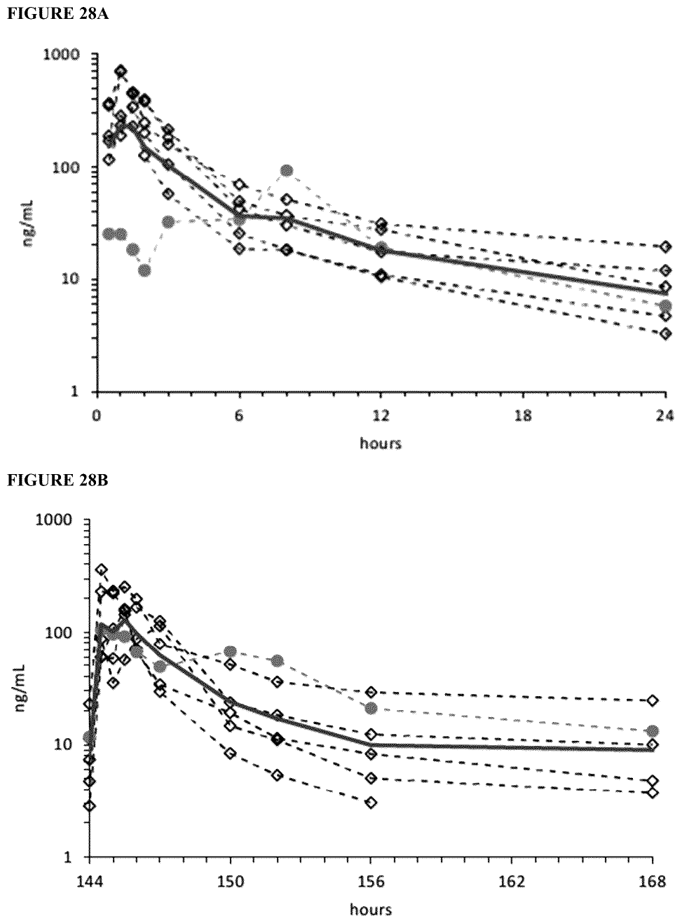

PK data for the seven subjects in the cohort on Day 1 and Day 7 are shown in FIGS. 28A-B. The red (solid) lines on the graphs represent the geometric means of the data. Inter-subject variability was moderately high. PK data for one subject (Subject 034) was notably aberrant on both Day 1 and Day 7. This subject's data are highlighted in green on the graphs but were not excluded from the analysis.

### 4. Non-Compartmental Analysis

Parameter values obtained from non-compartmental analysis of the PK data are shown in Tables 29-30.

TABLE 29
Day 1 PK parameters derived from non-compartmental analysis

| Parameter | sbj. 026 | sbj. 027 | sbj. 028 | sbj. 034 | sbj. 039 | sbj. 041 | mean ± S.D. |
|---|---|---|---|---|---|---|---|
| AUC_{24 h} (h · ng/mL) | 1629 | 857 | 925 | 657 | 1730 | 1949 | 1291 ± 541 |
| Cₘₐₓ (ng/mL) | 449 | 350 | 337 | 92.4 | 713 | 694 | 439 ± 236 |
| Tₘₐₓ (h) | 1.5 | 0.5 | 1.5 | 8 | 1 | 3 | 2.3 ± 2.8 |
| Cl/F (L/h) | 221.0 | 420.3 | 389.3 | 548.0 | 208.1 | 184.7 | 329 ± 146 |
| V_{d}/F (L) | 802 | 1029 | 1068 | 3896 | 505 | 519 | 1303 ± 1293 |

TABLE 30
Day 7 PK parameters derived from non-compartmental analysis

| Parameter | sbj. 026 | sbj. 027 | sbj. 028 | sbj. 034 | sbj. 039 | sbj. 041 | mean ± S.D. |
|---|---|---|---|---|---|---|---|
| AUC_{24 h} (h · ng/mL) | 783 | 604 | 354 | 516 | 876 | 1349 | 747 ± 349 |
| Cₘₐₓ (ng/mL) | 167 | 230 | 155 | 114 | 105 | 361 | 189 ± 95 |
| Tₘₐₓ (h) | 2 | 0.5 | 1.5 | 3 | 0.5 | 0.5 | 1.3 ± 1.0 |
| Cl/F (L/h) | 459.9 | 596.3 | 1017.7 | 697.8 | 410.9 | 266.9 | 575 ± 263 |
| V_{d}/F (L) | 2156 | 1565 | 2323 | 3158 | 3429 | 997 | 2271 ± 923 |

Note that large values for Cl/F and Vd/F can reflect poor bioavailability, broad distribution in the body or a combination of the two.

### 5. Predictive Compartmental Analysis

A two-compartment scheme upon which the mathematical compartmental model is based is shown in FIG. 21.

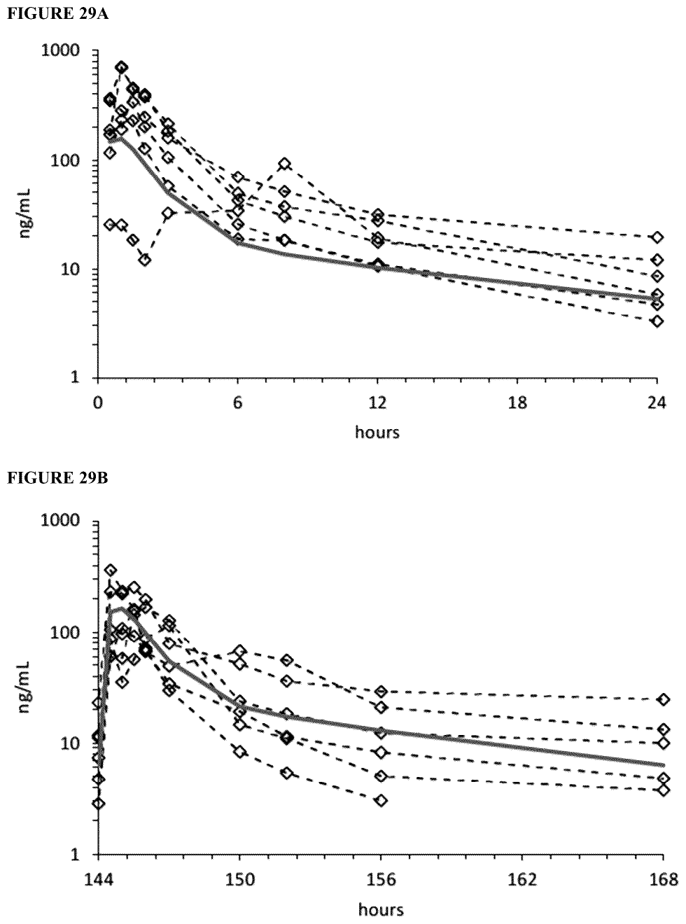

FIGS. 29A-B show the mathematical model (heavy blue line) fit to the Cohort C data. The model was built based on geometric mean data.

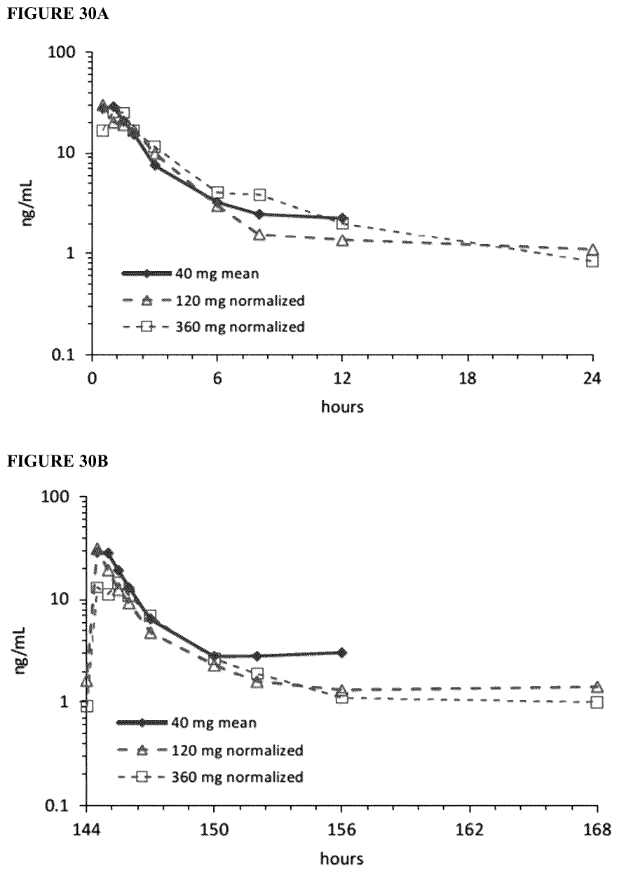

Dose linearity was observed among the 40, 120, and 360 mg doses, as illustrated in FIGS. 30A-B, wherein geometric mean PK results for the higher doses is normalized to a 40 mg dose.

PK parameters derived from the compartmental model are provided in Table 31. Note that for various mathematical reasons, the model estimates differ significantly from the mean results obtained from non-compartmental modeling.

TABLE 31
PK parameters for 360 mg QD dose derived from compartmental PK model

| Parameter | Day 1 | Day 2 | Day 3 | Day 4 | Day 5 | Day 6 | Day 7 | accumulation |
|---|---|---|---|---|---|---|---|---|
| AUC_{24 h} (h · ng/mL) | 559 | 616 | 628 | 631 | 631 | 631 | 631 | 113% |
| Cₘₐₓ (ng/mL) | 162.0 | 166.4 | 167.4 | 167.6 | 167.6 | 167.6 | 167.6 | 103% |
| Cₘᵢₙ (ng/mL) | N/A | 4.7 | 5.4 | 5.7 | 5.7 | 5.7 | 5.7 | N/A |
| tₘₐₓ (h) | 0.79 | 0.79 | 0.79 | 0.79 | 0.79 | 0.79 | 0.79 | N/A |

Updated predicted PK parameters for 360-mg daily input given as a single dose or as two doses are provided in Table 32. The model predicts somewhat higher exposure for b.i.d. dosing.

TABLE 32
Predicted PK parameters for 360 mg daily dose of NTRX-07

| | 360 mg QD | | 180 mg b.i.d. | |
|---|---|---|---|---|
| Parameter | Day 1 | Day 7 | Day 1 | Day 7 |
| AUC_{24 h} (h · ng/mL) | 559 | 631 | 592 | 724 |
| Cₘₐₓ (ng/mL) | 162.0 | 167.6 | 100.1 | 104.8 |
| Cₘᵢₙ (ng/mL) | N/A | 5.7 | N/A | 10.8 |

### 6. Conclusion

Data from Cohort C (360 mg QD) show moderately high inter-subject variability and little or no accumulation over the course of seven days of dosing. Comparison to data obtained with a 40-mg and 120-mg daily dose suggests that the PK are linear across this dose range.

### Example 8: Interim PK Analysis NTRX-07 MAD Cohort D

### 1. Background

This report provides an interim review and analysis of pharmacokinetic (PK) data generated from subjects dosed with 360 mg NTRX-07 spray-dried dispersion (SDD), containing 90 mg of NTRX-07, once daily for seven days as the fourth cohort of a multiple ascending dose (MAD) clinical trial.

### 2. Methodology

Raw bioanalytical data were received from Nuvisan on Jun. 30, 2023. Parameters derived from noncompartmental analysis of these data were calculated using standard equations. Specifically, area under the plasma concentration versus time curve (AUC) was calculated using the trapezoid rule. Maximal plasma concentration (Cmax) and time of maximal plasma concentration (Tmax) were taken directly from the data. Clearance divided by bioavailability (Cl/F) was calculated as the dose divided by the AUC. Apparent volume of distribution over bioavailability (Vd/F) was calculated as dose divided by Cmax.

Compartmental analysis was performed based on a two-compartment model with extravascular administration. Modeling was performed using a sophisticated MS Excel workbook developed by PharmaDirections. Geometric mean data from Cohorts A, B, C, and D were used to determine weighted least squares best fits for the PK parameters.

### 3. Summary of the PK Data

With oral administration of the dose every 24 hours for 7 days, blood samples were taken over a 24-hour interval on Days 1 and 7 and before and after dosing on Day 3. Specifically, sampling was performed as shown in Table 33.

TABLE 33
PK sampling scheme

| Study Day | Day 1 | Day 3 | Day 7 |
|---|---|---|---|
| Sampling Times (hr) | 0, 0.5, 1, 1.5, 2, | −0.5. 1.5 | 0, 0.5, 1, 1.5, 2, |
| | 3, 6, 8, 12, 24 | | 3, 6, 8, 12, 24 |

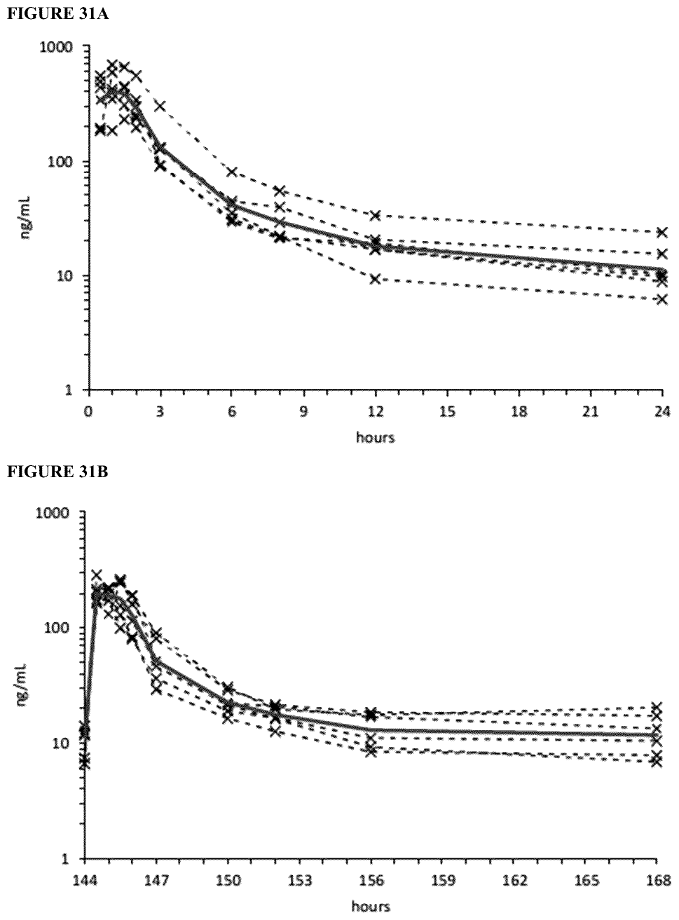

PK data for the six subjects in the cohort on Day 1 and Day 7 are shown in FIGS. 31A-B. The (solid) red lines on the graphs represent the geometric means of the data. Inter-subject variability was moderate.

### 4. Non-Compartmental Analysis

Parameter values obtained from non-compartmental analysis of the PK data are shown in Tables 34-35.

TABLE 34
Day 1 PK parameters derived from non-compartmental analysis

| Parameter | sbj. 046 | sbj. 048 | sbj. 051 | sbj. 052 | sbj. 056 | sbj. 058 | mean ± S.D. |
|---|---|---|---|---|---|---|---|
| AUC_{24 h} (h · ng/mL) | 1004 | 1255 | 2727 | 1349 | 1580 | 1422 | 1556 ± 329 |
| Cₘₐₓ (ng/mL) | 234 | 491 | 685 | 435 | 434 | 587 | 478 ± 154 |
| Tₘₐₓ (h) | 2 | 0.5 | 1 | 1.5 | 0.5 | 1 | 1.1 ± 0.6 |
| Cl/F (L/h) | 358.6 | 286.8 | 132.0 | 266.9 | 227.8 | 253.3 | 254 ± 74 |
| V_{d}/F (L) | 1538 | 733 | 526 | 828 | 829 | 613 | 845 ± 360 |

TABLE 35
Day 7 PK parameters derived from non-compartmental analysis

| Parameter | sbj. 046 | sbj. 048 | sbj. 051 | sbj. 052 | sbj. 056 | sbj. 058 | mean ± S.D. |
|---|---|---|---|---|---|---|---|
| AUC_{24 h} (h · ng/mL) | 1008 | 1854 | 1659 | 1603 | 1328 | 1130 | 1430 ± 329 |
| Cₘₐₓ (ng/mL) | 286 | 250 | 262 | 246 | 176 | 221 | 240 ± 38 |
| Tₘₐₓ (h) | 0.5 | 1.5 | 1.5 | 1.5 | 0.5 | 1 | 1.1 ± 0.5 |
| Cl/F (L/h) | 357.3 | 194.2 | 217.1 | 224.5 | 271.1 | 318.6 | 245 ± 50 |
| V_{d}/F (L) | 1259 | 1440 | 1374 | 1463 | 2045 | 1629 | 1535 ± 278 |

Note that large values for Cl/F and Vd/F can reflect poor bioavailability, broad distribution in the body or a combination of the two.

### 5. Predictive Compartmental Analysis

A two-compartment scheme upon which the mathematical compartmental model is based is shown in FIG. 21.

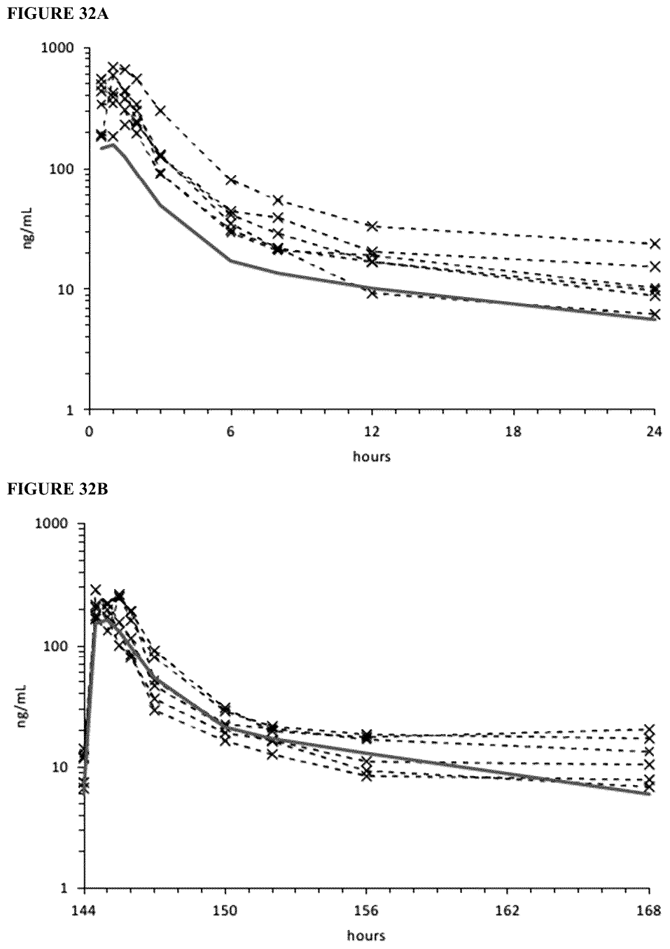

FIGS. 32A-B show the mathematical model (heavy blue line) fit to the Cohort D data. The model was built based on geometric mean data.

The data diverge from the model in two respects:
The model underpredicts Day 1 exposures.
The model does not capture the near-zero Day 7 terminal elimination phase slope.

The apparent decrease in exposure from Day 1 to Day 7 for this cohort hints at a possibility of enzyme induction. The model predicts slight accumulation with repeat dosing, but it appears that exposure is decreasing rather than increasing over time.

### 6. Conclusion

Data from Cohort D (360 mg QD of NTRX-07 SDD) show moderate inter-subject variability and no accumulation over the course of seven days of dosing. Intersubject variability appears to be lower for this cohort than for prior cohorts.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims. Those skilled in the art will also recognize that all combinations of embodiments described herein are within the scope of the invention.

1. A method of treating neurodegeneration in a human subject in need thereof, the method comprising:
a) on day 1, administering to the subject a first daily dose that is a pharmaceutical composition comprising 80-150 mg of the compound of Formula I 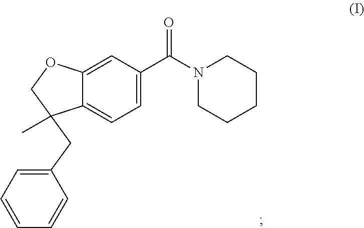 [SMILES: CC1(Cc2ccccc2)COc2cc(C(=O)N3CCCCC3)ccc21]
b) starting on day 2, administering to the subject a subsequent daily dose that is a pharmaceutical composition comprising 80-150 mg of the compound of Formula I;
c) 6-28 days after the first daily dose, increasing the subsequent daily dose to a pharmaceutical composition comprising 125-225 mg of the compound of Formula I.

2-36. (canceled)

37. The method of claim 1, wherein the neurodegeneration comprises Alzheimer's Disease, cognitive impairment, or a combination thereof.

38-39. (canceled)

40. The method of claim 37, wherein the neurodegeneration comprises Alzheimer's Disease with cognitive impairment.

41. The method of claim 1, the method comprising:
a) on day 1, administering to the subject a first daily dose that is a pharmaceutical composition comprising 90-135 mg of the compound of Formula I 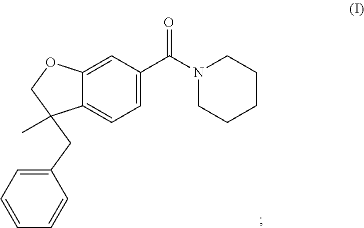 [SMILES: CC1(Cc2ccccc2)COc2cc(C(=O)N3CCCCC3)ccc21]
b) starting on day 2, administering to the subject a subsequent daily dose that is a pharmaceutical composition comprising 90-135 mg of the compound of Formula I;
c) 6-28 days after the first daily dose, increasing the subsequent daily dose to a pharmaceutical composition comprising 135-202.5 mg of the compound of Formula I.

42-68. (canceled)

69. The method of claim 41, wherein the neurodegeneration comprises Alzheimer's Disease, cognitive impairment, or a combination thereof.

70-72. (canceled)

73. A method of treating neurodegeneration in a human subject in need thereof, the method comprising:
a) on day 1, administering to the subject a first daily dose that is a pharmaceutical composition comprising 80-150 mg of the compound of Formula I 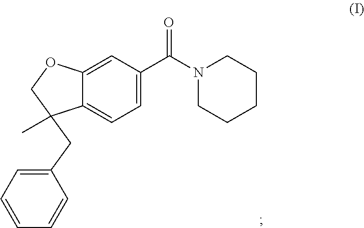 [SMILES: CC1(Cc2ccccc2)COc2cc(C(=O)N3CCCCC3)ccc21]
b) starting on day 2, administering to the subject a subsequent daily dose that is a pharmaceutical composition comprising 80-150 mg of the compound of Formula I;
c) 6-28 days after the first daily dose, measuring the serum level of the compound of Formula I in the subject at a time point in the period 1-12 hours after that day's daily dose;
d) plotting the serum level of the compound of Formula I measured in c) against the graph of serum levels in FIG. 1 at the time point corresponding to the time point in c);
e) if the serum level is below the geometric mean in FIG. 1, increasing the subsequent daily dosage by an amount corresponding to the percentage increase required to reach the geometric mean.

74-97. (canceled)

98. The method of claim 73, wherein the neurodegeneration comprises Alzheimer's Disease, cognitive impairment, or a combination thereof.

99-100. (canceled)

101. The method of claim 98, wherein the neurodegeneration comprises Alzheimer's Disease with cognitive impairment.

102. A method of treating neurodegeneration in a human subject in need thereof, the method comprising:
a) on day 1, administering to the subject a first daily dose that is a pharmaceutical composition comprising 90-180 mg of the compound of Formula I 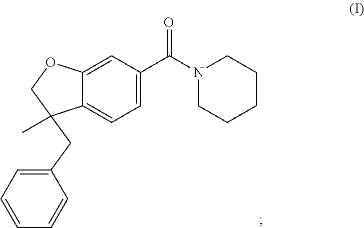 [SMILES: CC1(Cc2ccccc2)COc2cc(C(=O)N3CCCCC3)ccc21]
b) starting on day 2, administering to the subject a subsequent daily dose that is a pharmaceutical composition comprising 90-180 mg of the compound of Formula I;
c) 6-28 days after the first daily dose, increasing the subsequent daily dose to a pharmaceutical composition comprising 135-270 mg of the compound of Formula I (e.g., increasing the subsequent daily dose to a pharmaceutical composition comprising 150-200% of the amount of the compound of Formula I in the subsequent daily dose administered in step b).

103-144. (canceled)

145. A method of treating neurodegeneration in a human subject in need thereof, the method comprising:
a) on day 1, administering to the subject a first daily dose that is a pharmaceutical composition comprising 90-180 mg of the compound of Formula I 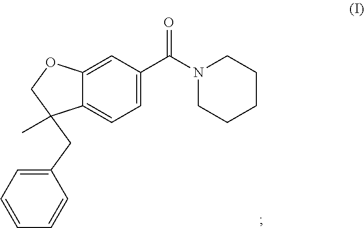 [SMILES: CC1(Cc2ccccc2)COc2cc(C(=O)N3CCCCC3)ccc21]
b) starting on day 2, administering to the subject a subsequent daily dose that is a pharmaceutical composition comprising 90-180 mg of the compound of Formula I;
c) 6-28 days after the first daily dose, measuring the AUC of the compound of Formula I in the subject; and
d) if the AUC is less than 1200 h·ng/mL, increasing the subsequent daily dose by an amount corresponding to the percentage increase required to reach an AUC of at least 1200 h·ng/mL.

146-174. (canceled)

175. A method of treating neurodegeneration in a human subject in need thereof, the method comprising:
a) on day 1, administering to the subject a first daily dose that is a pharmaceutical composition comprising 90-180 mg of the compound of Formula I 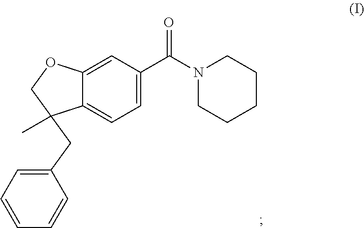 [SMILES: CC1(Cc2ccccc2)COc2cc(C(=O)N3CCCCC3)ccc21]
b) starting on day 2, administering to the subject a subsequent daily dose that is a pharmaceutical composition comprising 90-180 mg of the compound of Formula I;
c) 6-28 days after the first daily dose, increasing the subsequent daily dose to a pharmaceutical composition comprising 125-225% of the amount of the compound of Formula I in the subsequent daily dose administered in step b.

176-217. (canceled)

218. The method of claim 102, wherein the neurodegeneration comprises Alzheimer's Disease, cognitive impairment, or a combination thereof.

219-220. (canceled)

221. The method of claim 218, wherein the neurodegeneration comprises Alzheimer's Disease with cognitive impairment.

222. The method of claim 145, wherein the neurodegeneration comprises Alzheimer's Disease, cognitive impairment, or a combination thereof.

223. The method of claim 222, wherein the neurodegeneration comprises Alzheimer's Disease with cognitive impairment.

224. The method of claim 175, wherein the neurodegeneration comprises Alzheimer's Disease, cognitive impairment, or a combination thereof.

225. The method of claim 224, wherein the neurodegeneration comprises Alzheimer's Disease with cognitive impairment.
